SEQUENCE LISTING (104 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 15 aa, Homo sapiens
EPKSCDKTHTCPPCP

SEQ ID NO: 6 - protein, 10 aa, Homo sapiens
DKTHTCPPCP

SEQ ID NO: 7 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 8 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 9 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 11 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 13 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 15 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 17 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 21 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 23 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPGK

SEQ ID NO: 24 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWANYKTTPPVLDSDGSFFLYSKLTVTKEEWQQGFVFSCSVMHEALHNHYTQKSLSLSPG

SEQ ID NO: 25 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 26 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 27 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 28 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 29 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 30 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 31 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 32 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 33 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 34 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 35 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 36 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 37 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 38 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 39 - protein, 653 aa, synthetic construct
  VARIANT 33: note = H or Q
  VARIANT 622: note = A or T
MRPLRPRAALLALLASLLAAPPVAPAEAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPVPRGPPSPGNP

SEQ ID NO: 40 - protein, 627 aa, synthetic construct
  VARIANT 7: note = H or Q
  VARIANT 596: note = A or T
EAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPVPRGPPSPGNP

SEQ ID NO: 41 - protein, 627 aa, Homo sapiens
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNP

SEQ ID NO: 42 - protein, 627 aa, Homo sapiens
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 43 - protein, 627 aa, Homo sapiens
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNP

SEQ ID NO: 44 - protein, 627 aa, Homo sapiens
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 45 - protein, 617 aa, synthetic construct
  VARIANT 7: note = H or Q
  VARIANT 596: note = A or T
EAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPV

SEQ ID NO: 46 - protein, 617 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPV

SEQ ID NO: 47 - protein, 617 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPV

SEQ ID NO: 48 - protein, 617 aa, synthetic construct
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPV

SEQ ID NO: 49 - protein, 617 aa, synthetic construct
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPV

SEQ ID NO: 50 - protein, 859 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 858 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 52 - protein, 859 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 858 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 54 - protein, 849 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 55 - protein, 848 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 56 - protein, 849 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 848 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 58 - protein, 859 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 858 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 60 - protein, 859 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 858 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 62 - protein, 849 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 848 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 64 - protein, 849 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 65 - protein, 848 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 66 - protein, 859 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSEAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 67 - protein, 859 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSEAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNP

SEQ ID NO: 68 - protein, 859 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSEAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 69 - protein, 859 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSEAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNP

SEQ ID NO: 70 - protein, 19 aa, Mus sp.
MGWSCIILFLVATATGAYA

SEQ ID NO: 71 - skipped

SEQ ID NO: 72 - protein, 5 aa, synthetic construct
GGGGS

SEQ ID NO: 73 - protein, 10 aa, synthetic construct
GGGGSGGGGS

SEQ ID NO: 74 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 620 aa, synthetic construct
  VARIANT 7: note = H or Q
  VARIANT 596: note = A or T
EAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPVPRG

SEQ ID NO: 79 - protein, 620 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRG

SEQ ID NO: 80 - protein, 620 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRG

SEQ ID NO: 81 - protein, 620 aa, synthetic construct
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRG

SEQ ID NO: 82 - protein, 620 aa, synthetic construct
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRG

SEQ ID NO: 83 - protein, 852 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 84 - protein, 851 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 85 - protein, 852 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 851 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 87 - protein, 852 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 851 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 89 - protein, 852 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 90 - protein, 851 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 91 - protein, 859 aa, synthetic construct
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 858 aa, synthetic construct
EAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 93 - protein, 635 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGSHHHHHH

SEQ ID NO: 94 - protein, 635 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGSHHHHHH

SEQ ID NO: 95 - protein, 635 aa, synthetic construct
  VARIANT 1: note = E or absent
XAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNPGSHHHHHH

SEQ ID NO: 96 - protein, 6 aa, synthetic construct
HHHHHH

SEQ ID NO: 97 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPGK

SEQ ID NO: 98 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESYGTEWANYKTTPPVLDSDGSFFLVSKLTVTKEEWQQGFVFSCSVMHEALHNHYTQKSLSLSPG

SEQ ID NO: 99 - protein, 627 aa, synthetic construct
  VARIANT 1: note = H or Q
  VARIANT 7: note = A or T
  VARIANT 596: note = E or absent
XAPHLVXVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRXLDYWARPGPFSDPVPYLEVPVPRGPPSPGNP

SEQ ID NO: 100 - protein, 858 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 859 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGK

SEQ ID NO: 102 - protein, 858 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPG

SEQ ID NO: 103 - protein, 859 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWANYKTTPPVLDSDGSFFLYSKLTVTKEEWQQGFVFSCSVMHEALHNHYTQKSLSLSPGKGGGGSEAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNP

SEQ ID NO: 104 - protein, 859 aa, synthetic construct
EAPHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRTLDYWARPGPFSDPVPYLEVPVPRGPPSPGNPGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0136963 A1
(43) Pub. Date: May 1, 2025

(54) FUSION PROTEINS COMPRISING ALPHA-L-IDURONIDASE ENZYMES AND METHODS

(71) Applicant: DENALI THERAPEUTICS INC., South San Francisco, CA (US)

(72) Inventors: Gowrisudha ADUSUMILLI, Hayward, CA (US); Oliver Brayer DAVIS, Oakland, CA (US); Mihalis S. KARIOLIS, San Mateo, CA (US); Cathal S. MAHON, San Francisco, CA (US); Shrishti TYAGI, Santa Clara, CA (US); Kensuke YAMANOKUCHI, South San Francisco, CA (US)

(73) Assignee: DENALI THERAPEUTICS INC., South San Francisco, CA (US)

(21) Appl. No.: 18/719,958

(22) PCT Filed: Dec. 16, 2022
(86) PCT No.: PCT/US2022/053196

§ 371 Date: Jun. 14, 2024

Related U.S. Application Data
(60) Provisional application No. 63/291,283, filed on Dec. 17, 2021.

(51) Int. Cl. C12N 9/24; A61K 38/00; A61P 3/00; C07K 16/28

(52) U.S. Cl. CPC C12N 9/2402; A61P 3/00; C07K 16/2881; A61K 38/00; C07K 2317/52; C07K 2319/30; C12Y 302/01076

(57) ABSTRACT

Provided herein are proteins, which are capable of being transported across the blood-brain barrier (BBB) and comprise an alpha-L-iduronidase (IDUA) enzyme-Fc fusion polypeptide. Certain embodiments also provide methods of using such proteins to treat MPS I.

### CROSS-REFERENCE TO RELATED APPLICATION

This application claims priority to U.S. Provisional Application Ser. No. 63/291,283, filed Dec. 17, 2021. The entire content of the application referenced above is hereby incorporated by reference herein.

### SEQUENCE LISTING

The instant application contains a Sequence Listing which has been submitted electronically in XML format and is hereby incorporated by reference in its entirety. Said XML copy, created on Jan. 8, 2025, is named 02900_042US1_SL.xml and is 145,864 bytes in size.

### BACKGROUND

Mucopolysaccharidosis I (MPS I) (or Hurler syndrome) is a lysosomal storage disorder caused by genetic mutations in the IDUA gene. These mutations reduce or eliminate alpha-L-iduronidase (IDUA) protein function, which results in the accumulation of the glycosaminoglycans dermatan sulfate and heparan sulfate and to alterations in multiple organs and tissues, including the skeleton, heart, respiratory system, and brain. Treatments for MPS I remain largely supportive; while the deficient enzyme may be administered intravenously, it has little effect on the brain due to difficulties in delivering the recombinant enzyme across the blood-brain barrier (BBB). Accordingly, there is a need for more effective therapies that treat MPS I symptoms and IDUA deficiencies in both the CNS and the periphery.

### SUMMARY

Thus, provided herein is a specific enzyme replacement therapy, which has the capability of crossing the BBB and treating both the peripheral and CNS manifestations of MPS I. In particular, certain embodiments provide a protein comprising (a) a first Fc polypeptide linked to an alpha-L-iduronidase (IDUA) amino acid sequence, an IDUA variant amino acid sequence, or a catalytically active fragment thereof, and (b) a second Fc polypeptide; wherein the first and/or second Fc polypeptide is a modified Fc that is capable of binding (e.g., specifically binding) to a blood-brain barrier (BBB) receptor, e.g., a transferrin receptor (TfR). In certain embodiments, the second Fc polypeptide forms an Fc dimer with the first Fc polypeptide.

In certain embodiments, the first Fc polypeptide is a modified Fc that is capable of binding (e.g., specifically binding) to TfR. In certain embodiments, the first Fc polypeptide is a modified Fc comprising a sequence having at least 90% identity to SEQ ID NO: 28 or 98 (e.g., SEQ ID NO:28) and is capable of specifically binding to a TfR.

In certain embodiments, the first Fc polypeptide 1) comprises a sequence having at least 90% identity to SEQ ID NO: 28 or 98 (e.g., SEQ ID NO:28); 2) is capable of specifically binding to a TfR; and 3) has Ala at position 389, according to EU numbering. In certain embodiments, the first Fc polypeptide further comprises Glu at position 380; and Asn at position 390, according to EU numbering. In certain embodiments, the first Fc polypeptide further comprises at the following positions, according to EU numbering: Tyr at position 384; Thr at position 386; Glu at position 387; Trp at position 388; Thr at position 413; Glu at position 415; Glu at position 416; and Phe at position 421.

In certain embodiments, the second Fc polypeptide is a modified Fc that is capable of binding (e.g., specifically binding) to TfR. In certain embodiments, the second Fc polypeptide is a modified Fc comprising a sequence having at least 90% identity to SEQ ID NO: 28 or 98 (e.g., SEQ ID NO:28) and is capable of specifically binding to a TfR.

In certain embodiments, the second Fc polypeptide 1) comprises a sequence having at least 90% identity to SEQ ID NO: 28 or 98 (e.g., SEQ ID NO:28); 2) is capable of specifically binding to a TfR; and 3) has Ala at position 389, according to EU numbering. In certain embodiments, the second Fc polypeptide further comprises Glu at position 380; and Asn at position 390, according to EU numbering. In certain embodiments, the second Fc polypeptide further comprises at the following positions, according to EU numbering: Tyr at position 384; Thr at position 386; Glu at position 387; Trp at position 388; Thr at position 413; Glu at position 415; Glu at position 416; and Phe at position 421.

Certain embodiments provide a protein comprising:
a. a first Fc polypeptide linked to an alpha-L-iduronidase (IDUA) amino acid sequence, an IDUA variant amino acid sequence, or a catalytically active fragment thereof; and
b. a second Fc polypeptide that comprises a sequence having at least 90% identity to SEQ ID NO: 28 and that is capable of specifically binding to a transferrin receptor (TfR).

In certain embodiments, the second Fc polypeptide has Ala at position 389, according to EU numbering.

In certain embodiments, the second Fc polypeptide further comprises Glu at position 380; and Asn at position 390, according to EU numbering.

In certain embodiments, the second Fc polypeptide further comprises at the following positions, according to EU numbering: Tyr at position 384; Thr at position 386; Glu at position 387; Trp at position 388; Thr at position 413; Glu at position 415; Glu at position 416; and Phe at position 421.

In certain embodiments, the protein is capable of being transported across the blood-brain barrier of a subject.

In certain embodiments, the protein binds to a TfR with an affinity of from about 100 nM to about 500 nM.

In certain embodiments, the protein binds to a TfR with an affinity of from about 200 nM to about 400 nM.

In certain embodiments, the second Fc polypeptide binds to the apical domain of the TfR.

In certain embodiments, the binding of the protein to the TfR does not substantially inhibit binding of transferrin to the TfR.

In certain embodiments, the IDUA amino acid sequence comprises an amino acid sequence having at least 80%, 85%, 90%, or 95% identity to any one of SEQ ID NOS: 39, 40, 45, 78, and 99.

In certain embodiments, the IDUA amino acid sequence comprises the amino acid sequence of any one of SEQ ID NOS:39, 40, 45, 78, and 99.

In certain embodiments, the IDUA amino acid sequence comprises an amino acid sequence having at least 80%, 85%, 90%, or 95% identity to any one of SEQ ID NOS: 41-44.

In certain embodiments, the IDUA amino acid sequence comprises the amino acid sequence of any one of SEQ ID NOS: 41-44.

In certain embodiments, the IDUA amino acid sequence comprises an amino acid sequence having at least 80%, 85%, 90%, or 95% identity to any one of SEQ ID NOS: 46-49.

In certain embodiments, the IDUA amino acid sequence comprises the amino acid sequence of any one of SEQ ID NOS: 46-49.

In certain embodiments, the IDUA amino acid sequence comprises an amino acid sequence having at least 80%, 85%, 90%, or 95% identity to any one of SEQ ID NOS: 79-82.

In certain embodiments, the IDUA amino acid sequence comprises the amino acid sequence of any one of SEQ ID NOS: 79-82.

In certain embodiments, the first Fc polypeptide is linked to the IDUA amino acid sequence, IDUA variant amino acid sequence, or a catalytically active fragment thereof by a peptide bond or by a polypeptide linker. In certain embodiments, the first Fc polypeptide is linked to the IDUA amino acid sequence, IDUA variant amino acid sequence, or a catalytically active fragment thereof by a peptide bond. In certain embodiments, the first Fc polypeptide is linked to the IDUA amino acid sequence, IDUA variant amino acid sequence, or a catalytically active fragment thereof by a polypeptide linker.

In certain embodiments, the polypeptide linker is a flexible polypeptide linker.

In certain embodiments, the flexible polypeptide linker is a glycine-rich linker.

In certain embodiments, the polypeptide linker is GS, G₄S (SEQ ID NO:72) or (G₄S)₂ (SEQ ID NO:73).

In certain embodiments, the N-terminus of the first Fc polypeptide is linked to the IDUA amino acid sequence, IDUA variant amino acid sequence, or a catalytically active fragment thereof.

In certain embodiments, the C-terminus of the first Fc polypeptide is linked to the IDUA amino acid sequence, IDUA variant amino acid sequence, or a catalytically active fragment thereof.

In certain embodiments, a fusion protein as described herein comprises a single IDUA amino acid sequence, IDUA variant amino acid sequence, or a catalytically active fragment thereof.

In certain embodiments, the second Fc polypeptide forms an Fc dimer with the first Fc polypeptide.

In certain embodiments, the first Fc polypeptide and the second Fc polypeptide each contain modifications that promote heterodimerization.

In certain embodiments, one of the Fc polypeptides has a T366W substitution and the other Fc polypeptide has T366S, L368A, and Y407V substitutions, according to EU numbering.

In certain embodiments, the first Fc polypeptide contains the T366S, L368A, and Y407V substitutions and the second Fc polypeptide contains the T366W substitution.

In certain embodiments, the first Fc polypeptide comprises an amino acid sequence having at least 95% or 100% identity to any one of SEQ ID NOS: 9-16 and 19-22; and the second Fc polypeptide comprises an amino acid sequence having at least 95% or 100% identity to any one of SEQ ID NOS: 25-32 and 35-38.

In certain embodiments, the first Fc polypeptide contains the T366W substitution and the second Fc polypeptide contains the T366S, L368A, and Y407V substitutions.

In certain embodiments, the first Fc polypeptide comprises an amino acid sequence having at least 95% or 100% identity to any one of SEQ ID NOS: 17-18 and 74-75; and the second Fc polypeptide comprises an amino acid sequence having at least 95% or 100% identity to any one of SEQ ID NOS: 33-34 and 97-98.

In certain embodiments, the first Fc polypeptide and/or the second Fc polypeptide comprises a native FcRn binding site.

In certain embodiments, the first Fc polypeptide and the second Fc polypeptide do not have effector function.

In certain embodiments, the first Fc polypeptide and/or the second Fc polypeptide includes a modification that reduces effector function.

In certain embodiments, the modification that reduces effector function is the substitutions of Ala at position 234 and Ala at position 235; Ala at position 234, Ala at position 235 and Gly at position 329; or Ala at position 234, Ala at position 235 and Ser at position 329, according to EU numbering.

In certain embodiments, the first Fc polypeptide comprises an amino acid sequence having at least 95% or 100% identity to any one of SEQ ID NOS: 11-16, and 19-22.

In certain embodiments, the first Fc polypeptide comprises an amino acid sequence having at least 95% or 100% identity to any one of SEQ ID NOS: 11, 12, 19, and 20.

In certain embodiments, the first Fc polypeptide comprises an amino acid sequence having at least 95% or 100% identity to any one of SEQ ID NOS:15, 16, 21, and 22.

In certain embodiments, the first Fc polypeptide linked to the IDUA amino acid sequence comprises an amino acid sequence having at least 95% or 100% identity to any one of SEQ ID NOS: 50-69 and 83-92.

In certain embodiments, the first Fc polypeptide linked to the IDUA amino acid sequence comprises an amino acid sequence having at least 95% or 100% identity to any one of SEQ ID NOS: 50-53.

In certain embodiments, the first Fc polypeptide linked to the IDUA amino acid sequence comprises an amino acid sequence having at least 95% or 100% identity to any one of SEQ ID NOS: 54-57.

In certain embodiments, the first Fc polypeptide linked to the IDUA amino acid sequence comprises an amino acid sequence having at least 95% or 100% identity to any one of SEQ ID NOS: 83-86.

In certain embodiments, the first Fc polypeptide linked to the IDUA amino acid sequence comprises an amino acid sequence having at least 95% or 100% identity to any one of SEQ ID NOS: 58-61 and 91-92.

In certain embodiments, the first Fc polypeptide linked to the IDUA amino acid sequence comprises an amino acid sequence having at least 95% or 100% identity to any one of SEQ ID NOS: 62-65.

In certain embodiments, the first Fc polypeptide linked to the IDUA amino acid sequence comprises an amino acid sequence having at least 95% or 100% identity to any one of SEQ ID NOS: 87-90.

In certain embodiments, the first Fc polypeptide linked to the IDUA amino acid sequence comprises an amino acid sequence having at least 95% or 100% identity to any one of SEQ ID NOS: 66-67.

In certain embodiments, the first Fc polypeptide linked to the IDUA amino acid sequence comprises an amino acid sequence having at least 95% or 100% identity to any one of SEQ ID NOS: 68-69.

In certain embodiments, the second Fc polypeptide comprises an amino acid sequence having at least 95% or 100% identity to any one of SEQ ID NOS: 27-32 and 35-38.

In certain embodiments, the second Fc polypeptide comprises an amino acid sequence having at least 95% or 100% identity to any one of SEQ ID NOS: 27, 28, 35 and 36.

In certain embodiments, the second Fc polypeptide comprises an amino acid sequence having at least 95% or 100% identity to any one of SEQ ID NOS:31, 32, 37 and 38.

In certain embodiments, the first Fc polypeptide linked to the IDUA amino acid sequence comprises the amino acid sequence of any one of SEQ ID NOS: 50-65 and 83-92; and the second Fc polypeptide comprises the amino acid sequence of any one of SEQ ID NOS: 35-38.

In certain embodiments, the first Fc polypeptide linked to the IDUA amino acid sequence comprises the amino acid sequence of any one of SEQ ID NOS: 50-57 and 83-86; and the second Fc polypeptide comprises the amino acid sequence of any one of SEQ ID NOS: 35-36.

In certain embodiments, the first Fc polypeptide linked to the IDUA amino acid sequence comprises the amino acid sequence of any one of SEQ ID NOS: 58-65 and 87-92; and the second Fc polypeptide comprises the amino acid sequence of any one of SEQ ID NOS: 37-38.

In certain embodiments, the first Fc polypeptide linked to the IDUA amino acid sequence comprises the amino acid sequence of any one of SEQ ID NOS: 66-69; and the second Fc polypeptide comprises the amino acid sequence of any one of SEQ ID NOS: 35-38.

In certain embodiments, the first Fc polypeptide linked to the IDUA amino acid sequence comprises the amino acid sequence of any one of SEQ ID NOS: 66-67; and the second Fc polypeptide comprises the amino acid sequence of any one of SEQ ID NOS: 35-36.

In certain embodiments, the first Fc polypeptide linked to the IDUA amino acid sequence comprises the amino acid sequence of any one of SEQ ID NOS: 68-69; and the second Fc polypeptide comprises the amino acid sequence of any one of SEQ ID NOS: 37-38.

In certain embodiments, uptake of the IDUA amino acid sequence into the brain is at least five-fold greater as compared to the uptake of the IDUA amino acid sequence in the absence of the first Fc polypeptide and the second Fc polypeptide or as compared to the uptake of the IDUA enzyme without the modifications to the second Fc polypeptide that result in TfR binding.

In certain embodiments, the first Fc polypeptide is not modified to bind to a blood-brain barrier (BBB) receptor and the second Fc polypeptide is modified to specifically bind to a TfR.

In certain embodiments, the protein does not include an immunoglobulin heavy and/or light chain variable region sequence or an antigen-binding portion thereof.

Certain embodiments also provide a polypeptide comprising an Fc polypeptide that is linked to an alpha-L-iduronidase (IDUA) amino acid sequence, an IDUA variant amino acid sequence, or a catalytically active fragment thereof, wherein the Fc polypeptide comprises a sequence having at least 90% identity to SEQ ID NO: 12 and contains one or more modifications that promote its heterodimerization to another Fc polypeptide.

In certain embodiments, the Fc polypeptide is linked to the IDUA enzyme, the IDUA variant amino acid sequence, or the catalytically active fragment thereof by a peptide bond or by a polypeptide linker.

In certain embodiments, the polypeptide comprises from N- to C-terminus: the IDUA enzyme, the IDUA variant amino acid sequence, or the catalytically active fragment thereof; a polypeptide linker; and the Fc polypeptide.

In certain embodiments, the polypeptide comprises from N- to C-terminus: the Fc polypeptide; a polypeptide linker; and the IDUA enzyme, the IDUA variant amino acid sequence, or the catalytically active fragment thereof.

In certain embodiments, the Fc polypeptide comprises T366S, L368A, and Y407V substitutions, according to EU numbering.

In certain embodiments, the Fc polypeptide comprises substitutions of Ala at position 234 and Ala at position 235; Ala at position 234, Ala at position 235 and Gly at position 329; or Ala at position 234, Ala at position 235 and Ser at position 329, according to EU numbering.

In certain embodiments, the polypeptide comprises an amino acid sequence having at least 95% or 100% identity to any one of SEQ ID NOS:50-69 and 83-92.

Certain embodiments provide a protein comprising 1) a polypeptide comprising an Fc polypeptide that is linked to an alpha-L-iduronidase (IDUA) amino acid sequence, an IDUA variant amino acid sequence, or a catalytically active fragment thereof, wherein the Fc polypeptide comprises a sequence having at least 90% identity to SEQ ID NO: 12 and contains one or more modifications that promote its heterodimerization to another Fc polypeptide; and 2) the other Fc polypeptide.

Certain embodiments provide a pharmaceutical composition comprising a fusion protein as described herein or a polypeptide as described herein and a pharmaceutically acceptable carrier and/or excipient.

Certain embodiments provide a polynucleotide comprising a nucleic acid sequence encoding a polypeptide as described herein (e.g., an IDUA-Fc fusion polypeptide as described herein).

Certain embodiments provide a vector comprising a polynucleotide as described herein.

Certain embodiments provide a host cell comprising a polynucleotide as described herein or a vector as described herein. In certain embodiments, the host cell further comprises a polynucleotide comprising a nucleic acid sequence encoding another polypeptide described herein (e.g., the other Fc polypeptide, such as a TfR-binding modified Fc polypeptide).

Certain embodiments provide a method for producing a polypeptide comprising an Fc polypeptide that is linked to an IDUA amino acid sequence, IDUA variant amino acid sequence, or catalytically active fragment thereof, comprising culturing a host cell under conditions in which the polypeptide encoded by a polynucleotide as described herein is expressed.

Certain embodiments provide a pair of polynucleotides comprising a first nucleic acid sequence encoding a first Fc polypeptide linked to an IDUA amino acid sequence, IDUA variant amino acid sequence, or catalytically active fragment thereof, and a second nucleic acid sequence encoding a second Fc polypeptide, as described herein.

Certain embodiments provide one or more vectors comprising the pair of polynucleotides as described herein. For example, certain embodiments provide a single vector comprising the pair of polynucleotides. Other embodiments provide two vectors, wherein the first vector comprises the first polynucleotide from the pair and the second vector comprises the second polynucleotide from the pair.

Certain embodiments provide a host cell comprising the pair of polynucleotides as described herein, or the one or more vectors as described herein.

Certain embodiments provide a method for producing a protein comprising a first Fc polypeptide linked to an IDUA amino acid sequence, IDUA variant amino acid sequence, or catalytically active fragment thereof, and a second Fc polypeptide, comprising culturing a host cell under conditions in which a pair of polynucleotides as described herein are expressed.

Certain embodiments provide a method of treating MPS I, the method comprising administering a protein as described herein or a polypeptide as described herein to a patient in need thereof. In certain embodiments, a therapeutically effective amount of the protein or polypeptide is administered.

Certain embodiments provide a protein as described herein or a polypeptide as described herein for use in treating MPS I in a patient in need thereof.

Certain embodiments provide the use of a protein as described herein or a polypeptide as described herein in the preparation of a medicament for treating MPS I in a patient in need thereof.

Certain embodiments provide a method of decreasing the accumulation of a toxic metabolic product in a patient having MPS I, the method comprising administering a protein as described herein or a polypeptide as described herein to the patient. In certain embodiments, an effective amount (e.g., a therapeutically effective amount) of the protein or polypeptide is administered.

Certain embodiments provide a protein as described herein or a polypeptide as described herein for use in decreasing the accumulation of a toxic metabolic product in a patient having MPS I.

Certain embodiments provide the use of a protein as described herein or a polypeptide as described herein in the preparation of a medicament for decreasing the accumulation of a toxic metabolic product in a patient having MPS I.

In certain embodiments, the toxic metabolic product comprises heparan sulfate-derived oligosaccharides or dermatan sulfate-derived oligosaccharides.

### BRIEF DESCRIPTION OF THE FIGURES

FIGS. 1A-1B. Illustration of exemplary ETV:IDUA fusion proteins, wherein the IDUA enzyme is fused to (FIG. 1A) the N-terminus of an Fc polypeptide; or to (FIG. 1B) the C-terminus of an Fc polypeptide.

FIG. 2. In vitro evaluation of enzymatic activity of IDUA-Fc fusion proteins and Aldurazyme (laronidase).

FIG. 3. Evaluation of cellular activity of ETV:IDUA Fusion 1 as compared to laronidase in fibroblasts from Hurler (MPS I) patients and healthy controls using a LCMS quantification of heparan sulfate and dermatan sulfate.

FIGS. 4A-4C. Evaluation of (FIG. 4A) ETV:IDUA Fusion 3 serum PK, (FIG. 4B) ETV:IDUA Fusion 4 serum PK, and (FIG. 4C) ETV:IDUA Fusion 6 serum PK in TfR knock in mice (“TfR^{mu/hu}KI”) FIGS. 5A-5C. Evaluation of (FIG. 4A) ETV:IDUA Fusion 3 brain PK, (FIG. 4B) ETV:IDUA Fusion 4 brain PK, and (FIG. 4C) ETV:IDUA Fusion 6 brain PK in TfR knock in mice (“TfR^{mu/hu}KI”).

FIGS. 6A-6D. Evaluation of pharmacodynamic response (total GAG levels) in CSF (FIG. 6A), brain (FIG. 6B), liver (FIG. 6C), and urine (FIG. 6D) in a comparative study carried out in healthy and disease mice models of MPS I. The healthy mouse model is represented by TfR knock in mice (“TfR^{mu/hu}”) and the disease mouse model is represented by TfR knock in mice in which the gene for IDUA has been knocked out (“IDUA KO; TfR^{mu/hu}”) Graphs display mean SEM and p values: one-way ANOVA Dunnett's multiple comparison test; ** p≤0.01, *** p≤0.001, and **** p≤0.0001.

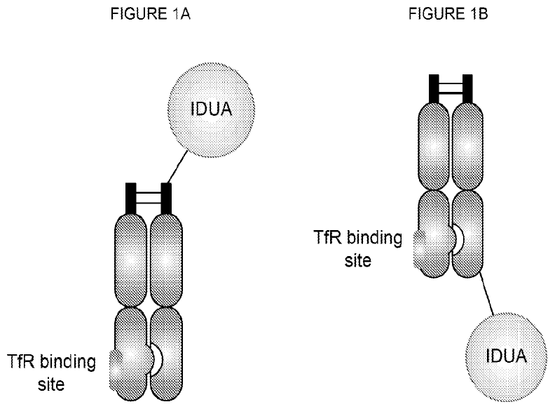

### DETAILED DESCRIPTION

There is currently a need for new therapeutics for the treatment of MPS I, specifically therapeutics that treat severe MPS I having a neurocognitive phenotype. Described herein is a specific enzyme replacement therapy termed ETV:IDUA, which has the capability of crossing the BBB and treating both the peripheral and CNS manifestations of MPS I. As used herein, the term “ETV:IDUA” refers to a protein (e.g., a dimeric protein) that is capable of being transported across the BBB and comprises a first Fc polypeptide linked (e.g., fused) to an IDUA enzyme, an IDUA enzyme variant, or a catalytically active fragment thereof; and a second Fc polypeptide.

### Protein Molecules Comprising an IDUA Enzyme-Fc Fusion Polypeptide

As described herein, certain embodiments provide a protein molecule comprising an IDUA enzyme-Fc fusion polypeptide. An IDUA enzyme incorporated into the protein is catalytically active, i.e., it retains the enzymatic activity. In some aspects, a protein described herein comprises: (a) a first Fc polypeptide, which may contain modifications (e.g., one or more modifications that promote heterodimerization) or may be a wild-type Fc polypeptide; and an IDUA enzyme; and (b) a second Fc polypeptide, which may contain modifications (e.g., one or more modifications that promote heterodimerization) or may be a wild-type Fc polypeptide; and optionally an IDUA enzyme, wherein the first and/or second Fc polypeptide comprises modifications that result in binding to a blood-brain barrier (BBB) receptor, e.g., a transferrin receptor (TfR).

In some embodiments, a protein as described herein comprises a full length IDUA wild-type sequence. In some embodiments, a protein as described herein comprises a mature IDUA wild-type sequence. As described herein, a number of polymorphisms have been reported in the wild-type IDUA protein sequence. For example, the IDUA enzyme may comprise an H or a Q at position 33; and/or may comprise an A or a T at position 622, according to EU numbering. In some embodiments, a protein as described herein comprises a catalytically active fragment or a variant of a wild-type IDUA sequence. For example, the IDUA enzyme may comprise an E at position 27, accordingly to EU numbering, or the amino acid may be absent. Other IDUA enzyme truncations are also described herein. Thus, in some embodiments, the IDUA amino acid sequence comprises an amino acid sequence having at least 80%, at least 85%, at least 90%, at least 95% identity, at least 96% identity, at least 97% identity, at least 98% identity, or at least 99% identity to the amino acid sequence of any one of SEQ ID NOS:39-49, 78-82 and 99, or comprises the amino acid sequence of SEQ ID NOS:39-49, 78-82 and 99. In some embodiments, the IDUA amino acid sequence comprises an amino acid sequence having at least 80%, at least 85%, at least 90%, at least 95% identity, at least 96% identity, at least 97% identity, at least 98% identity, or at least 99% identity to the amino acid sequence of any one of SEQ ID NOS:39, 40, 45, 78, and 99, or comprises the amino acid sequence of any one of SEQ ID NOS:39, 40, 45, 78, and 99. In certain embodiments, within such sequences X₁ is H. In certain embodiments, within such sequences X₁ is Q. In certain embodiments, within such sequences X₂ is A. In certain embodiments, within such sequences X₂ is T. In certain embodiments, within such sequences X₃ is E. In certain embodiments, within such sequences X₃ is absent. In some embodiments, the IDUA amino acid sequence comprises an amino acid sequence having at least 80%, at least 85%, at least 90%, at least 95% identity, at least 96% identity, at least 97% identity, at least 98% identity, or at least 99% identity to the amino acid sequence of any one of SEQ ID NOS: 41-44, or comprises the amino acid sequence of any one of SEQ ID NOS: 41-44. In some embodiments, the IDUA amino acid sequence comprises an amino acid sequence having at least 80%, at least 85%, at least 90%, at least 95% identity, at least 96% identity, at least 97% identity, at least 98% identity, or at least 99% identity to the amino acid sequence of any one of SEQ ID NOS: 46-49, or comprises the amino acid sequence of any one of SEQ ID NOS: 46-49. In some embodiments, the IDUA amino acid sequence comprises an amino acid sequence having at least 80%, at least 85%, at least 90%, at least 95% identity, at least 96% identity, at least 97% identity, at least 98% identity, or at least 99% identity to the amino acid sequence of any one of SEQ ID NOS: 79-82, or comprises the amino acid sequence of any one of SEQ ID NOS: 79-82.

As discussed above, in some embodiments, the IDUA enzyme is a variant or a catalytically active fragment of an IDUA protein (e.g., comprises an IDUA amino acid sequence described herein). In some embodiments, a catalytically active variant or fragment of an IDUA enzyme has at least 50%, at least 55%, at least 60%, at least 65%, at least 70%, at least 75%, at least 80%, at least 85%, at least 90%, at least 95%, or greater of the activity of the wild-type IDUA enzyme.

In some embodiments, an IDUA enzyme, or a catalytically active variant or fragment thereof, that is present in a protein described herein, retains at least 25% of its activity compared to its activity when not joined to an Fc polypeptide or a TfR-binding Fc polypeptide. In some embodiments, an IDUA enzyme, or a catalytically active variant or fragment thereof, retains at least 10%, or at least 15%, 20%, 25%, 30%, 35%, 40%, 45%, 50%, 55%, 60%, 65%, 70%, 75%, 80%, 85%, 90%, or 95%, of its activity compared to its activity when not joined to an Fc polypeptide or a TfR-binding Fc polypeptide. In some embodiments, an IDUA enzyme, or a catalytically active variant or fragment thereof, retains at least 80%, 85%, 90%, or 95% of its activity compared to its activity when not joined to an Fc polypeptide or a TfR-binding Fc polypeptide. In some embodiments, fusion to an Fc polypeptide does not decrease the activity of the IDUA enzyme, or catalytically active variant or fragment thereof. In some embodiments, fusion to a TfR-binding Fc polypeptide does not decrease the activity of the IDUA enzyme.

### Fc Polypeptide Modifications

An Fc polypeptide incorporated in a fusion protein described herein may comprise certain modifications. For example, an Fc polypeptide may comprise modifications that result in binding to a blood-brain barrier (BBB) receptor, e.g., a transferrin receptor (TfR). Additionally, an Fc polypeptide may comprise other modifications, such as modifications that promote heterodimerization, increase serum stability or serum half-life, modulate effector function, influence glycosylation, and/or reduce immunogenicity in humans. Thus, in certain embodiments, a fusion protein described herein comprises two Fc polypeptides, wherein one Fc is a wild-type Fc polypeptide, e.g., a human IgG1 Fc polypeptide; and the other Fc is modified to bind to a blood-brain barrier (BBB) receptor, e.g., transferrin receptor (TfR), and optionally further comprises one or more additional modifications. In certain other embodiments, both Fc polypeptides each comprise independently selected modifications (e.g., a modification described herein). For example, in certain embodiments, a fusion protein described herein comprises two Fc polypeptides, wherein one Fc is not modified to bind to a BBB receptor but comprises one or more other modifications described herein; and the other Fc is modified to bind to a blood-brain barrier (BBB) receptor, e.g., transferrin receptor (TfR), and optionally further comprises one or more additional modifications. In certain other embodiments, a fusion protein described herein comprises two Fc polypeptides, wherein both Fc polypeptides are modified to bind to a blood-brain barrier (BBB) receptor, e.g., transferrin receptor (TfR), and optionally further comprise one or more additional modifications.

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein.

A modified (e.g., enhancing heterodimerization and/or BBB receptor-binding) Fc polypeptide present in a fusion protein described herein can have at least 70% identity, at least 75% identity, at least 80% identity, at least 85% identity, at least 90% identity, at least 95% identity, at least 96% identity, at least 97% identity, at least 98% identity, or at least 99% identity to a native Fc region sequence or a fragment thereof, e.g., a fragment of at least 50 amino acids or at least 100 amino acids, or greater in length. In some embodiments, the native Fc amino acid sequence is the Fc region sequence of SEQ ID NO:1. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 1-110 of SEQ ID NO:1, or to amino acids 111-217 of SEQ ID NO:1, or a fragment thereof, e.g., a fragment of at least 50 amino acids or at least 100 amino acids, or greater in length.

In some embodiments, a modified (e.g., enhancing heterodimerization and/or BBB receptor-binding) Fc polypeptide comprises at least 50 amino acids, or at least 60, 65, 70, 75, 80, 85, 90, or 95 or more, or at least 100 amino acids, or more, that correspond to a native Fc region amino acid sequence. In some embodiments, the modified Fc polypeptide comprises at least 25 contiguous amino acids, or at least 30, 35, 40, or 45 contiguous amino acids, or 50 contiguous amino acids, or at least 60, 65, 70, 75, 80 85, 90, or 95 or more contiguous amino acids, or 100 or more contiguous amino acids, that correspond to a native Fc region amino acid sequence, such as SEQ ID NO:1.

### Modifications for Blood-Brain Barrier (BBB) Receptor Binding

In some aspects, provided herein are fusion proteins that are capable of being transported across the blood-brain barrier (BBB). Such a protein comprises a modified Fc polypeptide that binds to a BBB receptor. BBB receptors are expressed on BBB endothelia, as well as other cell and tissue types. In some embodiments, the BBB receptor is a transferrin receptor (TfR).

In some embodiments a fusion protein described herein specifically binds to TfR. In some embodiments a fusion protein described herein specifically binds to TfR with an affinity of from about 50 nM to about 500 nM. In some embodiments, the protein binds (e.g., specifically binds) to a TfR with an affinity of about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 nM. In some embodiments, the protein binds to a TfR with an affinity of from about 100 to about 500 nM. In some embodiments, the protein binds to a TfR with an affinity of from about 100 nM to about 300 nM, or from about 200 nM to about 450 nM. In some embodiments, the protein binds to a TfR with an affinity of about 250 nM. In some embodiments, the protein binds to a TfR with an affinity of from about 150 to about 400 nM, or from about 200 to about 400 nM, or from about 250 nM to about 350 nM, or from about 300 to about 350 nM.

In some embodiments, a modified Fc polypeptide that specifically binds to TfR comprises substitutions in a CH3 domain. In some embodiments, a modified Fc polypeptide comprises a human Ig CH3 domain, such as an IgG CH3 domain, that is modified for TfR-binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 15%, 10%, 9%, 8%, 7%, 6%, 5%, 4%, 3%, 2%, or 1%).

In some embodiments, a modified (e.g., BBB receptor-binding) Fc polypeptide present in a fusion protein described herein comprises substitutions at amino acid positions 384, 386, 387, 388, 389, 413, 415, 416, and 421, according to the EU numbering scheme.

In some embodiments, a modified Fc polypeptide that specifically binds to TfR comprises Ala at position 389, according to EU numbering. In some embodiments, a modified Fc polypeptide that specifically binds to TfR comprises at the following positions, according to EU numbering: Glu at position 380; Ala at position 389; and Asn at position 390. In some embodiments, a modified Fc polypeptide that specifically binds to TfR comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421.

In additional embodiments, the modified Fc polypeptide further comprises one, two, or three substitutions at positions comprising 414, 424, and 426, according to the EU numbering scheme. In some embodiments, position 414 is Lys, Arg, Gly, or Pro; position 424 is Ser, Thr, Glu, or Lys; and/or position 426 is Ser, Trp, or Gly.

In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-217 of SEQ ID NO:23; and comprises the amino acids at EU index positions 380, 384-390 and/or 413-421 of SEQ ID NO:23. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to amino acids 111-216 of SEQ ID NO: 24; and comprises the amino acids at EU index positions 380, 384-390 and/or 413-421 of SEQ ID NO:23 or 24. In some embodiments, the modified Fc polypeptide has at least 70% identity, at least 75% identity, at least 80% identity, at least 85% identity, at least 90% identity, at least 95% identity, at least 96% identity, at least 97% identity, at least 98% identity, or at least 99% identity to SEQ ID NO:23 or 24; and comprises the amino acids at EU index positions 380, 384-390 and/or 413-421 of SEQ ID NO:23 or 24.

In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to SEQ ID NO:23 or 24, and has Ala at position 389, according to EU numbering. In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to SEQ ID NO:23 or 24 and comprises at the following positions, according to EU numbering: Glu at position 380; Ala at position 389; and Asn at position 390. In some embodiments, the modified Fc polypeptide has at least 75% identity, at least 80% identity, at least 85% identity, at least 90% identity, at least 95% identity, at least 96% identity, at least 97% identity, at least 98% identity, or at least 99% identity to SEQ ID NO:23 or 24 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421.

In some embodiments, the modified Fc polypeptide comprises the amino acid sequence of SEQ ID NO:23 or 24.

### Additional Fc Polypeptide Mutations

In some aspects, a fusion protein described herein comprises two Fc polypeptides, wherein one or both Fc polypeptides each comprise independently selected modifications (e.g., a modification described herein). Non-limiting examples of other mutations that can be introduced into one or both Fc polypeptides include, e.g., mutations to increase serum stability or serum half-life, to modulate effector function, to influence glycosylation, to reduce immunogenicity in humans, and/or to provide for knob and hole heterodimerization of the Fc polypeptides. Examples of various modifications that may be included in an Fc polypeptide are described in WO2019/070577, which is incorporated by reference herein in its entirety for all purposes.

In some embodiments, the Fc polypeptides present in the fusion protein each independently have an amino acid sequence identity of at least about 75%, 80%, 85%, 90%, 95%, 96%, 97%, 98%, or 99% to a corresponding wild-type Fc polypeptide (e.g., a human IgG1, IgG2, IgG3, or IgG4 Fc polypeptide).

In some embodiments, the Fc polypeptides present in the fusion protein include knob and hole mutations to promote heterodimer formation and hinder homodimer formation. Generally, the modifications introduce a protuberance (“knob”) at the interface of one polypeptide and a corresponding cavity (“hole”) in the interface of another polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides present in the fusion protein comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of a fusion protein described herein has the T366W knob mutation and the other Fc polypeptide has the Y407V mutation, which is typically accompanied by the T366S and L368A hole mutations. In certain embodiments, the first Fc polypeptide contains the T366S, L368A, and Y407V substitutions and the second Fc polypeptide contains the T366W substitution. In certain other embodiments, the first Fc polypeptide contains the T366W substitution and the second Fc polypeptide contains the T366S, L368A, and Y407V substitutions.

In some embodiments, modifications to enhance serum half-life may be introduced. For example, in some embodiments, one or both Fc polypeptides present in a fusion protein described herein may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, as numbered according to the EU numbering scheme. Alternatively, one or both Fc polypeptides may have an N434S or N434A substitution.

In some embodiments, one or both Fc polypeptides present in a fusion protein described herein may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides present in a fusion protein described herein may also be engineered to contain other modifications for heterodimerization, e.g., electrostatic engineering of contact residues within a CH3-CH3 interface that are naturally charged or hydrophobic patch modifications.

In some embodiments, one or both Fc polypeptides present in a fusion protein described herein may include additional modifications that modulate effector function.

In some embodiments, one or both Fc polypeptides present in a fusion protein described herein may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (LALA) substitutions.

Additional Fc polypeptide mutations that modulate an effector function include, but are not limited to, the following: position 329 may have a mutation in which proline is substituted with a glycine, serine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; L234A, L235A, and P329S of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

In some embodiments, the C-terminal Lys residue is removed in an Fc polypeptide described herein (i.e., the Lys residue at position 447, according to the EU numbering scheme).

### Illustrative Fc Polypeptides Comprising Additional Mutations

As described herein, and by way of non-limiting example, one or both Fc polypeptides present in a fusion protein described herein may comprise additional mutations, including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G or P329S (e.g., L234A and L235A; L234A, L235A, and P329G; or L234A, L235A, and P329S)) as numbered according to the EU numbering scheme), and/or mutations that increase serum stability or serum half-life (e.g., (i) M252Y, S254T, and T256E as numbered with reference to EU numbering, or (ii) N434S with or without M428L as numbered according to the EU numbering scheme). By way of illustration, SEQ ID NOS:9-22, 25-38, 74-77, and 97-98 provide non-limiting examples of modified Fc polypeptides comprising one or more of these additional mutations.

In some embodiments, an Fc polypeptide or a modified Fc polypeptide may have a knob mutation (e.g., T366W as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS:1, 2, 23 and 24. In some embodiments, an Fc polypeptide or a modified Fc polypeptide having the sequence of any one of SEQ ID NOS: 1, 2, 23 and 24 may be modified to have a knob mutation.

In some embodiments, a modified Fc polypeptide comprises a knob mutation (e.g., T366W as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS: 17 and 18. In some embodiments, the modified Fc polypeptide comprises the sequence of any one of SEQ ID NOS: 17 and 18.

In some embodiments, a modified Fc polypeptide comprises a knob mutation (e.g., T366W as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS: 25 and 26. In some embodiments, a modified Fc polypeptide comprises a knob mutation (e.g., T366W as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 25 and 26, and comprises Ala at position 389, according to EU numbering. In some embodiments, a modified Fc polypeptide comprises a knob mutation (e.g., T366W as numbered with reference to EU numbering), has at least 85% identity, at least 90% identity, at least 95% identity, at least 96% identity, at least 97% identity, at least 98% identity, or at least 99% identity to SEQ ID NO:25 or 26, and comprises at the following positions, according to EU numbering: Glu at position 380; Ala at position 389; and Asn at position 390. In some embodiments, a modified Fc polypeptide comprises a knob mutation (e.g., T366W as numbered with reference to EU numbering), has at least 85% identity, at least 90% identity, at least 95% identity, at least 96% identity, at least 97% identity, at least 98% identity, or at least 99% identity to SEQ ID NO:25 or 26, and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 25 and 26.

In some embodiments, an Fc polypeptide or a modified Fc polypeptide may have a knob mutation (e.g., T366W as numbered according to the EU numbering scheme), mutations that modulate effector function (e.g., L234A, L235A, and/or P329G or P329S (e.g., L234A and L235A; L234A, L235A, and P329G; or L234A, L235A, and P329S)) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 2, 23, and 24. In some embodiments, an Fc polypeptide or a modified Fc polypeptide having the sequence of any one of SEQ ID NOS: 1, 2, 23, and 24 may be modified to have a knob mutation and mutations that modulate effector function.

In some embodiments, a modified Fc polypeptide comprises a knob mutation (e.g., T366W as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), and has at least 85% identity, at least 90% identity, at least 95% identity, at least 96% identity, at least 97% identity, at least 98% identity, or at least 99% identity to the sequence of any one of SEQ ID NOS:74-75 and 76-77. In some embodiments, the modified Fc polypeptide comprises the sequence of any one of SEQ ID NOS: 74-75 and 76-77.

In some embodiments, a modified Fc polypeptide comprises a knob mutation (e.g., T366W as numbered with reference to EU numbering) and mutations that modulate effector function (e.g., L234A, L235A, and/or P329G or P329S (e.g., L234A and L235A; L234A, L235A, and P329G; or L234A, L235A, and P329S) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 27-32 and 35-38. In some embodiments, a modified Fc polypeptide comprises a knob mutation (e.g., T366W as numbered with reference to EU numbering) and mutations that modulate effector function (e.g., L234A, L235A, and/or P329G or P329S (e.g., L234A and L235A; L234A, L235A, and P329G; or L234A, L235A, and P329S) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 27-32 and 35-38, and comprises Ala at position 389, according to EU numbering. In some embodiments, a modified Fc polypeptide comprises a knob mutation (e.g., T366W as numbered with reference to EU numbering) and mutations that modulate effector function (e.g., L234A, L235A, and/or P329G or P329S (e.g., L234A and L235A; L234A, L235A, and P329G; or L234A, L235A, and P329S) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 27-32 and 35-38 and comprises at the following positions, according to EU numbering: Glu at position 380; Ala at position 389; and Asn at position 390. In some embodiments, a modified Fc polypeptide comprises a knob mutation (e.g., T366W as numbered with reference to EU numbering) and mutations that modulate effector function (e.g., L234A, L235A, and/or P329G or P329S (e.g., L234A and L235A; L234A, L235A, and P329G; or L234A, L235A, and P329S) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 27-32 and 35-38 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 27-32 and 35-38.

In some embodiments, an Fc polypeptide or a modified Fc polypeptide may have hole mutations (e.g., T366S, L368A, and Y407V as numbered according to the EU numbering scheme) and at least 85% identity, at least 90% identity, at least 95% identity, at least 96% identity, at least 97% identity, at least 98% identity, or at least 99% identity to the sequence of any one of SEQ ID NOS: 1, 2, 23, and 24. In some embodiments, an Fc polypeptide or a modified Fc polypeptide having the sequence of any one of SEQ ID NOS: 1, 2, 23, and 24 may be modified to have hole mutations.

In some embodiments, a modified Fc polypeptide comprises hole mutations (e.g., T366S, L368A, and Y407V as numbered with reference to EU numbering) and has at least 85% identity, at least 90% identity, at least 95% identity, at least 96% identity, at least 97% identity, at least 98% identity, or at least 99% identity to the sequence of any one of SEQ ID NOS: 9 and 10. In some embodiments, the modified Fc polypeptide comprises the sequence of any one of SEQ ID NOS: 9 and 10.

In some embodiments, a modified Fc polypeptide comprises hole mutations (e.g., T366S, L368A, and Y407V as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 33 and 34. In some embodiments, a modified Fc polypeptide comprises hole mutations (e.g., T366S, L368A, and Y407V as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 33 and 34, and comprises Ala at position 389, according to EU numbering. In some embodiments, a modified Fc polypeptide comprises hole mutations (e.g., T366S, L368A, and Y407V as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 33 and 34 and comprises at the following positions, according to EU numbering: Glu at position 380; Ala at position 389; and Asn at position 390. In some embodiments, a modified Fc polypeptide comprises hole mutations (e.g., T366S, L368A, and Y407V as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 33 and 34 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 33 and 34.

In some embodiments, an Fc polypeptide or a modified Fc polypeptide may have hole mutations (e.g., T366S, L368A, and Y407V as numbered according to the EU numbering scheme), mutations that modulate effector function (e.g., L234A, L235A, and/or P329G or P329S (e.g., L234A and L235A; L234A, L235A, and P329G; or L234A, L235A, and P329S)) as numbered according to the EU numbering scheme), and at least 85% identity, at least 90% identity, at least 95% identity, at least 96% identity, at least 97% identity, at least 98% identity, or at least 99% identity to the sequence of any one of SEQ ID NOS: 1, 2, 23 and 24. In some embodiments, an Fc polypeptide or a modified Fc polypeptide having the sequence of any one of SEQ ID NOS: 1, 2, 23, and 24 may be modified to have hole mutations and mutations that modulate effector function.

In some embodiments, a modified Fc polypeptide comprises hole mutations (e.g., T366S, L368A, and Y407V as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), and has at least 85% identity, at least 90% identity, at least 95% identity, at least 96% identity, at least 97% identity, at least 98% identity, or at least 99% identity to the sequence of any one of SEQ ID NOS: 11-16 and 19-22. In some embodiments, the modified Fc polypeptide comprises the sequence of any one of SEQ ID NOS: 11-16 and 19-22.

In some embodiments, a modified Fc polypeptide comprises hole mutations (e.g., T366S, L368A, and Y407V as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 97-98. In some embodiments, a modified Fc polypeptide comprises hole mutations (e.g., T366S, L368A, and Y407V as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 97-98, and comprises Ala at position 389, according to EU numbering. In some embodiments, a modified Fc polypeptide comprises hole mutations (e.g., T366S, L368A, and Y407V as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 97-98 and comprises at the following positions, according to EU numbering: Glu at position 380; Ala at position 389; and Asn at position 390. In some embodiments, a modified Fc polypeptide comprises hole mutations (e.g., T366S, L368A, and Y407V as numbered with reference to EU numbering) and mutations that modulate effector function (e.g., L234A, L235A, and/or P329G or P329S (e.g., L234A and L235A; L234A, L235A, and P329G; or L234A, L235A, and P329S)) as numbered with reference to EU numbering), has at least 85% identity, at least 90% identity, at least 95% identity, at least 96% identity, at least 97% identity, at least 98% identity, or at least 99% identity to the sequence of any one of SEQ ID NOS: 97-98 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the modified Fc polypeptide comprises the sequence of any one of SEQ ID NOS: 97-98.

### FcRn Binding Sites

In certain aspects, modified (e.g., BBB receptor-binding) Fc polypeptides, or Fc polypeptides present in a fusion protein described herein that do not specifically bind to a BBB receptor, can comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart modified Fc polypeptide having the wild-type residues at the mutated positions when assayed under the same conditions). In some embodiments, an FcRn binding site has one or more amino acid residues that are substituted at positions 250-256, 307, 380, 428, and 433-436, according to the EU numbering scheme.

In some embodiments, one or more residues at or near an FcRn binding site are mutated, relative to a native human IgG sequence, to extend serum half-life of the modified polypeptide. In some embodiments, mutations are introduced into one, two, or three of positions 252, 254, and 256. In some embodiments, the mutations are M252Y, S254T, and T256E. In some embodiments, a modified Fc polypeptide further comprises the mutations M252Y, S254T, and T256E. In some embodiments, a modified Fc polypeptide comprises a substitution at one, two, or all three of positions T307, E380, and N434, according to the EU numbering scheme. In some embodiments, the mutations are T307Q and N434A. In some embodiments, a modified Fc polypeptide comprises mutations T307A, E380A, and N434A. In some embodiments, a modified Fc polypeptide comprises substitutions at positions T250 and M428, according to the EU numbering scheme. In some embodiments, the modified Fc polypeptide comprises mutations T250Q and/or M428L. In some embodiments, a modified Fc polypeptide comprises substitutions at positions M428 and N434, according to the EU numbering scheme. In some embodiments, the modified Fc polypeptide comprises mutations M428L and N434S. In some embodiments, a modified Fc polypeptide comprises an N434S or N434A mutation.

### IDUA Enzymes Linked to Fc Polypeptides

In some embodiments, a fusion protein described herein comprises two Fc polypeptides as described herein and one or both of the Fc polypeptides may further comprise a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:5) or a portion thereof (e.g., DKTHTCPPCP; SEQ ID NO:6). In some embodiments, the hinge region is at the N-terminal region of the Fc polypeptide.

In certain embodiments, the N-terminus of the first Fc polypeptide is linked to the IDUA amino acid sequence, IDUA variant amino acid sequence, or a catalytically active fragment thereof. In certain embodiments, the C-terminus of the first Fc polypeptide is linked to the IDUA amino acid sequence, IDUA variant amino acid sequence, or a catalytically active fragment thereof.

In certain embodiments, a fusion protein described herein comprises a single IDUA amino acid sequence, IDUA variant amino acid sequence, or a catalytically active fragment thereof.

In certain other embodiments, a fusion protein as described herein comprises a second IDUA amino acid sequence, IDUA variant amino acid sequence, or a catalytically active fragment thereof. For example, in certain embodiments, the second Fc polypeptide is linked to an IDUA amino acid sequence, an IDUA variant amino acid sequence, or a catalytically active fragment thereof. In certain embodiments, the N-terminus of the second Fc polypeptide is linked to the second IDUA amino acid sequence, IDUA variant amino acid sequence, or a catalytically active fragment thereof. In certain embodiments, the C-terminus of the second Fc polypeptide is linked to the second IDUA amino acid sequence, IDUA variant amino acid sequence, or a catalytically active fragment thereof.

In certain embodiments, the N-terminus of the first Fc polypeptide is linked to a first IDUA amino acid sequence, IDUA variant amino acid sequence, or a catalytically active fragment thereof; and the N-terminus of the second Fc polypeptide is linked to a second IDUA amino acid sequence, IDUA variant amino acid sequence, or a catalytically active fragment thereof.

In certain embodiments, the C-terminus of the first Fc polypeptide is linked to a first IDUA amino acid sequence, IDUA variant amino acid sequence, or a catalytically active fragment thereof, and the C-terminus of the second Fc polypeptide is linked to a second IDUA amino acid sequence, IDUA variant amino acid sequence, or a catalytically active fragment thereof.

In certain embodiments, the N-terminus of the first Fc polypeptide is linked to a first IDUA amino acid sequence, IDUA variant amino acid sequence, or a catalytically active fragment thereof, and the C-terminus of the second Fc polypeptide is linked to a second IDUA amino acid sequence, IDUA variant amino acid sequence, or a catalytically active fragment thereof.

In certain embodiments, the C-terminus of the first Fc polypeptide is linked to a first IDUA amino acid sequence, IDUA variant amino acid sequence, or a catalytically active fragment thereof, and the N-terminus of the second Fc polypeptide is linked to a second IDUA amino acid sequence, IDUA variant amino acid sequence, or a catalytically active fragment thereof.

In some embodiments, an Fc polypeptide is joined to the IDUA enzyme by a linker, e.g., a peptide linker. In some embodiments, the Fc polypeptide is joined to the IDUA enzyme by a peptide bond or by a peptide linker, e.g., is a fusion polypeptide. The peptide linker may be configured such that it allows for the rotation of the IDUA enzyme relative to the Fc polypeptide to which it is joined; and/or is resistant to digestion by proteases. Peptide linkers may contain natural amino acids, unnatural amino acids, or a combination thereof. In some embodiments, the peptide linker may be a flexible linker, e.g., containing amino acids such as Gly, Asn, Ser, Thr, Ala, and the like (e.g., a glycine-rich linker). Such linkers are designed using known parameters and may be of any length and contain any number of repeat units of any length (e.g., repeat units of Gly and Ser residues). For example, the linker may have repeats, such as two, three, four, five, or more Gly₄-Ser (SEQ ID NO:72) repeats or a single Gly₄-Ser (SEQ ID NO:72). In other aspects, the linker may be Gly-Ser. In some embodiments, the peptide linker may include a protease cleavage site, e.g., that is cleavable by an enzyme present in the central nervous system.

In some embodiments, the IDUA enzyme is joined to the N-terminus of the Fc polypeptide, e.g., by a Gly-Ser linker, a Gly₄-Ser linker (SEQ ID NO:72) or a (Gly₄-Ser)₂ linker (SEQ ID NO:73). In some embodiments, the Fc polypeptide may comprise a hinge sequence or partial hinge sequence at the N-terminus that is joined to the linker or that is directly joined to the IDUA enzyme.

In some embodiments, the IDUA enzyme is joined to the C-terminus of the Fc polypeptide, e.g., by a Gly-Ser linker, a Gly₄-Ser linker (SEQ ID NO:72) or a (Gly₄-Ser)₂ linker (SEQ ID NO:73). In some embodiments, the C-terminus of the Fc polypeptide is directly joined to the IDUA enzyme.

In some embodiments, the IDUA enzyme is joined to the Fc polypeptide by a chemical cross-linking agent. Such conjugates can be generated using well-known chemical cross-linking reagents and protocols. For example, there are a large number of chemical cross-linking agents that are known to those skilled in the art and useful for cross-linking the polypeptide with an agent of interest. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including N-hydroxysuccinimide (NHS) or its water soluble analog N-hydroxysulfosuccinimide (sulfo-NHS), succinimidyl 4-(N-maleimidomethyl)cyclohexane-1-carboxylate (SMCC), m-maleimidobenzoyl-N-hydroxysuccinimide ester (MBS); N-succinimidyl (4-iodoacetyl) aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), and succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exist a number of other cross-linking agents including homobifunctional and photoreactive cross-linkers. Disuccinimidyl subcrate (DSS), bismaleimidohexane (BMH) and dimethylpimelimidate. 2HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers.

### Illustrative Protein Molecules Comprising IDUA Enzyme-Fc Fusion Polypeptides

In some aspects, a fusion protein described herein comprises a first Fc polypeptide that is linked to an IDUA enzyme, IDUA enzyme variant, or a catalytically active fragment thereof, and a second Fc polypeptide; wherein the first and/or second Fc polypeptide is a modified Fc that is capable of binding (e.g., specifically binding) to a blood-brain barrier (BBB) receptor, e.g., a transferrin receptor (TfR). In certain embodiments, the second Fc polypeptide forms an Fc dimer with the first Fc polypeptide. In some embodiments, the first Fc polypeptide and/or the second Fc polypeptide does not include an immunoglobulin heavy and/or light chain variable region sequence or an antigen-binding portion thereof. In some aspects, the fusion protein further comprises a second IDUA enzyme, IDUA enzyme variant, or a catalytically active fragment thereof (e.g., which may be linked to the second Fc polypeptide).

In some embodiments, the first Fc polypeptide is a modified Fc polypeptide and/or the second Fc polypeptide is a modified Fc polypeptide (e.g., comprises one or more modifications described herein). For example, in some embodiments, a modified Fc polypeptide contains one or more modifications that promote its heterodimerization to the other Fc polypeptide. In some embodiments, a modified Fc polypeptide contains one or more modifications that reduce effector function. In some embodiments, a modified Fc polypeptide contains one or more modifications that extend serum half-life. In some embodiments, a modified Fc polypeptide comprises one or more modifications that confer binding BBB) receptor, e.g., transferrin receptor (TfR). For example, in certain embodiments, an Fc polypeptide that is capable of binding to a TfR comprises Ala at position 389, according to EU numbering. In some embodiments, an Fc polypeptide that is capable of binding to a TfR receptor comprises at the following positions, according to EU numbering: Glu at position 380; Ala at position 389; and Asn at position 390. In some embodiments, an Fc polypeptide that is capable of binding to a TfR receptor comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, such an Fc polypeptide specifically binds to TfR.

In some embodiments, the first Fc polypeptide is a modified Fc polypeptide. In some embodiments, the second Fc polypeptide is a modified Fc polypeptide. In some embodiments, the first and the second Fc polypeptide are each a modified Fc polypeptide. In some embodiments, the first Fc polypeptide is a modified polypeptide but does not specifically bind to TfR; and the second Fc polypeptide is a modified polypeptide that is capable of specifically binding to TfR, and optionally, further comprises one or more further modifications described herein. In other embodiments, the first Fc polypeptide is a modified polypeptide that is capable of specifically binding to TfR, and optionally, further comprises one or more further modifications described herein; and the second Fc polypeptide is a modified polypeptide but does not specifically binding to TfR. In some embodiments, the first Fc polypeptide is a modified polypeptide that is capable of specifically binding to TfR, and optionally, further comprises one or more further modifications described herein; and the second Fc polypeptide is a modified polypeptide that is capable of specifically binding to TfR, and optionally, further comprises one or more further modifications described herein.

In some embodiments, a fusion protein described herein comprises a first polypeptide chain that comprises a first Fc polypeptide comprising T366S, L368A, and Y407V (hole) substitutions linked to an IDUA enzyme, IDUA enzyme variant, or a catalytically active fragment thereof, and a second polypeptide chain that comprises a second Fc polypeptide that comprises a T366W (knob) substitution, wherein the first and/or second Fc polypeptide is a modified polypeptide that is capable of binding to TfR. In some embodiments, the first Fc polypeptide and/or the second Fc polypeptide further comprises L234A and L235A (LALA) substitutions. In some embodiments, the first Fc polypeptide and/or the second Fc polypeptide further comprises L234A, L235A, and P329G (LALAPG) substitutions or further comprises L234A, L235A, and P329S (LALAPS) substitutions. In some embodiments, the first Fc polypeptide and/or the second Fc polypeptide further comprises M252Y, S254T, and T256E (YTE) substitutions. In some embodiments, the first Fc polypeptide and/or the second Fc polypeptide further comprises: 1) L234A and L235A (LALA) substitutions; L234A, L235A, and P329G (LALAPG) substitutions; or L234A, L235A, and P329S (LALAPS) substitutions; and 2) M252Y, S254T, and T256E (YTE) substitutions. In some embodiments, the first Fc polypeptide and/or the second Fc polypeptide comprises human IgG1 wild-type residues at positions 234, 235, 252, 254, 256, and 366.

In some embodiments, the second Fc polypeptide is a modified polypeptide that is capable of binding to TfR. In some embodiments, the first Fc polypeptide linked to an IDUA enzyme, IDUA enzyme variant, or a catalytically active fragment thereof is not modified to bind to TfR. In some embodiments, the second Fc polypeptide comprises the knob, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:25-32. In some embodiments, the second Fc polypeptide comprises the knob, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:25-32, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 25-32 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 25-32 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421; or comprises the sequence of any one of SEQ ID NOS: 25-32. In some embodiments, the first Fc polypeptide comprises the hole, LALA/LALAPG/LALAPS, and/or YTE mutations, and has at least 85% identity, at least 90% identity, at least 95% identity, at least 96% identity, at least 97% identity, at least 98% identity, or at least 99% identity to any one of SEQ ID NOS:9-16; or comprises the sequence of any one of SEQ ID NOS:9-16. In some embodiments, the second Fc polypeptide comprises any one of SEQ ID NOS:25-32, and the first Fc polypeptide comprises any one of SEQ ID NOS:9-16. In some embodiments, the N-terminus of the first Fc polypeptide and/or the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS: 35-38. In some embodiments, the second Fc polypeptide has at least 85%, at least 90%, at least 95% identity, at least 96% identity, at least 97% identity, at least 98% identity, or at least 99% identity to any one of SEQ ID NOS: 35-38, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 35-38 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 35-38 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS:35-38. In some embodiments, the first Fc polypeptide has at least 85%, at least 90%, at least 95% identity, at least 96% identity, at least 97% identity, at least 98% identity, or at least 99% identity to any one of SEQ ID NOS: 19-22, or comprises the sequence of any one of SEQ ID NOS: 19-22.

In some embodiments, the second Fc polypeptide is not modified to bind to TfR. In some embodiments, the first Fc polypeptide linked to an IDUA enzyme, IDUA enzyme variant, or a catalytically active fragment thereof is a modified polypeptide that is capable of binding to TfR. In some embodiments, the second Fc polypeptide comprises the knob, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS: 17-18 and 74-75; or comprises the sequence of any one of SEQ ID NOS: 17-18 and 74-75. In some embodiments, the first Fc polypeptide comprises the hole, LALA/LALAPG/LALAPS, and/or YTE mutations, and has at least 85% identity, at least 90% identity, at least 95% identity, at least 96% identity, at least 97% identity, at least 98% identity or at least 99% identity to any one of SEQ ID NOS:33-34 and 97-98. In some embodiments, the first Fc polypeptide comprises the hole, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:33-34 and 97-98, and comprises Ala at position 389, according to EU numbering. In some embodiments, the first Fc polypeptide comprises the hole, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:33-34 and 97-98, and comprises at the following positions, according to EU numbering: Glu at position 380; Ala at position 389; and Asn at position 390. In some embodiments, the first Fc polypeptide comprises the hole, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:33-34 and 97-98, and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS: 33-34 and 97-98. In some embodiments, the second Fc polypeptide comprises any one of SEQ ID NOS:17-18 and 74-75, and the first Fc polypeptide comprises any one of SEQ ID NOS: 33-34 and 97-98. In some embodiments, the N-terminus of the first Fc polypeptide and/or the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS: 76-77; or comprises the sequence of any one of SEQ ID NOS:76-77.

In some embodiments, a fusion protein described herein comprises a first polypeptide chain that comprises a first Fc polypeptide comprising a T366W (knob) substitution linked to an IDUA enzyme, IDUA enzyme variant, or a catalytically active fragment thereof; and a second polypeptide chain that comprises a second Fc polypeptide that comprises T366S, L368A, and Y407V (hole) substitutions, wherein the first and/or second Fc polypeptide is a modified polypeptide that is capable of binding to TfR. In some embodiments, the first Fc polypeptide and/or the second Fc polypeptide further comprises L234A and L235A (LALA) substitutions. In some embodiments, the first Fc polypeptide and/or the second Fc polypeptide further comprises L234A, L235A, and P329G (LALAPG) substitutions or further comprises L234A, L235A, and P329S (LALAPS) substitutions. In some embodiments, the first Fc polypeptide and/or the second Fc polypeptide further comprises M252Y, S254T, and T256E (YTE) substitutions. In some embodiments, the first Fc polypeptide and/or the second Fc polypeptide further comprises: 1) L234A and L235A (LALA) substitutions; L234A, L235A, and P329G (LALAPG) substitutions; or L234A, L235A, and P329S (LALAPS) substitutions; and 2) M252Y, S254T, and T256E (YTE) substitutions. In some embodiments, the first Fc polypeptide and/or the second Fc polypeptide comprises human IgG1 wild-type residues at positions 234, 235, 252, 254, 256, and 366.

In some embodiments, the second Fc polypeptide is a modified polypeptide that is capable of binding to TfR. In some embodiments, the first Fc polypeptide linked to an IDUA enzyme, IDUA enzyme variant, or a catalytically active fragment thereof is not modified to bind to TfR. In some embodiments, the second Fc polypeptide comprises the hole, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:33-34 and 97-98. In some embodiments, the second Fc polypeptide comprises the hole, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:33-34 and 97-98, and comprises Ala at position 389, according to EU numbering. In some of the foregoing embodiments, the second Fc polypeptide further comprises at the following positions, according to EU numbering: Glu at position 380 and Asn at position 390. In some of the foregoing embodiments, the second Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the second Fc polypeptide comprises the sequence of any one of SEQ ID NOS:33-34 and 97-98. In some embodiments, the first Fc polypeptide comprises the knob, LALA/LALAPG/LALAPS, and/or YTE mutations and has at least 85% identity, at least 90% identity, at least 95% identity, at least 96% identity, at least 97% identity, at least 98% identity, or at least 99% identity to any one of SEQ ID NOS:17-18 and 74-75; or comprises the sequence of any one of SEQ ID NOS: 17-18 and 74-75. In some embodiments, the second Fc polypeptide comprises any one of SEQ ID NOS: 33-34 and 97-98, and the first Fc polypeptide comprises any one of SEQ ID NOS:17-18 and 74-75. In some embodiments, the N-terminus of the first Fc polypeptide and/or the second Fc polypeptide includes a portion of an IgG1 hinge region (e.g., DKTHTCPPCP; SEQ ID NO:6). In some embodiments, the first Fc polypeptide has at least 85%, at least 90%, at least 95% identity, at least 96% identity, at least 97% identity, at least 98% identity, or at least 99% identity to any one of SEQ ID NOS: 76-77, or comprises the sequence of any one of SEQ ID NOS:76-77.

In some embodiments, the second Fc polypeptide is not modified to bind to TfR. In some embodiments, the first Fc polypeptide linked to an IDUA enzyme, IDUA enzyme variant, or a catalytically active fragment thereof is a modified polypeptide that is capable of binding to TfR. In some embodiments, the second Fc polypeptide comprises the hole, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:9-16; or comprises the sequence of any one of SEQ ID NOS: 9-16. In some embodiments, the first Fc polypeptide comprises the knob, LALA/LALAPG/LALAPS, and/or YTE mutations, and has at least 85% identity, at least 90% identity, at least 95% identity, at least 96% identity, at least 97% identity, at least 98% identity, or at least 99% identity to any one of SEQ ID NOS:25-32. In some embodiments, the first Fc polypeptide comprises the knob, LALA/LALAPG/LALAPS, and/or YTE mutations, has at least 85% identity, at least 90% identity, at least 95% identity, at least 96% identity, at least 97% identity, at least 98% identity, or at least 99% identity to any one of SEQ ID NOS:25-32, and comprises Ala at position 389, according to EU numbering. In some of the foregoing embodiments, the first Fc polypeptide further comprises at the following positions, according to EU numbering: Glu at position 380 and Asn at position 390. In some of the foregoing embodiments, the first Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some embodiments, the first Fc polypeptide comprises the sequence of any one of SEQ ID NOS: 25-32. In some embodiments, the second Fc polypeptide comprises any one of SEQ ID NOS:9-16, and the first Fc polypeptide comprises any one of SEQ ID NOS: 25-32. In some embodiments, the N-terminus of the first Fc polypeptide and/or the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS: 19-22; or comprises the sequence of any one of SEQ ID NOS: 19-22. In some embodiments, the first Fc polypeptide has at least 85%, at least 90%, at least 95% identity, at least 96% identity, at least 97% identity, at least 98% identity, or at least 99% identity to any one of SEQ ID NOS: 35-38; or comprises the sequence of any one of SEQ ID NOS:35-38.

In some embodiments, an IDUA enzyme, present in a fusion protein described herein is linked to a first polypeptide chain that comprises a first Fc polypeptide having at least 85%, at least 90%, at least 95% identity, at least 96% identity, at least 97% identity, at least 98% identity, or at least 99% identity to any one of SEQ ID NOS: 9-16, or comprises the sequence of any one of SEQ ID NOS: 9-16 (e.g., as a fusion polypeptide). In some embodiments, the IDUA enzyme is linked to the first Fc polypeptide by a linker, such as a flexible linker, and/or a hinge region or portion thereof (e.g., DKTHTCPPCP; SEQ ID NO:6). In some embodiments, the N-terminus of the first Fc polypeptide includes a portion of an IgG1 hinge region (e.g., DKTHTCPPCP; SEQ ID NO:6). In some embodiments, the first Fc polypeptide has at least 85%, at least 90%, at least 95% identity, at least 96% identity, at least 97% identity, at least 98% identity, or at least 99% identity to any one of SEQ ID NOS:19-22, or comprises the sequence of any one of SEQ ID NOS: 19-22. In some embodiments, the IDUA enzyme comprises an IDUA sequence having at least 85%, at least 90%, at least 95% identity, at least 96% identity, at least 97% identity, at least 98% identity, or at least 99% identity to any one of SEQ ID NO:39-49, 78-82, and 99, or comprises the sequence of any one of SEQ ID NO:39-49, 78-82, and 99. In some embodiments, the IDUA sequence linked to the first Fc polypeptide has at least 85%, at least 90%, at least 95% identity, at least 96% identity, at least 97% identity, at least 98% identity, or at least 99% identity to any one of SEQ ID NOS:50-69 and 83-92, or comprises the sequence of any one of SEQ ID NOS:50-69 and 83-92. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 25-32. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 25-32, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 25-32 and comprises at the following positions, according to EU numbering: Glu at position 380; Ala at position 389; and Asn at position 390. In some embodiments, the second polypeptide has at least 85% identity, at least 90% identity, at least 95% identity, at least 96% identity, at least 97% identity, at least 98% identity, or at least 99% identity to the sequence of any one of SEQ ID NOS: 25-32 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS: 25-32. In some embodiments, the N-terminus of the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS:35-38. In some embodiments, the second Fc polypeptide has at least 85%, at least 90%, at least 95% identity, at least 96% identity, at least 97% identity, at least 98% identity, or at least 99% identity to any one of SEQ ID NOS:35-38, and comprises Ala at position 389, according to EU numbering. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 35-38 and comprises at the following positions, according to EU numbering: Glu at position 380; Ala at position 389; and Asn at position 390. In some embodiments, the second Fc polypeptide has at least 85% identity, at least 90% identity, at least 95% identity, at least 96% identity, at least 97% identity, at least 98% identity, or at least 99% identity to the sequence of any one of SEQ ID NOS: 35-38 and comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421, or comprises the sequence of any one of SEQ ID NOS:35-38. In some embodiments, a second IDUA enzyme is linked to the second Fc polypeptide by a linker, such as a flexible linker, and/or a hinge region or portion thereof (e.g., DKTHTCPPCP; SEQ ID NO:6). In some embodiments, the second IDUA enzyme comprises an IDUA sequence having at least 85%, at least 90%, at least 95% identity, at least 96% identity, at least 97% identity, at least 98% identity, or at least 99% identity to any one of SEQ ID NO:39-49, 78-82, and 99, or comprises the sequence of any one of SEQ ID NO:39-49, 78-82, and 99. In some embodiments, the second IDUA sequence linked to the second Fc polypeptide has at least 85%, at least 90%, at least 95% identity, at least 96% identity, at least 97% identity, at least 98% identity, or at least 99% identity to any one of SEQ ID NOS:101-103, or comprises the sequence of any one of SEQ ID NOS:101-103.

In some embodiments, the fusion protein comprises a first Fc polypeptide linked to the IDUA amino acid sequence comprising the amino acid sequence of any one of SEQ ID NOS: 50-65 and 83-92; and a second Fc polypeptide comprising the amino acid sequence of any one of SEQ ID NOS: 35-38.

In some embodiments, the fusion protein comprises a first Fc polypeptide linked to the IDUA amino acid sequence comprising the amino acid sequence of any one of SEQ ID NOS: 50-57 and 83-86; and a second Fc polypeptide comprising the amino acid sequence of any one of SEQ ID NOS: 35-36.

In some embodiments, the fusion protein comprises a first Fc polypeptide linked to an IDUA amino acid sequence comprising the amino acid sequence of any one of SEQ ID NOS:50-53, and a second Fc polypeptide comprising the amino acid sequence of any one of SEQ ID NO:35-36.

In some embodiments, the fusion protein comprises a first Fc polypeptide linked to an IDUA amino acid sequence comprising the amino acid sequence of any one of SEQ ID NOS:50-51, and a second Fc polypeptide comprising the amino acid sequence of any one of SEQ ID NO:35-36.

In some embodiments, the fusion protein comprises a first Fc polypeptide linked to an IDUA amino acid sequence comprising the amino acid sequence of SEQ ID NO:51, and a second Fc polypeptide comprising the amino acid sequence of SEQ ID NO:36.

In some embodiments, the fusion protein comprises a first Fc polypeptide linked to an IDUA amino acid sequence comprising the amino acid sequence of any one of SEQ ID NOS:54-57, and a second Fc polypeptide comprising the amino acid sequence of any one of SEQ ID NO:35-36.

In some embodiments, the fusion protein comprises a first Fc polypeptide linked to an IDUA amino acid sequence comprising the amino acid sequence of any one of SEQ ID NOS:83-86, and a second Fc polypeptide comprising the amino acid sequence of any one of SEQ ID NO:35-36.

In some embodiments, the fusion protein comprises a first Fc polypeptide linked to an IDUA amino acid sequence comprising the amino acid sequence of any one of SEQ ID NOS:83-84, and a second Fc polypeptide comprising the amino acid sequence of any one of SEQ ID NO:35-36.

In some embodiments, the fusion protein comprises a first Fc polypeptide linked to an IDUA amino acid sequence comprising the amino acid sequence of SEQ ID NO:84, and a second Fc polypeptide comprising the amino acid sequence of SEQ ID NO:36.

In some embodiments, the fusion protein comprises a first Fc polypeptide linked to the IDUA amino acid sequence comprising the amino acid sequence of any one of SEQ ID NOS: 58-65 and 87-92; and a second Fc polypeptide comprising the amino acid sequence of any one of SEQ ID NOS: 37-38.

In some embodiments, the fusion protein comprises a first Fc polypeptide linked to an IDUA amino acid sequence comprising the amino acid sequence of any one of SEQ ID NOS:58-61, and a second Fc polypeptide comprising the amino acid sequence of any one of SEQ ID NOS:37-38.

In some embodiments, the fusion protein comprises a first Fc polypeptide linked to an IDUA amino acid sequence comprising the amino acid sequence of any one of SEQ ID NOS:58-59, and a second Fc polypeptide comprising the amino acid sequence of any one of SEQ ID NOS:37-38.

In some embodiments, the fusion protein comprises a first Fc polypeptide linked to an IDUA amino acid sequence comprising the amino acid sequence of SEQ ID NO:59, and a second Fc polypeptide comprising the amino acid sequence of SEQ ID NO:38.

In some embodiments, the fusion protein comprises a first Fc polypeptide linked to an IDUA amino acid sequence comprising the amino acid sequence of any one of SEQ ID NOS:60-61, and a second Fc polypeptide comprising the amino acid sequence of any one of SEQ ID NOS:37-38.

In some embodiments, the fusion protein comprises a first Fc polypeptide linked to an IDUA amino acid sequence comprising the amino acid sequence of SEQ ID NOS:61, and a second Fc polypeptide comprising the amino acid sequence of SEQ ID NO:38.

In some embodiments, the fusion protein comprises a first Fc polypeptide linked to an IDUA amino acid sequence comprising the amino acid sequence of any one of SEQ ID NOS:62-65, and a second Fc polypeptide comprising the amino acid sequence of any one of SEQ ID NO:37-38.

In some embodiments, the fusion protein comprises a first Fc polypeptide linked to an IDUA amino acid sequence comprising the amino acid sequence of any one of SEQ ID NOS:64-65, and a second Fc polypeptide comprising the amino acid sequence of any one of SEQ ID NO:37-38.

In some embodiments, the fusion protein comprises a first Fc polypeptide linked to an IDUA amino acid sequence comprising the amino acid sequence of SEQ ID NOS:65, and a second Fc polypeptide comprising the amino acid sequence of SEQ ID NO:38.

In some embodiments, the fusion protein comprises a first Fc polypeptide linked to an IDUA amino acid sequence comprising the amino acid sequence of any one of SEQ ID NOS:87-90, and a second Fc polypeptide comprising the amino acid sequence of any one of SEQ ID NO:37-38.

In some embodiments, the fusion protein comprises a first Fc polypeptide linked to an IDUA amino acid sequence comprising the amino acid sequence of any one of SEQ ID NOS:89-90, and a second Fc polypeptide comprising the amino acid sequence of any one of SEQ ID NO:37-38.

In some embodiments, the fusion protein comprises a first Fc polypeptide linked to an IDUA amino acid sequence comprising the amino acid sequence of SEQ ID NO: 90, and a second Fc polypeptide comprising the amino acid sequence of SEQ ID NO:38.

In some embodiments, the fusion protein comprises a first Fc polypeptide linked to an IDUA amino acid sequence comprising the amino acid sequence of any one of SEQ ID NOS:91-92, and a second Fc polypeptide comprising the amino acid sequence of any one of SEQ ID NO:37-38.

In some embodiments, the fusion protein comprises a first Fc polypeptide linked to an IDUA amino acid sequence comprising the amino acid sequence of SEQ ID NO:92, and a second Fc polypeptide comprising the amino acid sequence of SEQ ID NO:38.

In some embodiments, the fusion protein comprises a first Fc polypeptide linked to a first IDUA amino acid sequence comprising the amino acid sequence of any one of SEQ ID NOS:50-51; a second Fc polypeptide linked to a second IDUA amino acid sequence comprising the amino acid sequence of any one of SEQ ID NOS:101 or 102.

In some embodiments, the fusion protein comprises a first Fc polypeptide linked to a first IDUA amino acid sequence comprising the amino acid sequence of SEQ ID NO:51; a second Fc polypeptide linked to a second IDUA amino acid sequence comprising the amino acid sequence of any one of SEQ ID NOS:102.

In some embodiments, the fusion protein comprises a first Fc polypeptide linked to the IDUA amino acid sequence comprising the amino acid sequence of any one of SEQ ID NOS: 66-69; and a second Fc polypeptide comprising the amino acid sequence of any one of SEQ ID NOS: 35-38.

In some embodiments, the fusion protein comprises a first Fc polypeptide linked to the IDUA amino acid sequence comprising the amino acid sequence of any one of SEQ ID NOS: 66-67; and a second Fc polypeptide comprising the amino acid sequence of any one of SEQ ID NOS: 35-36.

In some embodiments, the fusion protein comprises a first Fc polypeptide linked to the IDUA amino acid sequence comprising the amino acid sequence of SEQ ID NO: 67; and a second Fc polypeptide comprising the amino acid sequence of SEQ ID NO: 35-36.

In some embodiments, the fusion protein comprises a first Fc polypeptide linked to the IDUA amino acid sequence comprising the amino acid sequence of SEQ ID NO: 67; and a second Fc polypeptide comprising the amino acid sequence of SEQ ID NO: 36.

In some embodiments, the fusion protein comprises a first Fc polypeptide linked to the IDUA amino acid sequence comprising the amino acid sequence of any one of SEQ ID NOS: 68-69; and a second Fc polypeptide comprising the amino acid sequence of any one of SEQ ID NOS: 37-38.

In some embodiments, the fusion protein comprises a first Fc polypeptide linked to the IDUA amino acid sequence comprising the amino acid sequence of SEQ ID NO: 68 and a second Fc polypeptide comprising the amino acid sequence of SEQ ID NO: 37-38.

In some embodiments, the fusion protein comprises a first Fc polypeptide linked to the IDUA amino acid sequence comprising the amino acid sequence of SEQ ID NO: 68 and a second Fc polypeptide comprising the amino acid sequence of SEQ ID NO: 38.

In some embodiments, the fusion protein comprises a first Fc polypeptide linked to a first IDUA amino acid sequence comprising the amino acid sequence of SEQ ID NO:67; and a second Fc polypeptide linked to a second IDUA amino acid sequence comprising the amino acid sequence of SEQ ID NO:103.

In some embodiments, an IDUA enzyme, present in a fusion protein described herein is linked to a first polypeptide chain that comprises a first Fc polypeptide having at least 85%, at least 90%, at least 95% identity, at least 96% identity, at least 97% identity, at least 98% identity, or at least 99% identity to any one of SEQ ID NOS: 17-18 and 74-75, or comprises the sequence of any one of SEQ ID NOS: 17-18 and 74-75 (e.g., as a fusion polypeptide). In some embodiments, the first IDUA enzyme is linked to the first Fc polypeptide by a linker, such as a flexible linker, and/or a hinge region or portion thereof (e.g., DKTHTCPPCP; SEQ ID NO:6). In some embodiments, the N-terminus of the first Fc polypeptide includes a portion of an IgG1 hinge region (e.g., DKTHTCPPCP; SEQ ID NO:6). In some embodiments, the first Fc polypeptide has at least 85%, at least 90%, at least 95% identity, at least 96% identity, at least 97% identity, at least 98% identity, or at least 99% identity to any one of SEQ ID NOS:76-77, or comprises the sequence of any one of SEQ ID NOS:76-77. In some embodiments, the IDUA enzyme comprises an IDUA sequence having at least 85%, at least 90%, at least 95% identity, at least 96% identity, at least 97% identity, at least 98% identity, or at least 99% identity to any one of SEQ ID NO: 39-49, 78-82, and 99, or comprises the sequence of any one of SEQ ID NO: 39-49, 78-82, and 99. In some embodiments, the IDUA sequence linked to the first Fc polypeptide has at least 85%, at least 90%, at least 95% identity, at least 96% identity, at least 97% identity, at least 98% identity, or at least 99% identity to any one of SEQ ID NOS:100 and 104, or comprises the sequence of any one of SEQ ID NOS:100 and 104. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 33-34 and 97-98. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 33-34 and 97-98, and comprises Ala at position 389, according to EU numbering. In some of the foregoing embodiments, the second polypeptide further comprises at the following positions, according to EU numbering: Glu at position 380 and Asn at position 390. In some of the foregoing embodiments, the second Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some of the foregoing embodiments, the second Fc polypeptide comprises the sequence of any one of SEQ ID NOS:33-34 and 97-98. In some embodiments, the N-terminus of the second Fc polypeptide includes a portion of an IgG1 hinge region (e.g., DKTHTCPPCP; SEQ ID NO:6). In some embodiments, a second IDUA enzyme is linked to the second Fc polypeptide by a linker, such as a flexible linker, and/or a hinge region or portion thereof (e.g., DKTHTCPPCP; SEQ ID NO:6).

In some embodiments, an IDUA enzyme, present in a fusion protein described herein is linked to a first polypeptide chain that comprises a first Fc polypeptide having at least 85%, at least 90%, at least 95% identity, at least 96% identity, at least 97% identity, at least 98% identity, or at least 99% identity to any one of SEQ ID NOS: 25-32 (e.g., as a fusion polypeptide). In some embodiments, the first Fc polypeptide has at least 85%, at least 90%, at least 95% identity, at least 96% identity, at least 97% identity, at least 98% identity, or at least 99% identity to any one of SEQ ID NOS: 25-32, and comprises Ala at position 389, according to EU numbering. In some of the foregoing embodiments, the first polypeptide further comprises at the following positions, according to EU numbering: Glu at position 380 and Asn at position 390. In some of the foregoing embodiments, the first Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some of the foregoing embodiments, the first Fc polypeptide comprises the sequence of any one of SEQ ID NOS:25-32. In some embodiments, the IDUA enzyme is linked to the first Fc polypeptide by a linker, such as a flexible linker, and/or a hinge region or portion thereof (e.g., DKTHTCPPCP; SEQ ID NO:6). In some embodiments, the N-terminus of the first Fc polypeptide includes a portion of an IgG1 hinge region (e.g., DKTHTCPPCP; SEQ ID NO:6). In some embodiments, the first Fc polypeptide has at least 85%, at least 90%, at least 95% identity, at least 96% identity, at least 97% identity, at least 98% identity, or at least 99% identity to any one of SEQ ID NOS:35-38. In some embodiments, the first Fc polypeptide has at least 85%, at least 90%, at least 95% identity, at least 96% identity, at least 97% identity, at least 98% identity, or at least 99% identity to any one of SEQ ID NOS: 35-38, and comprises Ala at position 389, according to EU numbering. In some of the foregoing embodiments, the first polypeptide further comprises at the following positions, according to EU numbering: Glu at position 380 and Asn at position 390. In some of the foregoing embodiments, the first Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some of the foregoing embodiments, the first Fc polypeptide comprises the sequence of any one of SEQ ID NOS:35-38. In some embodiments, the IDUA enzyme comprises an IDUA sequence having at least 85%, at least 90%, at least 95% identity, at least 96% identity, at least 97% identity, at least 98% identity, or at least 99% identity to any one of SEQ ID NO:39-49, 78-82, and 99 or comprises the sequence of any one of SEQ ID NO:39-49, 78-82, and 99. In some embodiments, the IDUA sequence linked to the first Fc polypeptide has at least 85%, at least 90%, at least 95% identity, at least 96% identity, at least 97% identity, at least 98% identity, or at least 99% identity to any one of SEQ ID NOS:101-103, or comprises the sequence of any one of SEQ ID NOS:101-103. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 9-16, or comprises the sequence of any one of SEQ ID NOS:9-16. In some embodiments, the N-terminus of the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS:19-22, or comprises the sequence of any one of SEQ ID NOS:19-22. In some embodiments, a second IDUA enzyme is linked to the second Fc polypeptide by a linker, such as a flexible linker, and/or a hinge region or portion thereof (e.g., DKTHTCPPCP; SEQ ID NO:6). In some embodiments, the second IDUA enzyme comprises an IDUA sequence having at least 85%, at least 90%, at least 95% identity, at least 96% identity, at least 97% identity, at least 98% identity, or at least 99% identity to any one of SEQ ID NO:39-49, 78-82, and 99, or comprises the sequence of any one of SEQ ID NO:39-49, 78-82, and 99. In some embodiments, the second IDUA sequence linked to the second Fc polypeptide has at least 85%, at least 90%, at least 95% identity, at least 96% identity, at least 97% identity, at least 98% identity, or at least 99% identity to any one of SEQ ID NOS: 50-69 and 83-92, or comprises the sequence of any one of SEQ ID NOS: 50-69 and 83-92.

In some embodiments, an IDUA enzyme, present in a fusion protein described herein is linked to a first polypeptide chain that comprises a first Fc polypeptide having at least 85%, at least 90%, at least 95% identity, at least 96% identity, at least 97% identity, at least 98% identity, or at least 99% identity to any one of SEQ ID NOS: 33-34 and 97-98 (e.g., as a fusion polypeptide). In some embodiments, the first Fc polypeptide has at least 85%, at least 90%, at least 95% identity, at least 96% identity, at least 97% identity, at least 98% identity, or at least 99% identity to any one of SEQ ID NOS: 33-34 and 97-98, and comprises Ala at position 389, according to EU numbering. In some of the foregoing embodiments, the first polypeptide further comprises at the following positions, according to EU numbering: Glu at position 380 and Asn at position 390. In some of the foregoing embodiments, the first Fc polypeptide comprises at the following positions, according to EU numbering: Glu at position 380; Tyr at position 384; Thr at position 386; Glu at position 387; Trp at position 388; Ala at position 389; Asn at position 390; Thr at position 413; Glu at position 415; Glu at position 416; and Phe at position 421. In some of the foregoing embodiments, the first Fc polypeptide comprises the sequence of any one of SEQ ID NOS: 33-34 and 97-98. In some embodiments, the IDUA enzyme is linked to the first Fc polypeptide by a linker, such as a flexible linker, and/or a hinge region or portion thereof (e.g., DKTHTCPPCP; SEQ ID NO:6). In some embodiments, the N-terminus of the first Fc polypeptide includes a portion of an IgG1 hinge region (e.g., DKTHTCPPCP; SEQ ID NO:6). In some embodiments, the IDUA enzyme comprises an IDUA sequence having at least 85%, at least 90%, at least 95% identity, at least 96% identity, at least 97% identity, at least 98% identity, or at least 99% identity to any one of SEQ ID NO:39-49, 78-82, and 99 or comprises the sequence of any one of SEQ ID NO:39-49, 78-82, and 99. In some embodiments, the fusion protein comprises a second Fc polypeptide having at least 85%, at least 90%, at least 95% identity, at least 96% identity, at least 97% identity, at least 98% identity, or at least 99% identity to any one of SEQ ID NOS: 17-18 and 74-75, or comprises the sequence of any one of SEQ ID NOS: 17-18 and 74-75. In some embodiments, the N-terminus of the second Fc polypeptide includes a portion of an IgG1 hinge region (e.g., DKTHTCPPCP; SEQ ID NO:6). In some embodiments, the second Fc polypeptide has at least 85%, at least 90%, at least 95% identity, at least 96% identity, at least 97% identity, at least 98% identity, or at least 99% identity to any one of SEQ ID NOS:76-77, or comprises the sequence of any one of SEQ ID NOS:76-77. In some embodiments, a second IDUA enzyme is linked to the second Fc polypeptide by a linker, such as a flexible linker, and/or a hinge region or portion thereof (e.g., DKTHTCPPCP; SEQ ID NO:6). In some embodiments, the second IDUA enzyme comprises an IDUA sequence having at least 85%, at least 90%, at least 95% identity, at least 96% identity, at least 97% identity, at least 98% identity, or at least 99% identity to any one of SEQ ID NO:39-49, 78-82, and 99, or comprises the sequence of any one of SEQ ID NO:39-49, 78-82, and 99. In some embodiments, the second IDUA sequence linked to the second Fc polypeptide has at least 85%, at least 90%, at least 95% identity, at least 96% identity, at least 97% identity, at least 98% identity, or at least 99% identity to any one of SEQ ID NOS: 100 and 104, or comprises the sequence of any one of SEQ ID NOS: 100 and 104.

In some embodiments, a fusion protein described herein comprises 1) a first Fc polypeptide linked to an IDUA amino acid sequence; and 2) a second Fc polypeptide; wherein each polypeptide consists of an amino acid sequence as recited in a foregoing embodiment.

In some embodiments, a fusion protein described herein comprises 1) a first Fc polypeptide linked to a first IDUA amino acid sequence; and 2) a second Fc polypeptide linked to a second IDUA amino acid sequence; wherein each polypeptide consists of an amino acid sequence as recited in a foregoing embodiment.

Fusion proteins and other compositions described herein may have a range of binding affinities. For example, in some embodiments, a protein has an affinity for a transferrin receptor (TfR), ranging anywhere from about 50 mM to about 500 nM, or from about 100 nM to about 500 nM. In some embodiments, the affinity for TfR ranges from about 50 nM to about 300 nM. In some embodiments, the affinity for TfR ranges from about 100 nM to about 350 nM. In some embodiments, the affinity for TfR ranges from about 150 nM to about 400 nM. In some embodiments, the affinity for TfR ranges from about 200 nM to about 400 nM. In some embodiments, the affinity for TfR ranges from about 200 nM to about 450 nM. In some embodiments, the affinity for TfR is a monovalent affinity.

### Evaluation of Protein Activity

Activity of fusion proteins described herein that comprise IDUA enzymes can be assessed using various assays, including assays that measure activity in vitro using an artificial substrate, such as those described in the Examples section.

In some embodiments, a tissue sample is evaluated. A tissue sample can be evaluated using an assay as described above, except multiple freeze-thaw cycles, e.g., 2, 3, 4, 5, or more, are typically included before the sonication step to ensure that microvesicles are broken open.

Samples that can be evaluated by the assays described herein include brain, liver, kidney, lung, spleen, plasma, serum, cerebrospinal fluid (CSF), and urine. In some embodiments, CSF samples from a patient receiving an enzyme-Fc fusion protein (e.g., IDUA-Fc fusion protein) described herein may be evaluated.

### Nucleic Acids, Vectors, and Host Cells

Polypeptide chains contained in the fusion proteins as described herein are typically prepared using recombinant methods. Accordingly, in some aspects, the present disclosure provides isolated nucleic acids comprising a nucleic acid sequence encoding any of the polypeptide chains comprising Fc polypeptides as described herein, and host cells into which the nucleic acids are introduced that are used to replicate the polypeptide-encoding nucleic acids and/or to express the polypeptides. In some embodiments, the host cell is eukaryotic, e.g., a human cell.

In another aspect, polynucleotides are provided that comprise a nucleotide sequence that encodes one or more of the polypeptide chains described herein. In some embodiments, the polynucleotide encodes one of the polypeptide sequences described here. In some embodiments, the polynucleotide encodes two of the polypeptide sequences described herein. The polynucleotides may be single-stranded or double-stranded. In some embodiments, the polynucleotide is DNA. In particular embodiments, the polynucleotide is cDNA. In some embodiments, the polynucleotide is RNA.

Some embodiments also provide a pair of nucleic acid sequences, wherein each nucleic acid sequence encodes a polypeptide described herein. For example, certain embodiments provide a pair of nucleic acid sequences, wherein a first nucleic acid sequence in the pair encodes a first Fc polypeptide linked to a first IDUA amino acid sequence, IDUA variant amino acid sequence, or a catalytically active fragment thereof; and a second nucleic acid sequence in the pair encodes a second Fc polypeptide, wherein the first and/or second Fc polypeptide is a modified Fc that is capable of binding (e.g., specifically binding) to a blood-brain barrier (BBB) receptor, e.g., a transferrin receptor (TfR).

In some embodiments, the polynucleotide is included within a nucleic acid construct or the pair of polynucleotides is included within one or more nucleic acid constructs. In some embodiments, the construct is a replicable vector. In some embodiments, the vector is selected from a plasmid, a viral vector, a phagemid, a yeast chromosomal vector, and a non-episomal mammalian vector.

In some embodiments, the polynucleotide is operably linked to one or more regulatory nucleotide sequences in an expression construct. In one series of embodiments, the nucleic acid expression constructs are adapted for use as a surface expression library. In some embodiments, the library is adapted for surface expression in yeast. In some embodiments, the library is adapted for surface expression in phage. In another series of embodiments, the nucleic acid expression constructs are adapted for expression of the polypeptide in a system that permits isolation of the polypeptide in milligram or gram quantities. In some embodiments, the system is a mammalian cell expression system. In some embodiments, the system is a yeast cell expression system.

Expression vehicles for production of a recombinant polypeptide include plasmids and other vectors. For instance, suitable vectors include plasmids of the following types: pBR322-derived plasmids, pEMBL-derived plasmids, pEX-derived plasmids, pBTac-derived plasmids, and pUC-derived plasmids for expression in prokaryotic cells, such as E. coli. The pcDNAI/amp, pcDNAI/neo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-1), or Epstein-Barr virus (pHEBo, pREP-derived, and p205) can be used for transient expression of polypeptides in eukaryotic cells. In some embodiments, it may be desirable to express the recombinant polypeptide by the use of a baculovirus expression system. Examples of such baculovirus expression systems include pVL-derived vectors (such as pVL1392, pVL1393, and pVL941), pAcUW-derived vectors (such as pAcUW1), and pBlueBac-derived vectors. Additional expression systems include adenoviral, adeno-associated virus, and other viral expression systems.

Vectors may be transformed into any suitable host cell. In some embodiments, the host cells, e.g., bacteria or yeast cells, may be adapted for use as a surface expression library. In some cells, the vectors are expressed in host cells to express relatively large quantities of the polypeptide. Such host cells include mammalian cells, yeast cells, insect cells, and prokaryotic cells. In some embodiments, the cells are mammalian cells, such as Chinese Hamster Ovary (CHO) cell, baby hamster kidney (BHK) cell, NS0 cell, Y0 cell, HEK293 cell, COS cell, Vero cell, or HeLa cell.

A host cell transfected with an expression vector(s) encoding one or more Fc polypeptide chains as described herein can be cultured under appropriate conditions to allow expression of the one or more polypeptides to occur. The polypeptides may be secreted and isolated from a mixture of cells and medium containing the polypeptides. Alternatively, the polypeptides may be retained in the cytoplasm or in a membrane fraction and the cells harvested, lysed, and the polypeptide isolated using a desired method.

### Therapeutic Methods

A fusion protein as described herein may be used therapeutically to treat MPS I.

Accordingly, certain embodiments provide a method of decreasing the accumulation of a toxic metabolic product (e.g., a heparan sulfate-derived oligosaccharide or a dermatan sulfate-derived oligosaccharide) in a subject having MPS I, the method comprising administering a protein as described herein to the subject.

Certain embodiments provide a protein as described herein for use in decreasing the accumulation of a toxic metabolic product (e.g., a heparan sulfate-derived oligosaccharide or a dermatan sulfate-derived oligosaccharide) in a subject having MPS I.

Certain embodiments provide the use of a protein as described herein in the preparation of a medicament for decreasing the accumulation of a toxic metabolic product (e.g., a heparan sulfate-derived oligosaccharide or a dermatan sulfate-derived oligosaccharide) in a subject having MPS I.

Certain embodiments also provide a method of treating MPS I, comprising administering a protein as described herein to a subject in need thereof.

Certain embodiments provide a protein as described herein for use in treating MPS I in a subject in need thereof.

Certain embodiments provide the use of a protein as described herein in the preparation of a medicament for treating MPS I in a subject in need thereof.

In some embodiments, administration of the protein (e.g., linked to an IDUA enzyme) improves (e.g., increases) Cₘₐₓ of IDUA in the brain as compared to the uptake of IDUA in the absence of being linked to a fusion protein described herein or as compared to the uptake of IDUA linked to a reference protein (e.g., a fusion protein as described herein, which does not have the modifications to the second Fc polypeptide that result in TfR binding).

In some embodiments, Cₘₐₓ of IDUA in the brain is improved (e.g., increased) by at least about 1.1-fold, 1.2-fold, 1.3-fold, 1.4-fold, 1.5-fold, 1.6-fold, 1.7-fold, 1.8-fold, 1.9-fold, 2-fold, 2.2-fold, 2.4-fold, 2.6-fold, 2.8-fold, 3-fold, 4-fold, 5-fold, 6-fold, or more, as compared to the uptake of IDUA in the absence of being linked to a fusion protein described herein or as compared to the uptake of IDUA linked to a reference protein (e.g., a fusion protein as described herein, which does not have the modifications to the second Fc polypeptide that result in TfR binding).

A fusion protein described herein is administered to a subject at a therapeutically effective amount or dose.

In various embodiments, a fusion protein described herein is administered parenterally. In some embodiments, the protein is administered intravenously.

In some parenteral embodiments, a fusion protein as described herein is administered intraperitoneally, intradermally, or intramuscularly. In some embodiments, the fusion protein as described herein is administered intrathecally, such as by epidural administration, or intracerebroventricularly.

### Pharmaceutical Compositions and Kits

In other aspects, pharmaceutical compositions and kits comprising a fusion protein described herein are provided.

### Pharmaceutical Compositions

Guidance for preparing formulations for use in the present disclosure can be found in any number of handbooks for pharmaceutical preparation and formulation that are known to those of skill in the art.

In some embodiments, a pharmaceutical composition comprises a fusion protein as described herein and further comprises one or more pharmaceutically acceptable carriers and/or excipients. A pharmaceutically acceptable carrier includes any solvents, dispersion media, or coatings that are physiologically compatible and that do not interfere with or otherwise inhibit the activity of the active agent.

Dosages and desired drug concentration of pharmaceutical compositions described herein may vary depending on the particular use envisioned.

### Kits

In some embodiments, a kit for use in treating MPS I, comprising a fusion protein as described herein, is provided.

In some embodiments, the kit further comprises one or more additional therapeutic agents. For example, in some embodiments, the kit comprises a fusion protein as described herein and further comprises one or more additional therapeutic agents for use in the treatment of neurological symptoms of MPS I. In some embodiments, the kit further comprises instructional materials containing directions (i.e., protocols) for the practice of the methods described herein (e.g., instructions for using the kit for administering a fusion protein comprising an IDUA enzyme across the blood-brain barrier). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### Certain Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human. In some embodiments, the human is a patient in need of treatment for MPS I. In some embodiments, the patient has one or more signs or symptoms of MPS I.

The term “pharmaceutically acceptable excipient” refers to a non-active pharmaceutical ingredient that is biologically or pharmacologically compatible for use in humans or animals, such as but not limited to a buffer, carrier, or preservative.

The term “administer” refers to a method of delivering agents (e.g., a MPS I therapeutic agent, such as an ETV:IDUA therapy described herein), compounds, or compositions (e.g., pharmaceutical composition) to the desired site of biological action. These methods include, but are not limited to, parenteral delivery, intravenous delivery, intradermal delivery, intramuscular delivery, intrathecal delivery, or intraperitoneal delivery. In one embodiment, the polypeptides described herein are administered intravenously.

As used herein, “treatment” (and grammatical variations thereof such as “treat” or “treating”) refers to clinical intervention to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

A “therapeutically effective amount” of a substance/molecule disclosed herein may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

An “alpha-L-iduronidase,” “iduronidase alpha-L,” “L-iduronidase,” “iduronidase,” or “IDUA” as used herein refers to alpha-L-iduronidase (EC 3.2.1.76), which is an enzyme involved in the lysosomal degradation of glycosaminoglycans, such as dermatan sulfate and heparan sulfate. Mutations in the IDUA gene are associated with MSP I, which results from impaired degradation of heparan sulfate and dermatan sulfate. The term “IDUA” or “IDUA enzyme” as used herein, optionally as a component of a protein that comprises an Fc polypeptide, is catalytically active and encompasses functional variants, including allelic and splice variants, and catalytically active fragments thereof. A sequence of human IDUA is available under UniProt entry P35475 and is encoded by the human IDUA gene at 4p16.3. A full-length sequence is provided as SEQ ID NO:39, which may have an H or Q at position 33 and/or an A or T at position 622, wherein the positions are according to EU numbering. A “mature” IDUA sequence as used herein refers to a form of a polypeptide chain that lacks the signal sequence of the naturally occurring full-length polypeptide chain. An embodiment of an amino acid sequence of a mature human IDUA polypeptide is provided as SEQ ID NO:40, which corresponds to amino acids 27-653 of the full-length human sequence. A “truncated” IDUA sequence as used herein refers to a catalytically active fragment of the naturally occurring full-length polypeptide chain (e.g., SEQ ID NO:45-49, 78-82, and 99 (wherein X₃ is absent)). The structure of human IDUA has been well-characterized. Non-human primate IDUA sequences have also been described, including chimpanzee (e.g., UniProt entry A0A2R9ALZ1 for Pan paniscus (Pygmy chimpanzee) (Bonobo)). A mouse Idua sequence is available under Uniprot entry P48441. An IDUA variant has at least 50%, at least 55%, at least 60%, at least 65%, at least 70%, at least 75%, at least 80%, at least 85%, at least 90%, or at least 95% of the activity of the corresponding wild-type IDUA or fragment thereof, e.g., when assayed under identical conditions. A catalytically active IDUA fragment has at least 50%, at least 55%, at least 60%, at least 65%, at least 70%, at least 75%, at least 80%, at least 85%, at least 90%, or at least 95% of the activity of the corresponding full-length IDUA or variant thereof, e.g., when assayed under identical conditions. A commercially available recombinant form of IDUA is referred to as Aldurazyme or laronidase, with both terms referring to the same recombinant form.

A “transferrin receptor” or “TfR” as used herein refers to transferrin receptor protein 1. The human transferrin receptor 1 polypeptide sequence is set forth in SEQ ID NO:7. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:8.

A “fusion protein” or “[IDUA enzyme]-Fc fusion protein” as used herein refers to a dimeric protein comprising a first Fc polypeptide that is linked (e.g., fused) to an IDUA enzyme, an IDUA enzyme variant, or a catalytically active fragment thereof (i.e., an “[IDUA]-Fc fusion polypeptide”); and a second Fc polypeptide (e.g., that forms an Fc dimer with the first Fc polypeptide). The second Fc polypeptide may also be linked (e.g., fused) to an IDUA enzyme, an IDUA enzyme variant, or a catalytically active fragment thereof. The first Fc polypeptide and/or the second Fc polypeptide may be linked to the IDUA enzyme, IDUA enzyme variant, or catalytically active fragment thereof by a peptide bond or by a polypeptide linker. The first Fc polypeptide and/or the second Fc polypeptide may be a modified Fc polypeptide that contains one or more modifications that promote its heterodimerization to the other Fc polypeptide. The first Fc polypeptide and/or the second Fc polypeptide may be a modified Fc polypeptide that contains one or more modifications that confer binding to a transferrin receptor. The first Fc polypeptide and/or the second Fc polypeptide may be a modified Fc polypeptide that contains one or more modifications that reduce effector function. In certain embodiments, the first Fc polypeptide and the second Fc polypeptide do not have effector function. The first Fc polypeptide and/or the second Fc polypeptide may be a modified Fc polypeptide that contains one or more modifications that extend serum half-life. In certain embodiments, the first Fc polypeptide and/or the second Fc polypeptide do not include an immunoglobulin heavy and/or light chain variable region sequence or an antigen-binding portion thereof. In certain embodiments, the first Fc polypeptide and the second Fc polypeptide do not include an immunoglobulin heavy and/or light chain variable region sequence or an antigen-binding portion thereof.

A “fusion polypeptide” or “[IDUA enzyme]-Fc fusion polypeptide” as used herein refers to an Fc polypeptide that is linked (e.g., fused) to an IDUA enzyme, an IDUA enzyme variant, or a catalytically active fragment thereof. The Fc polypeptide may be linked to the IDUA enzyme, IDUA enzyme variant, or catalytically active fragment thereof by a peptide bond or by a polypeptide linker. The Fc polypeptide may be a modified Fc polypeptide that contains one or more modifications that promote its heterodimerization to another Fc polypeptide. The Fc polypeptide may be a modified Fc polypeptide that contains one or more modifications that confer binding to a transferrin receptor. The Fc polypeptide may be a modified Fc polypeptide that contains one or more modifications that reduce effector function. The Fc polypeptide may be a modified Fc polypeptide that contains one or more modifications that extend serum half-life.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a β2-microglobulin of about 15 kDa in size.

As used herein, an “FcRn binding site” refers to the region of an Fc polypeptide that binds to FcRn. In human IgG, the FcRn binding site, as numbered using the EU index, includes T250, L251, M252, 1253, S254, R255, T256, T307, E380, M428, H433, N434, H435, and Y436. These positions correspond to positions 20 to 26, 77, 150, 198, and 203 to 206 of SEQ ID NO:1.

As used herein, a “native FcRn binding site” refers to a region of an Fc polypeptide that binds to FcRn and that has the same amino acid sequence as the region of a naturally occurring Fc polypeptide that binds to FcRn.

The terms “CH3 domain” and “CH2 domain” as used herein refer to immunoglobulin constant region domain polypeptides. For purposes of this application, a CH3 domain polypeptide refers to the segment of amino acids from about position 341 to about position 447 as numbered according to EU, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:5).

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation. For example, the terms “wild-type,” “native,” and “naturally occurring” with respect to a CH3 or CH2 domain are used herein to refer to a domain that has a sequence that occurs in nature.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region, as measured using a sequence comparison algorithm or by manual alignment and visual inspection. In some embodiments, a sequence that has a specified percent identity relative to a reference sequence differs from the reference sequence by one or more conservative substitutions.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:1, when the residue aligns with the amino acid in SEQ ID NO:1 when optimally aligned to SEQ ID NO:1. The polypeptide that is aligned to the reference sequence need not be the same length as the reference sequence.

The term “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

A “binding affinity” as used herein refers to the strength of the non-covalent interaction between two molecules, e.g., a single binding site on a polypeptide and a target, e.g., transferrin receptor, to which it binds. Thus, for example, the term may refer to 1:1 interactions between a polypeptide and its target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). As used herein, “binding affinity” includes not only formal binding affinities, such as those reflecting 1:1 interactions between a polypeptide and its target, but also apparent affinities for which K_{D}'s are calculated that may reflect avid binding.

As used herein, the term “specifically binds” or “selectively binds” to a target, e.g., TfR, when referring to an engineered TfR-binding polypeptide, TfR-binding peptide, or TfR-binding fusion protein as described herein, refers to a binding reaction whereby the engineered TfR-binding polypeptide, TfR-binding peptide, or TfR-binding fusion protein binds to the target with greater affinity, greater avidity, and/or greater duration than it binds to a structurally different target. In typical embodiments, the engineered TfR-binding polypeptide, TfR-binding peptide, or TfR-binding fusion protein has at least 5-fold, 10-fold, 50-fold, 100-fold, 1,000-fold, 10,000-fold, or greater affinity for a specific target, e.g., TfR, compared to an unrelated target when assayed under the same affinity assay conditions. The term “specific binding,” “specifically binds to,” or “is specific for” a particular target (e.g., TfR), as used herein, can be exhibited, for example, by a molecule having an equilibrium dissociation constant K_{D} for the target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, an engineered TfR-binding polypeptide, TfR-binding peptide, or TfR-binding fusion protein specifically binds to an epitope on TfR that is conserved among species, (e.g., structurally conserved among species), e.g., conserved between non-human primate and human species (e.g., structurally conserved between non-human primate and human species). In some embodiments, an engineered TfR-binding polypeptide, TfR-binding peptide, or TfR-binding fusion protein may bind exclusively to a human TfR.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The terms “antigen-binding portion” and “antigen-binding fragment” are used interchangeably herein and refer to one or more fragments of an antibody that retains the ability to specifically bind to an antigen via its variable region. Examples of antigen-binding fragments include, but are not limited to, a Fab fragment (a monovalent fragment consisting of the VL, VH, CL, and CH1 domains), a F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), a VL (light chain variable region), and a VH (heavy chain variable region).

The following Examples are intended to be non-limiting.

### Example 1: Construction of Fusion Proteins Comprising Alpha-L-Iduronidase (IDUA)

### Design and Cloning

IDUA-Fc fusion proteins were designed that contain (i) a first fusion polypeptide where a human IDUA enzyme is fused to a human IgG1 fragment that includes the Fc region (an “IDUA-Fc fusion polypeptide”), and (ii) a modified human IgG1 fragment, which contains mutations in the Fc region that confer transferrin receptor (TfR) binding (a “modified Fc polypeptide”). Fusion proteins were also designed that contain (i) a first fusion polypeptide where a human IDUA enzyme sequence is fused to a human IgG1 fragment that includes the Fc region (an “IDUA-Fc fusion polypeptide”), and (ii) a second fusion polypeptide where a human IDUA enzyme sequence is fused to a modified human IgG1 fragment which contains mutations in the Fc region that confer TfR binding (an “IDUA-Fc fusion polypeptide that binds TfR”). IDUA-Fc fusion polypeptides were created in which IDUA sequences were fused to the N- and C-terminus of the human IgG1 Fc region. In all constructs, the signal peptide MGWSCIILFLVATATGAYA (SEQ ID NO: 70) was inserted upstream of the fusion to facilitate secretion. The fragment of the human IgG1 Fc region used corresponds to amino acids D104-K330 of the sequence in UniProtKB ID P01857 (positions 221-447, EU numbering, which includes 10 amino acids of the hinge (positions 221-230)). Expression vectors that separately encode (i) the IDUA-Fc fusion polypeptide and (ii) the modified Fc polypeptide were generated and co-transfected into Chinese Hamster Ovary (CHO) cells to generate heterodimeric fusion proteins containing an IDUA enzyme (a “monozyme”). Expression vectors that separately encode (i) the IDUA-Fc fusion polypeptide and (ii) the IDUA-Fc fusion polypeptide that binds TfR were also generated and co-transfected into Chinese Hamster Ovary (CHO) cells to generate heterodimeric fusion proteins containing two IDUA enzymes (a “bizyme”). In some constructs, the IgG1 fragments contained additional mutations to facilitate heterodimerization of the two Fc regions.

An IDUA-Fc fusion polypeptide comprising a mature human IDUA sequence fused to the N-terminus of an IgG1 Fc polypeptide sequence with hole and LALA mutations has the sequence of SEQ ID NO:50 or 51. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An IDUA-Fc fusion polypeptide comprising a mature, human IDUA sequence fused to the N-terminus of an IgG1 Fc polypeptide sequence with hole and LALAPS mutations has the sequence of SEQ ID NO:58 or 59. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An IDUA-Fc fusion polypeptide comprising a mature human IDUA sequence fused to the N-terminus of an IgG1 Fc polypeptide sequence with hole and LALAPS mutations has the sequence of SEQ ID NO:60 or 61. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An IDUA-Fc fusion polypeptide comprising a mature, truncated human IDUA sequence fused to the N-terminus of an IgG1 Fc polypeptide sequence with hole and LALAPS mutations has the sequence of SEQ ID NO:64 or 65. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An IDUA-Fc fusion polypeptide comprising a mature, human IDUA sequence fused to the N-terminus of an IgG1 Fc polypeptide sequence with hole and LALAPS mutations has the sequence of SEQ ID NO:91 or 92. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An IDUA-Fc fusion polypeptide comprising a mature, human IDUA sequence fused to the N-terminus of an IgG1 Fc polypeptide sequence with knob and LALA mutations has the sequence of SEQ ID NO:104 or 100. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An IDUA-Fc fusion polypeptide comprising a mature, human IDUA sequence fused to the N-terminus of a TfR binding modified IgG1 Fc polypeptide sequence with knob and LALA mutations has the sequence of SEQ ID NO:101 or 102. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An Fc-IDUA fusion polypeptide comprising a mature human IDUA sequence fused to the C-terminus of a TfR binding modified IgG1 Fc polypeptide sequence with knob and LALA mutations has the sequence of SEQ ID NO:103. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An Fc-IDUA fusion polypeptide comprising a mature human IDUA sequence fused to the C-terminus of an IgG1 Fc polypeptide sequence with hole and LALAPS mutations has the sequence of SEQ ID NO:68. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

An Fc-IDUA fusion polypeptide comprising a mature human IDUA sequence fused to the C-terminus of an IgG1 Fc polypeptide sequence with hole and LALAPS mutations has the sequence of SEQ ID NO:69. The IDUA enzyme was joined to the Fc polypeptide by a GGGGS linker (SEQ ID NO:72) and the N-terminus of the Fc polypeptide included a portion of an IgG1 hinge region (DKTHTCPPCP; SEQ ID NO:6).

A TfR-binding modified Fc polypeptide with knob and LALA mutations has the sequence of SEQ ID NO:35 or 36. The N-terminus of the modified Fc polypeptide included a portion of an IgG1 hinge region (DKTHTCPPCP; SEQ ID NO:6).

A TfR-binding modified Fc polypeptide with knob and LALAPS mutations has the sequence of SEQ ID NO:37 or 38. The N-terminus of the modified Fc polypeptide included a portion of an IgG1 hinge region (DKTHTCPPCP; SEQ ID NO:6).

A first “N-terminal monozyme” IDUA-Fc fusion protein (“ETV:IDUA Fusion 1”) was generated, which comprises a TfR-binding modified Fc polypeptide having the sequence of SEQ ID NO:35 and an IDUA-Fc fusion polypeptide having the sequence of SEQ ID NO:50. The IDUA-Fc fusion protein may also be further processed during cell culture production, such that the TfR-binding modified Fc polypeptide has the sequence of SEQ ID NO:36 and/or the IDUA-Fc fusion polypeptide has the sequence of SEQ ID NO:51. Thus, as used herein, the term ETV:IDUA Fusion 1 may be used to refer to protein molecules having unprocessed sequences (i.e., SEQ ID NOs:35 and 50); protein molecules comprising one or more processed sequences (i.e., selected from SEQ ID NOs: 36 and 51); or to a mixture comprising processed and unprocessed protein molecules.

A second “N-terminal monozyme” IDUA-Fc fusion protein (“ETV:IDUA Fusion 2”) was generated, which comprises a TfR-binding modified Fc polypeptide having the sequence of SEQ ID NO:37 and an IDUA-Fc fusion polypeptide having the sequence of SEQ ID NO:58. The IDUA-Fc fusion protein may also be further processed during cell culture production, such that the TfR-binding modified Fc polypeptide has the sequence of SEQ ID NO:38 and/or the IDUA-Fc fusion polypeptide has the sequence of SEQ ID NO:59. Thus, as used herein, the term ETV:IDUA Fusion 2 may be used to refer to protein molecules having unprocessed sequences (i.e., SEQ ID NOs:37 and 58); protein molecules comprising one or more processed sequences (i.e., selected from SEQ ID NOs:38 and 59); or to a mixture comprising processed and unprocessed protein molecules.

A third “N-terminal monozyme” IDUA-Fc fusion protein (“ETV:IDUA Fusion 3”) was generated, which comprises a TfR-binding modified Fc polypeptide having the sequence of SEQ ID NO:37 and an IDUA-Fc fusion polypeptide having the sequence of SEQ ID NO:60. The IDUA-Fc fusion protein may also be further processed during cell culture production, such that the TfR-binding modified Fc polypeptide has the sequence of SEQ ID NO:38 and/or the IDUA-Fc fusion polypeptide has the sequence of SEQ ID NO:61. Thus, as used herein, the term ETV:IDUA Fusion 3 may be used to refer to protein molecules having unprocessed sequences (i.e., SEQ ID NOs:37 and 60); protein molecules comprising one or more processed sequences (i.e., selected from SEQ ID NOs:38 and 61); or to a mixture comprising processed and unprocessed protein molecules.

A fourth “N-terminal monozyme” IDUA-Fc fusion protein (“ETV:IDUA Fusion 4”) was generated, which comprises a TfR-binding modified Fc polypeptide having the sequence of SEQ ID NO:37 and an IDUA-Fc fusion polypeptide having the sequence of SEQ ID NO:64. The IDUA-Fc fusion protein may also be further processed during cell culture production, such that the TfR-binding modified Fc polypeptide has the sequence of SEQ ID NO:38 and/or the IDUA-Fc fusion polypeptide has the sequence of SEQ ID NO:65. Thus, as used herein, the term ETV:IDUA Fusion 4 may be used to refer to protein molecules having unprocessed sequences (i.e., SEQ ID NOs:37 and 64); protein molecules comprising one or more processed sequences (i.e., selected from SEQ ID NOs:38 and 65); or to a mixture comprising processed and unprocessed protein molecules.

A fifth “N-terminal monozyme” IDUA-Fc fusion protein (“ETV:IDUA Fusion 5”) was generated, which comprises a TfR-binding modified Fc polypeptide having the sequence of SEQ ID NO:37 and an IDUA-Fc fusion polypeptide having the sequence of SEQ ID NO:91. The IDUA-Fc fusion protein may also be further processed during cell culture production, such that the TfR-binding modified Fc polypeptide has the sequence of SEQ ID NO:38 and/or the IDUA-Fc fusion polypeptide has the sequence of SEQ ID NO:92. Thus, as used herein, the term ETV:IDUA Fusion 5 may be used to refer to protein molecules having unprocessed sequences (i.e., SEQ ID NOs:37 and 91); protein molecules comprising one or more processed sequences (i.e., selected from SEQ ID NOs:38 and 92); or to a mixture comprising processed and unprocessed protein molecules.

A “C-terminal monozyme” IDUA-Fc fusion protein (“ETV:IDUA Fusion 6”) was generated, which comprises a TfR-binding modified Fc polypeptide having the sequence of SEQ ID NO:37 and an IDUA-Fc fusion polypeptide having the sequence of SEQ ID NO:68. The IDUA-Fc fusion protein may also be further processed during cell culture production, such that the TfR-binding modified Fc polypeptide has the sequence of SEQ ID NO:38. Thus, as used herein, the term ETV:IDUA Fusion 6 may be used to refer to protein molecules comprising SEQ ID NOs:37 and 68; protein molecules comprising SEQ ID NOs: 38 and 68; or to a mixture having protein molecules comprising SEQ ID NOs: 37 and 68 and protein molecules comprising SEQ ID NOs:38 and 68.

Table 1 illustrates the sequences of additional exemplary IDUA-Fc fusion proteins and recombinant proteins.

TABLE 1
ETV:IDUA Fusion Proteins

| | Modified Fc | |
|---|---|---|
| | Polypeptide (optionally | IDUA-Fc or Fc-IDUA |
| Fusion Protein | linked to IDUA) | Fusion Polypeptide |
| ETV:IDUA Fusion 1 | SEQ ID NO: 35 or 36 | SEQ ID NO: 50 or 51 |
| ETV:IDUA Fusion 2 | SEQ ID NO: 37 or 38 | SEQ ID NO: 58 or 59 |
| ETV:IDUA Fusion 3 | SEQ ID NO: 37 or 38 | SEQ ID NO: 60 or 61 |
| ETV:IDUA Fusion 4 | SEQ ID NO: 37 or 38 | SEQ ID NO: 64 or 65 |
| ETV:IDUA Fusion 5 | SEQ ID NO: 37 or 38 | SEQ ID NO: 91 or 92 |
| ETV:IDUA Fusion 6 | SEQ ID NO: 37 or 38 | SEQ ID NO: 68 |
| ETV:IDUA Fusion 7 | SEQ ID NO: 37 or 38 | SEQ ID NO: 89 or 90 |
| ETV:IDUA Fusion 8 | SEQ ID NO: 35 or 36 | SEQ ID NO: 83 or 84 |
| ETV:IDUA Fusion 9 | SEQ ID NO: 35 or 36 | SEQ ID NO: 67 |
| ETV:IDUA Fusion 10 | SEQ ID NO: 101 or 102 | SEQ ID NO: 50 or 51 |
| ETV:IDUA Fusion 11 | SEQ ID NO: 103 | SEQ ID NO: 67 |

A composition comprising ETV:IDUA (e.g., any of the fusion proteins described above) may be used to refer to a composition comprising protein molecules having unprocessed sequences; protein molecules comprising one or more processed sequences; or to a mixture comprising processed and unprocessed protein molecules.

IDUA-Fc fusion proteins that lack the mutations that confer TfR binding were designed and constructed analogously.

A first non-TfR-binding “N-terminal monozyme” IDUA-Fc fusion protein (“IDUA-Fc Fusion 12” was generated, which comprises an Fc polypeptide having the sequence of SEQ ID NO:76 and an IDUA-Fc fusion polypeptide having the sequence of SEQ ID NO:83. The IDUA-Fc fusion protein may also be further processed during cell culture production, such that the Fc polypeptide has the sequence of SEQ ID NO:77 and/or the IDUA-Fc fusion polypeptide has the sequence of SEQ ID NO:84. Thus, as used herein, the term IDUA-Fc fusion protein may be used to refer to protein molecules having unprocessed sequences (i.e., SEQ ID NOs:76 and 83); protein molecules comprising one or more processed sequences (i.e., selected from SEQ ID NOs: 77 and 84); or to a mixture comprising processed and unprocessed protein molecules.

A second non-TfR-binding “N-terminal monozyme” IDUA-Fc fusion protein (“IDUA-Fc Fusion 13” was generated, which comprises an Fc polypeptide having the sequence of SEQ ID NO:76 and an IDUA-Fc fusion polypeptide having the sequence of SEQ ID NO:50. The IDUA-Fc fusion protein may also be further processed during cell culture production, such that the Fc polypeptide has the sequence of SEQ ID NO:77 and/or the IDUA-Fc fusion polypeptide has the sequence of SEQ ID NO:51. Thus, as used herein, the term IDUA-Fc fusion protein may be used to refer to protein molecules having unprocessed sequences (i.e., SEQ ID NOs:76 and 50); protein molecules comprising one or more processed sequences (i.e., selected from SEQ ID NOs: 77 and 51); or to a mixture comprising processed and unprocessed protein molecules.

A non-TfR-binding “N-terminal bizyme” IDUA-Fc fusion protein (“IDUA-Fc Fusion 14” was generated, which comprises a first IDUA-Fc polypeptide having the sequence of SEQ ID NO:104 and a second IDUA-Fc fusion polypeptide having the sequence of SEQ ID NO:50. The IDUA-Fc fusion protein may also be further processed during cell culture production, such that the first IDUA-Fc polypeptide has the sequence of SEQ ID NO:100 and/or the second IDUA-Fc fusion polypeptide has the sequence of SEQ ID NO:51. Thus, as used herein, the term IDUA-Fc Fusion 14 may be used to refer to protein molecules having unprocessed sequences (i.e., SEQ ID NOs:104 and 50); protein molecules comprising one or more processed sequences (i.e., selected from SEQ ID NOs:100 and 51); or to a mixture comprising processed and unprocessed protein molecules. Table 2 illustrates the sequences of exemplary non-TfR binding IDUA-Fc fusion proteins and recombinant proteins.

TABLE 2
IDUA-Fc Fusion Proteins and Recombinant Proteins

| | Fc Polypeptide | IDUA-Fc or Fc- | |
|---|---|---|---|
| | (optionally | IDUA Fusion | IDUA |
| Fusion Protein | linked to IDUA) | Polypeptide | Polypeptide |
| IDUA-Fc Fusion 12 | SEQ ID NO: 76 | SEQ ID NO: 83 | — |
| | or 77 | or 84 | |
| IDUA-Fc Fusion 13 | SEQ ID NO: 76 | SEQ ID NO: 50 | — |
| | or 77 | or 51 | |
| IDUA-Fc Fusion 14 | SEQ ID NO: 104 | SEQ ID NO: 50 | — |
| | or 100 | or 51 | |
| IDUA Recombinant | — | — | SEQ ID |
| Protein 15 | | | NO: 93 |
| IDUA Recombinant | — | — | SEQ ID |
| Protein 16 | | | NO: 94 |
| IDUA Recombinant | — | — | SEQ ID |
| Protein 17 | | | NO: 95 |

A composition comprising ETV:IDUA (e.g., any of the fusion proteins described above) may be used to refer to a composition comprising protein molecules having unprocessed sequences; protein molecules comprising one or more processed sequences; or to a mixture comprising processed and unprocessed protein molecules.

### Recombinant Protein Expression and Purification

To express recombinant IDUA-Fc fusion proteins, ExpiCHO cells (Thermo Fisher Scientific) were transfected with relevant DNA constructs using Expifectamine™ CHO transfection kit according to manufacturer's instructions (Thermo Fisher Scientific). Cells were grown in ExpiCHO™ Expression Medium supplemented with feed as described by the manufacturer's protocol at 37° C., 5% CO₂ and 125 rpm in an orbital shaker (Infors HT Multitron). In brief, logarithmic growing ExpiCHO cells were transfected at 6×10⁶ cells/ml density with 0.8 μg of total DNA plasmid per mL of culture volume. After transfection, cells were returned to 37° C. 18-22 hours post transfection the transfected cultures were supplemented with a nutrient feed and the cell culture temperature was reduced to 32° C. for the duration of the production run. Transfected cell culture supernatants were harvested 120 hours post transfection by centrifugation at 4000 rpm for 15 mins. Clarified supernatants were filtered (0.22 μM membrane) and stored at 4° C.

IDUA-Fc fusion proteins with (or without) engineered Fc regions conferring TfR binding were purified from cell culture supernatants using Protein A affinity chromatography. Supernatants were loaded onto a HiTrap MabSelect Prisma Protein A affinity column (GE Healthcare Life Sciences using an Akta Pure System). The column was then washed with 10 column volumes (CVs) of PBS. Bound proteins were eluted using 50 mM sodium citrate buffer pH 3.6. Immediately after elution, fractions were neutralized using 1 M Tris pH8 (at a 1:8 dilution). The Protein A pools underwent further polishing using cation exchange chromatography (CEX). The Protein A pool was diluted 10 fold with sodium acetate buffer, pH 5.5 to facilitate binding on a HiTrap® SP High Performance column (Cytiva, SKU 17-1152-01). The column was eluted with 20 mM sodium acetate, 0.5M NaCl, pH 5.5 using linear salt gradient for 30CV. The fractions were further analyzed by HPLC-SEC. Fractions with purity >95% were pooled and dialyzed in 1×PBS, pH 7.4. Homogeneity of IDUA-Fc fusions final bulks was assessed by a number of techniques including reducing and non-reducing Caliper (microcapillary electrophoresis-SDS) and HPLC-SEC.

Recombinant IDUA with C-terminal hexahistidine tags (SEQ ID NOs:93-95) (“Hexahistidine” is disclosed as SEQ ID NO: 96) were expressed in ExpiCHO cells as described above. To purify the hexahistadine-tagged IDUA enzymes (“Hexahistidine” is disclosed as SEQ ID NO: 96), transfected supernatants were dialyzed against 15 L of 20 mM HEPES pH 7.4 containing 100 mM NaCl overnight. Dialyzed supernatants were bound to a HisTrap column (GE Healthcare Life Sciences using an Akta Pure System). After binding, the column was washed with 20 CV of PBS. Bound proteins were eluted using PBS containing 500 mM imidazole. Pooled fractions containing IDUA enzyme were diluted 1:10 in 50 mM Tris pH 7.5 and further purified using Q Sepharose High Performance (GE Healthcare). After binding, the column was washed with 10 CV of 50 mM Tris pH 7.5. Bound proteins were eluted using a linear gradient to 50 mM Tris pH 7.5 and 0.5 M NaCl and collected in 1 CV fractions. Fraction purity was assessed by non-reducing SDS-PAGE.

### Example 2: Characterization of IDUA-Fc Fusion Proteins

ETV:IDUA fusion proteins constructed and prepared as described in Example 1 were evaluated for TfR binding, enzymatic activity, and cellular potency.

IDUA-Fc Fusion Proteins with Engineered TfR Binding Site Bind to Human TfR

To determine whether IDUA-Fc fusion proteins with engineered TfR binding affects the ability of the modified Fc domain to interact with human TfR, the affinity of ETV:IDUA Fusions 3, 4, and 6 (Example 1) for human TfR was measured by surface plasmon resonance (SPR) using a Biacore 8K instrument (Cytiva). Five (5) μg/mL of the IDUA-Fc fusion proteins were captured for 1 minute on Protein A-coated Biacore™ Series S CM5 sensor chip and serial 3-fold dilutions of human apical domain TfR were injected at a flow rate of 30 μL/min. Each sample was analyzed with five consecutive 60-second injections with increasing human TfR concentrations followed by a 60-second dissociation at the end of the injection cycle. After each injection, the chip was regenerated using 10 mM glycine-HCl (pH 1.5). Binding response was corrected by subtracting the RU from a flow cell capturing an irrelevant IgG at similar density A. 1:1 Languir model of simultaneous fitting of kₒₙ and k_{off} was used for kinetics analysis using Biacore™ Insight Evaluation Software. The monovalent binding affinities of ETV:IDUA Fusions 3, 4, and 6 for human TfR ranged from about 200 nM to 400 nM.

IDUA-Fc Fusion Proteins with Engineered TfR Binding Site are Active In Vitro

The in vitro activity of engineered TfR-binding IDUA-Fc fusion proteins were assessed to demonstrate that IDUA maintains its enzymatic activity when fused to the human IgG fragment. The in vitro activity of recombinant IDUA was measured using a one-step fluorometric enzymatic assay using an artificial substrate. An aliquot of 70.96 mM 4-Methylumbelliferyl-α-L-Iduronide (free acid) (Cayman Chemical #19543) was diluted in Assay Buffer (50 mM sodium acetate, 0.5 M NaCl, 0.025% Triton X-100, pH 4.5) to a final concentration of 2.5 mM. IDUA-Fc fusion proteins were serially diluted starting from a concentration of 50 mM. Five (5) μL substrate was then mixed with 5 μL of the serially diluted IDUA-Fc fusion protein in a 384-well black, flat bottom microplate plate (NUNC #262260). The reaction was incubated for 60 minutes at 22° C. and terminated with 10 μL of Stop Buffer (0.5 M sodium carbonate buffer, pH 10.3). Fluorescence of the reaction solution was then measured (excitation at 365 nm and emission at 450 nm). A 4-Methylumbelliferone standard curve was fit by non-linear regression to calculate the amount of product and verified as less than 10% of total substrate cleavage. Specific activity (pmol product/min/pmol IDUA) was calculated by dividing the amount of product by the reaction time and molar amount of IDUA.

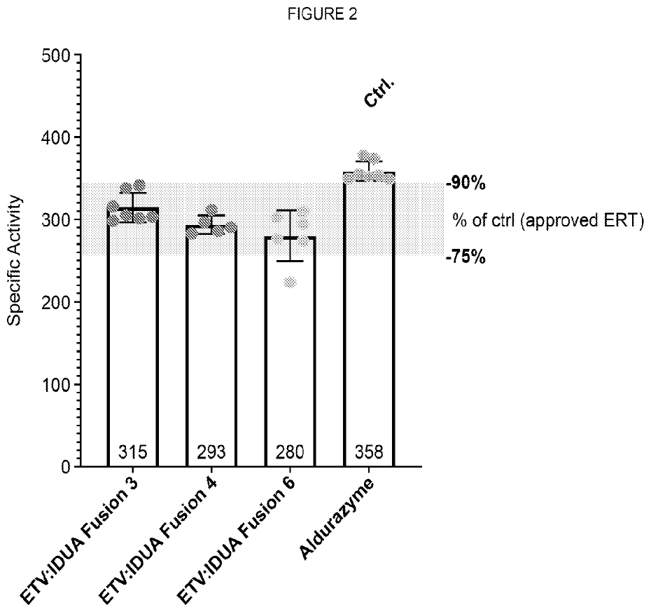

The in vitro enzymatic activity assay demonstrated that IDUA-Fc fusion proteins were active and were similar between ETV:IDUA Fusions 3, 4, and 6 (FIG. 2). All ETV:IDUA fusion proteins had specific activity that ranged from about 75% to about 90% the activity of Aldurazyme (laronidase).

IDUA-Fc Fusion Proteins with Engineered TfR Binding Site Corrects Substrate Accumulation In Vitro

The cellular activity of IDUA-Fc fusion proteins was also examined in fibroblasts from Hurler (MPS I) patients and healthy controls using a LCMS quantification of heparan sulfate and dermatan sulfate to assess substrate correction. Briefly, Hurler fibroblasts and healthy fibroblasts were plated in 24-well culture plates in DMEM media supplemented with 10% FBS at a density of 75,000 cells per well. Cells were cultured in this format until they reached confluency, approximately 96 hours. ETV:IDUA Fusion 1 was then added to each well in a 12-point, 5-fold serially diluted dose curve from a starting concentration of 62.5 nM. Seventy-two (72) hours after addition of protein, cells were lysed by hypotonic shock. Lysates were transferred to 96-well assay plates, buffer composition was adjusted to contain 111 mM ammonium acetate and 11 mM calcium acetate, followed by sonication to complete lysis. Protein concentration of lysates was determined via BCA assay, and all lysates were adjusted to equal protein concentration. Lysates were supplemented with 2 mM DTT and GAGs were digested with an enzymatic mixture containing Heparinase I (1.25 mIU/reaction), Heparinase II (1.25 mIU/reaction), Heparinase III (1.25 mIU/reaction), and Chondroitinase B (6.25 mIU/reaction) for 3 hours at 30° C. The digestion reaction was quenched with 10 mM EDTA, and 20 ng of 4UA-2S-GlcNCOEt-6S internal standard was added to each reaction, followed by denaturation at for 10 minutes at 95° C. Insoluble material was removed from the samples by centrifugal filtration, and clarified samples were mixed 1:1 with acetonitrile in glass LC-MS vials. Disaccharide species derived from heparan and dermatan sulfate GAGs were quantified by LC-MS/MS on a Xevo TQ-S Micro instrument equipped with a ACQUITY UPLC BEH Amide 1.7 mm, 2.1×150 mm column.

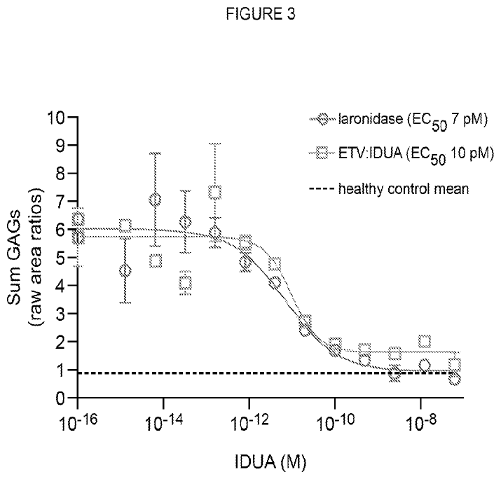

MPS I patient fibroblasts lack IDUA activity, leading to an accumulation of heparan sulfate and dermatan sulfate, two glycosaminoglycan (GAG) species. ETV:IDUA Fusion 1 showed similar potency to laronidase in MPS I patient-derived cells, displaying a low picomolar cellular EC₅₀ (about 7-10 pM) for reducing the accumulation of heparan sulfate (FIG. 3).

IDUA-Fc Fusion Proteins with Engineered TfR Binding Site Show Increased Brain Uptake in TfR KI Mice

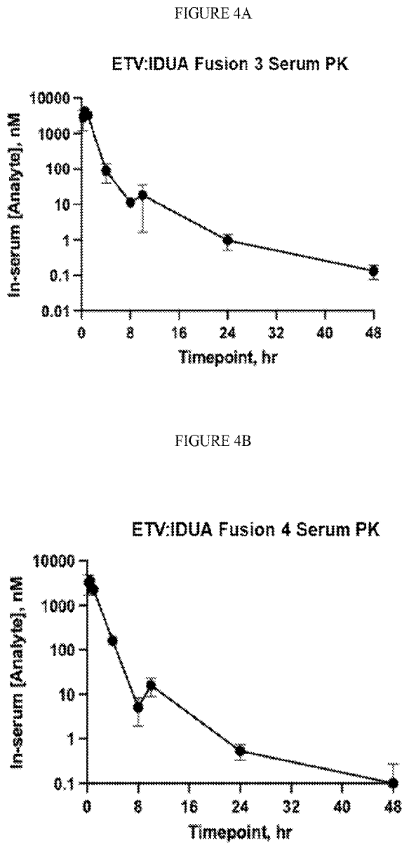

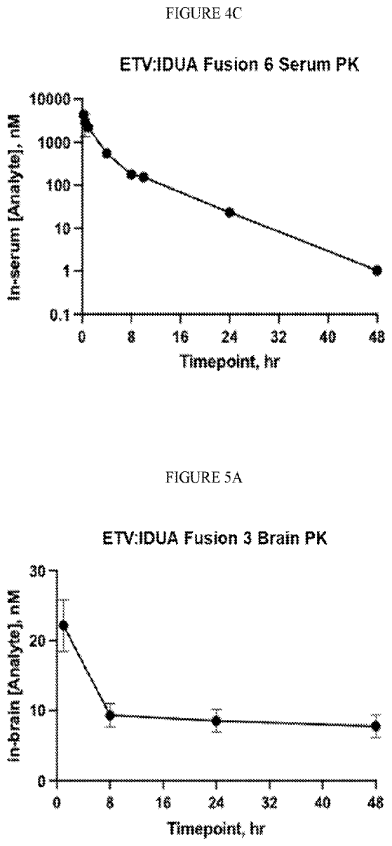

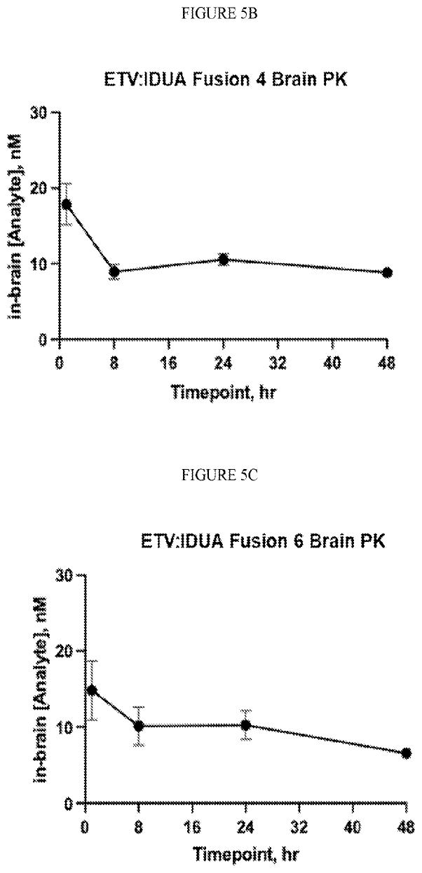

The peripheral (serum) and brain PK of IDUA-Fc fusion proteins in TfR knock in (referenced herein as “TfR^{mu/hu}KI” or “TfR^{mu/hu}”) mice were evaluated. TfR^{mu/hu}KI mice were generated as described in International Patent Publication No. WO 2018/152285 using CRISPR/Cas9 technology to express human Tfrc apical domain within the murine Tfrc gene; the resulting chimeric TfR was expressed in vivo under the control of the endogenous promoter. Briefly, 6-8 weeks old male TfR^{mu/hu}KI mice (n=12 per cohort) were dosed with 40 mg/kg of ETV:IDUA fusion protein, and the concentration of IDUA enzyme and ETV:IDUA intact molecule was measured in serum and brain tissue. Total IDUA enzyme levels were measured using a sandwich ELISA-based assay at t=0.25, 0.5, 1, 4, 8, 10, 24, and 48 hours post-dose for serum, and at t=1, 8, 24, 48 hours post-dose for Brain PK. The IDUA-Fc fusion proteins that were used in the analysis are described above and were prepared in accordance with Example 1. For measurement of total IDUA enzyme levels, a polyclonal sheep anti-human IDUA antibody (Bio-Techne AF4119) was coated onto a MULTI-ARRAY® 96-well plate (Meso Scale Diagnostics L15XA-3) overnight. The plate was blocked with Blocker™ Casein in PBS (ThermoFisher 37528), BS and then incubated with diluted serum or brain lysate. Next, a Ruthenium-conjugated polyclonal sheep anti-human IDUA antibody (Bio-Techne AF4119) was added for detection. 1× Read buffer T with surfactant (Meso Scale Diagnostics R92TC-1) was added to each well, and plated were loaded to MSD reader. The standard curves were based on the individual constructs and were fit using a four-parameter logistic curve. The results are illustrated in FIGS. 4A-4C and 5A-5C as well as Table 3.

ETV:IDUA Fusions 3, 4, and 6 showed good stability in circulation. High brain Cmax values were observed for all molecules (about 15 nM or higher), and brain uptake of all molecules was consistent with peripheral exposure.

TABLE 3
Pharmacokinetic parameters of ETV:IDUA
fusion proteins (TfR^{mu/hu}KI mice)

| | CL | Serum half- | Serum | Brain |
|---|---|---|---|---|
| Molecule | (mL/hr/kg) | life (hr) | Cmax (nM) | Cmax (nM) |
| ETV:IDUA | 37.0 | 5.8 | 4242 | 22.16 |
| Fusion 3 | | | | |
| ETV:IDUA | 45.3 | 6.0 | 3526 | 17.87 |
| Fusion 4 | | | | |
| ETV:IDUA | 30.1 | 5.3 | 4308 | 14.83 |
| Fusion 6 | | | | |

### Example 3: Product Quality Attributes of ETV:IDUA Fusions

Various ETV:IDUA fusion proteins were evaluated in terms of product quality. For this study, ETV:IDUA Fusions 3, 4, and 6 (Example 1) were assessed. All structures were prepared as described in Example 1. Homogeneity of ETV:IDUA fusion proteins in the eluted fractions was assessed by a number of techniques including reducing and non-reducing Caliper (microcapillary electrophoresis-SDS) and HPLC-SEC. Affinity for human TfR was measured as described in Example 2.

### Results

As described in Example 2, the measured human TfR affinities for ETV:IDUA Fusions 3 and 6 were comparable (K_{D} ranging from about 200 to 400 nM).

The expression titer for ETV:IDUA Fusions 3, 4, and 6 were greater than about 100 mg/L.

Post protein A chromatography purification recovery of ETV:IDUA Fusions 3, 4, and 6 was evaluated. Analysis of post-protein A pools of all structures illustrated at least 85% purity (as measured by HPLC-SEC) with intact ETV structure (maintenance of modified Fc dimer comprising knob and hole pair) of at least about 90%. The post-protein A pools of all structures underwent cation exchange chromatography (CEX) for further polishing. Post-CEX pools of all structures achieved purity levels of >99% (as measured by HPLC-SEC) with intact ETV structure of >95%.

A summary of the product quality attributes of the ETV:IDUA fusion proteins is provided in Table 4.

TABLE 4
Product quality attributes of ETV:IDUA fusion proteins

| | ETV:IDUA | ETV:IDUA | ETV:IDUA |
|---|---|---|---|
| Attribute | Fusion 3 | Fusion 4 | Fusion 6 |
| Expression titer (mg/L) | 182 | 160 | 105 |
| % yield (post-Protein A) | ~45-50 | ~45-50 | 70 |
| % purity (post-Protein A) | 93 | 93 | 85 |
| % purity (post-CEX) | 99 | 99 | 99 |
| Specific Activity | 315 [89%] | 293 [83%] | 280 [79%] |
| (pmol prod/min/pmol IDUA) | | | |
| [% relative to laronidase] | | | |
| TfR affinity K_{D} | 340 nM | 355 nM | 200 nM |

### Example 4: A Comparative Study of ETV:IDUA Fusion Protein with Laronidase in a Disease Model of MPS I

An exemplary ETV:IDUA fusion protein (ETV:IDUA Fusion 3) was compared to a standard-of-care enzyme replacement therapy, laronidase, in a mouse model of MPS I.

### Results

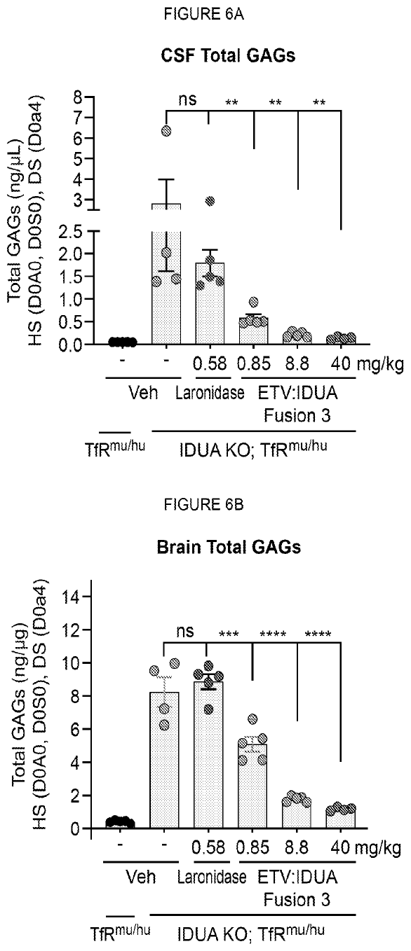

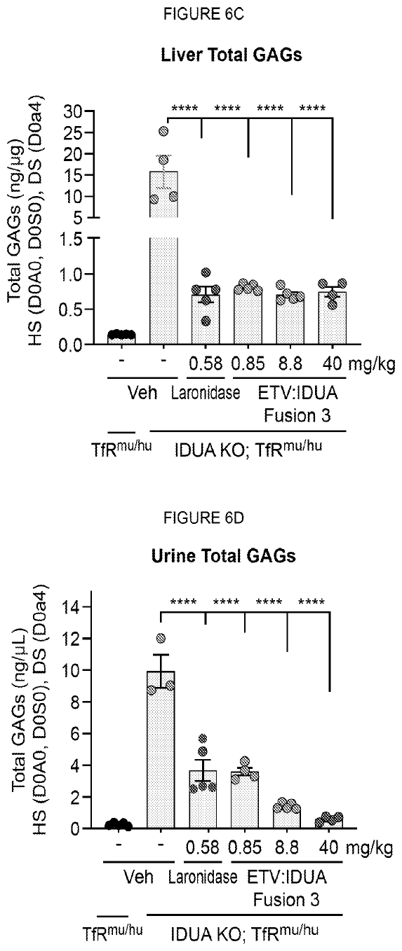

ETV:IDUA and laronidase were compared in how well the proteins reduced total GAG levels in the brain, CSF, and liver of IDUA KO;TfR^{mu/hu} KI mice after a single intravenous (IV) dose. ETV:IDUA Fusion 3 was administered to the mice as an equimolar dose to that of laronidase (0.85 mg/kg), at a dose about 10-fold greater than equimolar dose (8.8 mg/kg), and at a high dose level (40 mg/kg), while laronidase was administered at a dose consistent with a clinically relevant dose for treatment (0.58 mg/kg). Total GAGs were determined as the sum of the major heparan sulfate (HS) (D0A0, D0S0) and dermatan sulfate (DS) (D0a4)-derived disaccharides. As illustrated in FIGS. 6A-6D and Table 5, at 7 days following a single dose, ETV:IDUA protein was able to reduce total GAG levels in both the cerebrospinal fluid (CSF) and brain tissue, and this reduction was better than the effect achieved with laronidase. The reduction in total GAG levels was seen at all three administered doses of ETV:IDUA, including the dose that was equimolar to the dose of laronidase (CSF: 79% reduction vs. 36% reduction; brain: 38% reduction vs. N/S reduction). In the liver, total GAG reduction was similar between laronidase and ETV:IDUA protein across all doses administered. In urine, ETV:IDUA was comparable at reducing total GAG levels at the equimolar dose to that of laronidase (0.85 mg/kg). However, the reduction of total GAG levels in urine was better than that of laronidase at higher doses of ETV:IDUA (e.g., at 8.8 mg/kg and 40 mg/kg).

TABLE 5
Percent reduction in Total GAG levels after single IV dose administration
compared to vehicle-treated IDUA KO; TfR^{mu/hu} KI mice.

| | Dose | CSF Total | Brain Total | Liver Total | Urine Total |
|---|---|---|---|---|---|
| Treatment | [mg/kg] | GAG reduction | GAG reduction | GAG reduction | GAG reduction |
| Laronidase | 0.58 | 36% | N/S | 96% | 63% |
| ETV:IDUA Fusion 3 | 0.85 | 79% | 38% | 95% | 64% |
| ETV:IDUA Fusion 3 | 8.8 | 92% | 79% | 96% | 86% |
| ETV:IDUA Fusion 3 | 40 | 95% | 86% | 95% | 94% |

The data in FIGS. 6A-6D represent approximate mean amounts+/−standard error of the mean. The results demonstrate that ETV:IDUA robustly reduced substrate accumulation in the CSF, brain, and urine and this reduction represented an improvement relative to a standard-of-care ERT treatment.

### Experimental Methods

ETV:IDUA Fusion 3 was expressed and purified as described in Example 1. Laronidase was obtained from a commercial source (NDC 58468-0070-1, Lot number 0Y0432).

The MPS I mouse model used in this study is a mouse model in which the gene encoding IDUA is knocked-out while also harboring the human TfR apical domain knocked into the murine TfR (referred to herein as “IDUA KO; TfR^{mu/hu} KI” or “IDUA KO; TfR^{mu/hu}” mice). IDUA KO mice were obtained from The Jackson Laboratories (JAX stock #004068). Briefly, TfR^{mu/hu} KI male mice (see, Example 2; also referred herein as “TfR^{mu/hu}”) were bred to female mice heterozygous for the IDUA mutation to generate homozygous IDUA KO mice in a TfR^{mu/hu} KI homozygous background. Mice used in this study were mixed sex and housed under a 12 hour light-dark cycle with ad libitum access to food (#25502, irradiated; LabDiet) and water.

In the study, IDUA KO;TfR^{mu/hu} KI mice were administered a single dose of ETV:IDUA Fusion 3 or laronidase via intravenous injection, and pharmacodynamic responses were assessed. In particular, the effect of peripheral administration of the proteins on brain, CSF, liver, and urine total GAG levels of IDUA KO;TfR^{mu/hu} KI mice was determined. Approximately 2-month-old IDUA KO;TfR^{mu/hu} KI were injected intravenously (i.v.) with saline, ETV:IDUA fusion protein (0.85, 8.8, or 40 mg/kg body weight), or laronidase (0.58 mg/kg body weight) (n=4-5/group). Approximately 2-month-old littermate TfR^{mu/hu} KI mice (non-MPS I mice) injected i.v. with saline were used as controls. Urine samples were collected at six (6) days post-single dose as well as at seven (7) days post-single dose and pooled for analysis. At seven (7) days post-single dose, all animals were sacrificed, and brain, CSF, and liver tissue were collected. All collected tissues and fluids were flash-frozen on dry ice and stored at −80° C. until analysis.

Heparan sulfate and dermatan sulfate species were measured in vivo using LC-MS/MS-based methods as described below. Tissue aliquots (50 mg) were homogenized in water (500-750 L) using the Qiagen TissueLyzer II for 3 minutes at 30 Hz twice. Homogenate was transferred to a 96-well deep plate and sonicated using a 96-tip sonicator (Q Sonica) for 20×1 second pulses. Sonicated homogenates were spun at 17,000×g for 20 minutes at 4° C. to pellet cell debris. The resulting lysate was transferred to a clean 96-well deep plate, and a BCA was performed to quantify total protein. Heparan sulfate (HS) and dermatan sulfate (DS) in the samples were digested to their corresponding disaccharides prior to LC-MS/MS analysis. Protein lysates (from brain and liver tissues) and CSF and urine were mixed with Heparinases I, II, and III and internal standard D4UA-2S-GlcNCOEt-6S (HD009, Iduron Ltd, Manchester, UK) in digestion buffer consisting of ammonium acetate, calcium acetate, and DTT for 3 hours with shaking (700 rpm) at 30° C. in a PCR plate. For urine, stable-labeled creatinine (internal standard, N-METHYL-D3, 98%) was also added into the mix. After 3 hours of incubation, 0.0025 M of EDTA was added to each sample to stop the reaction and the mixture was boiled at 95° C. for 10 minutes to inactivate the enzymes. The digested samples from brain, liver, CSF, and urine were centrifuged, and supernatants were transferred to a cellulose acetate filter plate (Millipore, MSUN03010) and centrifuged again. For brain, CSF, and urine samples, the resulting eluent was mixed with equal parts of acetonitrile in 96-well glass vial plates and analyzed by mass spectrometry as described below. For liver the resulting eluent was mixed with 2× the volume of acetonitrile.

Quantification of HS and DS derived disaccharides in fluids and tissues were performed by liquid chromatography (Exion LC, Sciex, Framingham, MA, USA; Agilent 1290 Infinity II HPLC, Agilent Technologies Inc., Santa Clara, CA, USA) coupled to electrospray mass spectrometry (Sciex Triple Quad 7500, Sciex, Framingham, MA, USA; Sciex Triple Quad 6500+, Sciex, Framingham, MA, USA). For each analysis on both liquid chromatography systems, sample was injected on a ACQUITY UPLC BEH Amide 1.7 mm, 2.1×150 mm column (Waters Corporation, Milford, MA, USA) using a flow rate of 0.6 mL/minute with a column temperature of 55° C. Mobile phase A consisted of water with 10 mM ammonium formate and 0.1% formic acid, and mobile phase B consisted of acetonitrile with 0.1% formic acid. The gradient was programmed as follows: 0.0-6.0 minutes at 80% B, 6.0-6.01 minutes from 80% B to 20% B, 6.01-8.0 minutes 20% B to 20% B, 8.0-8.1 minutes 20% B to 80% B and 8.1-10 minutes hold at 80% B. For Sciex Triple Quad 7500, electrospray ionization was performed in negative-ionization mode applying the following settings: curtain gas at 40; ion spray voltage at −4500; temperature at 450° C.; ion source Gas 1 at 50; and ion source Gas 2 at 60. Data acquisition was performed using Sciex OS 2.1.6.59781 in multiple reaction monitoring mode (MRM) with the following settings: dwell time at 100 msec; collision energy at −30; entrance potential at −10; collision cell exit potential at −10. For Sciex Triple Quad 6500+, electrospray ionization was performed in negative-ionization mode applying the following settings: curtain gas at 20; ion spray voltage at −4500; temperature at 450° C.; ion source Gas 1 at 50; and ion source Gas 2 at 60. Data acquisition was performed using Analyst 1.7.1. in MRM with the following settings: dwell time at 100 msec; collision energy at −30; declustering potential at −80; entrance potential at −10; collision cell exit potential at −10. Individual disaccharide species were identified based on their retention times and MRM transitions using commercially available reference standards (Iduron Ltd). The following disaccharide transitions were monitored for Sciex Triple Quad 7500 and Sciex Triple Quad 6500+: D0A0 (HS), m/z 378>87; D0S0 (HS), m/z 416>138, and D0a4 at m/z 458>300; D4UA-2S-GlcNCOEt-6S (internal standard) m/z 472>97. Disaccharide amounts were normalized to total protein levels as measured by a BCA assay, or to the volume of body fluid used per sample.

Informal Sequence Listing

| SEQ ID | | |
|---|---|---|
| NO: | Sequence | Description |
| 1 | APELLGGPSVFLFPPKPKDTLMISRTPEVTCVVVDVSHEDPEVKFNWYVDGV | Wild-type human |
| | EVHNAKTKPREEQYNSTYRVVSVLTVLHQDWLNGKEYKCKVSNKALPAPIEK | Fc sequence |
| | TISKAKGQPREPQVYTLPPSRDELTKNQVSLTCLVKGFYPSDIAVEWESNGQ | positions 231- |
| | PENNYKTTPPVLDSDGSFFLYSKLTVDKSRWQQGNVFSCSVMHEALHNHYTQ | 447 EU index |
| | KSLSLSPGK | numbering |
| 2 | APELLGGPSVFLFPPKPKDTLMISRTPEVTCVVVDVSHEDPEVKFNWYVDGV | Wild-type human |
| | EVHNAKTKPREEQYNSTYRVVSVLTVLHQDWLNGKEYKCKVSNKALPAPIEK | Fc sequence |
| | TISKAKGQPREPQVYTLPPSRDELTKNQVSLTCLVKGFYPSDIAVEWESNGQ | positions 231- |
| | PENNYKTTPPVLDSDGSFFLYSKLTVDKSRWQQGNVFSCSVMHEALHNHYTQ | 446 EU index |
| | KSLSLSPG | numbering |
| 3 | APELLGGPSVFLFPPKPKDTLMISRTPEVTCVVVDVSHEDPEVKFNWYVDGV | CH2 domain |
| | EVHNAKTKPREEQYNSTYRVVSVLTVLHQDWLNGKEYKCKVSNKALPAPIEK | sequence |
| | TISKAK | positions 231- |
| | | 340 EU index |
| | | numbering |
| 4 | GQPREPQVYTLPPSRDELTKNQVSLTCLVKGFYPSDIAVEWESNGQPENNYK | CH3 domain |
| | TTPPVLDSDGSFFLYSKLTVDKSRWQQGNVFSCSVMHEALHNHYTQKSLSLS | sequence |
| | PGK | Positions 341- |
| | | 447 EU index |
| | | numbering |
| 5 | EPKSCDKTHTCPPCP | Human IgG1 hinge |
| | | amino acid |
| | | sequence |
| 6 | DKTHTCPPCP | Portion of human |
| | | IgG1 hinge |
| | | sequence |
| 7 | MMDQARSAFSNLFGGEPLSYTRFSLARQVDGDNSHVEMKLAVDEEENADNNT | Human |
| | KANVTKPKRCSGSICYGTIAVIVFFLIGFMIGYLGYCKGVEPKTECERLAGT | transferrin |
| | ESPVREEPGEDFPAARRLYWDDLKRKLSEKLDSTDFTGTIKLLNENSYVPRE | receptor protein |
| | AGSQKDENLALYVENQFREFKLSKVWRDQHFVKIQVKDSAQNSVIIVDKNGR | 1 (TFR1) |
| | LVYLVENPGGYVAYSKAATVTGKLVHANFGTKKDFEDLYTPVNGSIVIVRAG | |
| | KITFAEKVANAESLNAIGVLIYMDQTKFPIVNAELSFFGHAHLGTGDPYTPG | |
| | FPSFNHTQFPPSRSSGLPNIPVQTISRAAAEKLFGNMEGDCPSDWKTDSTCR | |
| | MVTSESKNVKLTVSNVLKEIKILNIFGVIKGFVEPDHYVVVGAQRDAWGPGA | |
| | AKSGVGTALLLKLAQMFSDMVLKDGFQPSRSIIFASWSAGDFGSVGATEWLE | |
| | GYLSSLHLKAFTYINLDKAVLGTSNFKVSASPLLYTLIEKTMQNVKHPVTGQ | |
| | FLYQDSNWASKVEKLTLDNAAFPFLAYSGIPAVSFCFCEDTDYPYLGTTMDT | |
| | YKELIERIPELNKVARAAAEVAGQFVIKLTHDVELNLDYERYNSQLLSFVRD | |
| | LNQYRADIKEMGLSLQWLYSARGDFFRATSRLTTDFGNAEKTDRFVMKKLND | |
| | RVMRVEYHFLSPYVSPKESPFRHVFWGSGSHTLPALLENLKLRKQNNGAFNE | |
| | TLFRNQLALATWTIQGAANALSGDVWDIDNEF | |
| 8 | NSVIIVDKNGRLVYLVENPGGYVAYSKAATVTGKLVHANFGTKKDFEDLYTP | Human TER apical |
| | VNGSIVIVRAGKITFAEKVANAESLNAIGVLIYMDQTKFPIVNAELSFFGHA | domain |
| | HLGTGDPYTPGFPSFNHTQFPPSRSSGLPNIPVQTISRAAAEKLFGNMEGDC | |
| | PSDWKTDSTCRMVTSESKNVKLTVS | |
| 9 | APELLGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | hole mutations |
| | TISKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHYTQ | |
| | KSLSLSPGK | |
| 10 | APELLGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | hole mutations |
| | TISKAKGQPREPQVYTLPPSRDELTKNQVSLSCAVKGFYPSDIAVEWESNGQ | |
| | PENNYKTTPPVLDSDGSFFLVSKLTVDKSRWQQGNVFSCSVMHEALHNHYTQ | |
| | KSLSLSPG | |
| 11 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | hole and LALA |
| | TISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQ | |
| | KSLSLSPGK | |
| 12 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | hole and LALA |
| | TISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQ | |
| | KSLSLSPG | |
| 13 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALGAPIEK | hole and LALAPG |
| | TISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQ | |
| | KSLSLSPGK | |
| 14 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALGAPIEK | hole and LALAPG |
| | TISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQ | |
| | KSLSLSPG | |
| 15 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALSAPIEK | hole and LALAPS |
| | TISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQ | |
| | KSLSLSPGK | |
| 16 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALSAPIEK | hole and LALAPS |
| | TISKAKGQPREPQVYTLPPSRDELTKNQVSLSCAVKGFYPSDIAVEWESNGQ | mutations |
| | PENNYKTTPPVLDSDGSFFLVSKLTVDKSRWQQGNVFSCSVMHEALHNHYTQ | |
| | KSLSLSPG | |
| 17 | APELLGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | knob mutation |
| | TISKAKGQPREPQVYTLPPSRDELTKNQVSLWCLVKGFYPSDIAVEWESNGQ | |
| | PENNYKTTPPVLDSDGSFFLYSKLTVDKSRWQQGNVFSCSVMHEALHNHYTQ | |
| | KSLSLSPGK | |
| 18 | APELLGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | knob mutation |
| | TISKAKGQPREPQVYTLPPSRDELTKNQVSLWCLVKGFYPSDIAVEWESNGQ | |
| | PENNYKTTPPVLDSDGSFFLYSKLTVDKSRWQQGNVFSCSVMHEALHNHYTQ | |
| | KSLSLSPG | |
| 19 | DKTHTCPPCPAPEAAGGPSVFLFPPKPKDTLMISRTPEVTCVVVDVSHEDPE | Fc sequence with |
| | VKFNWYVDGVEVHNAKTKPREEQYNSTYRVVSVLTVLHQDWLNGKEYKCKVS | hole and LALA |
| | NKALPAPIEKTISKAKGQPREPQVYTLPPSRDELTKNQVSLSCAVKGFYPSD | mutations and |
| | IAVEWESNGQPENNYKTTPPVLDSDGSFFLVSKLTVDKSRWQQGNVFSCSVM | portion of human |
| | HEALHNHYTQKSLSLSPGK | IgG1 hinge |
| | | sequence |
| 20 | DKTHTCPPCPAPEAAGGPSVFLFPPKPKDTLMISRTPEVTCVVVDVSHEDPE | Fc sequence with |
| | VKFNWYVDGVEVHNAKTKPREEQYNSTYRVVSVLTVLHQDWLNGKEYKCKVS | hole and LALA |
| | NKALPAPIEKTISKAKGQPREPQVYTLPPSRDELTKNQVSLSCAVKGFYPSD | mutations and |
| | IAVEWESNGQPENNYKTTPPVLDSDGSFFLVSKLTVDKSRWQQGNVFSCSVM | portion of human |
| | HEALHNHYTQKSLSLSPG | IgG1 hinge |
| | | sequence |
| 21 | DKTHTCPPCPAPEAAGGPSVFLFPPKPKDTLMISRTPEVTCVVVDVSHEDPE | Fc sequence with |
| | VKFNWYVDGVEVHNAKTKPREEQYNSTYRVVSVLTVLHQDWLNGKEYKCKVS | hole and LALAPS |
| | NKALSAPIEKTISKAKGQPREPQVYTLPPSRDELTKNQVSLSCAVKGFYPSD | mutations and |
| | IAVEWESNGQPENNYKTTPPVLDSDGSFFLVSKLTVDKSRWQQGNVFSCSVM | portion of human |
| | HEALHNHYTQKSLSLSPGK | IgG1 hinge |
| | | sequence |
| 22 | DKTHTCPPCPAPEAAGGPSVFLFPPKPKDTLMISRTPEVTCVVVDVSHEDPE | Fc sequence with |
| | VKFNWYVDGVEVHNAKTKPREEQYNSTYRVVSVLTVLHQDWLNGKEYKCKVS | hole and LALAPS |
| | NKALSAPIEKTISKAKGQPREPQVYTLPPSRDELTKNQVSLSCAVKGFYPSD | mutations and |
| | IAVEWESNGQPENNYKTTPPVLDSDGSFFLVSKLTVDKSRWQQGNVFSCSVM | portion of human |
| | HEALHNHYTQKSLSLSPG | IgG1 hinge |
| | | sequence |
| 23 | APELLGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLTCLVKGFYPSDIAVEWESYGT | |
| | EWANYKTTPPVLDSDGSFFLYSKLTVTKEEWQQGFVFSCSVMHEALHNHYTQ | |
| | KSLSLSPGK | |
| 24 | APELLGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLTCLVKGFYPSDIAVEWESYGT | |
| | EWANYKTTPPVLDSDGSFFLYSKLTVTKEEWQQGFVFSCSVMHEALHNHYTQ | |
| | KSLSLSPG | |
| 25 | APELLGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLWCLVKGFYPSDIAVEWESYGT | with knob |
| | EWANYKTTPPVLDSDGSFFLYSKLTVTKEEWQQGFVFSCSVMHEALHNHYTQ | mutation |
| | KSLSLSPGK | |
| 26 | APELLGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLWCLVKGFYPSDIAVEWESYGT | with knob |
| | EWANYKTTPPVLDSDGSFFLYSKLTVTKEEWQQGFVFSCSVMHEALHNHYTQ | mutation |
| | KSLSLSPG | |
| 27 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLWCLVKGFYPSDIAVEWESYGT | with knob and |
| | EWANYKTTPPVLDSDGSFFLYSKLTVTKEEWQQGFVFSCSVMHEALHNHYTQ | LALA mutations |
| | KSLSLSPGK | |
| 28 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLWCLVKGFYPSDIAVEWESYGT | with knob and |
| | EWANYKTTPPVLDSDGSFFLYSKLTVTKEEWQQGFVFSCSVMHEALHNHYTQ | LALA mutations |
| | KSLSLSPG | |
| 29 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALGAPIEK | CH3C.35.23.2 |
| | TISKAKGQPREPQVYTLPPSRDELTKNQVSLWCLVKGFYPSDIAVEWESYGT | with knob and |
| | EWANYKTTPPVLDSDGSFFLYSKLTVTKEEWQQGFVFSCSVMHEALHNHYTQ | LALAPG mutations |
| | KSLSLSPGK | |
| 30 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALGAPIEK | CH3C.35.23.2 |
| | TISKAKGQPREPQVYTLPPSRDELTKNQVSLWCLVKGFYPSDIAVEWESYGT | with knob and |
| | EWANYKTTPPVLDSDGSFFLYSKLTVTKEEWQQGFVFSCSVMHEALHNHYTQ | LALAPG mutations |
| | KSLSLSPG | |
| 31 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALSAPIEK | CH3C.35.23.2 |
| | TISKAKGQPREPQVYTLPPSRDELTKNQVSLWCLVKGFYPSDIAVEWESYGT | with knob and |
| | EWANYKTTPPVLDSDGSFFLYSKLTVTKEEWQQGFVFSCSVMHEALHNHYTQ | LALAPS mutations |
| | KSLSLSPGK | |
| 32 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALSAPIEK | CH3C.35.23.2 |
| | TISKAKGQPREPQVYTLPPSRDELTKNQVSLWCLVKGFYPSDIAVEWESYGT | with knob and |
| | EWANYKTTPPVLDSDGSFFLYSKLTVTKEEWQQGFVFSCSVMHEALHNHYTQ | LALAPS mutations |
| | KSLSLSPG | |
| 33 | APELLGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLSCAVKGFYPSDIAVEWESYGT | with hole |
| | EWANYKTTPPVLDSDGSFFLVSKLTVTKEEWQQGFVFSCSVMHEALHNHYTQ | mutations |
| | KSLSLSPGK | |
| 34 | APELLGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLSCAVKGFYPSDIAVEWESYGT | with hole |
| | EWANYKTTPPVLDSDGSFFLVSKLTVTKEEWQQGFVFSCSVMHEALHNHYTQ | mutations |
| | KSLSLSPG | |
| 35 | DKTHTCPPCPAPEAAGGPSVFLFPPKPKDTLMISRTPEVTCVVVDVSHEDPE | Clone |
| | VKFNWYVDGVEVHNAKTKPREEQYNSTYRVVSVLTVLHQDWLNGKEYKCKVS | CH3C.35.23.2 |
| | NKALPAPIEKTISKAKGQPREPQVYTLPPSRDELTKNQVSLWCLVKGFYPSD | with knob and |
| | IAVEWESYGTEWANYKTTPPVLDSDGSFFLYSKLTVTKEEWQQGFVFSCSVM | LALA mutations |
| | HEALHNHYTQKSLSLSPGK | and portion of |
| | | human IgG1 hinge |
| | | sequence |
| 36 | DKTHTCPPCPAPEAAGGPSVFLFPPKPKDTLMISRTPEVTCVVVDVSHEDPE | Clone |
| | VKFNWYVDGVEVHNAKTKPREEQYNSTYRVVSVLTVLHQDWLNGKEYKCKVS | CH3C.35.23.2 |
| | NKALPAPIEKTISKAKGQPREPQVYTLPPSRDELTKNQVSLWCLVKGFYPSD | with knob and |
| | IAVEWESYGTEWANYKTTPPVLDSDGSFFLYSKLTVTKEEWQQGFVFSCSVM | LALA mutations |
| | HEALHNHYTQKSLSLSPG | and portion of |
| | | human IgG1 hinge |
| | | sequence |
| 37 | DKTHTCPPCPAPEAAGGPSVFLFPPKPKDTLMISRTPEVTCVVVDVSHEDPE | Clone |
| | VKFNWYVDGVEVHNAKTKPREEQYNSTYRVVSVLTVLHQDWLNGKEYKCKVS | CH3C.35.23.2 |
| | NKALSAPIEKTISKAKGQPREPQVYTLPPSRDELTKNQVSLWCLVKGFYPSD | with knob and |
| | IAVEWESYGTEWANYKTTPPVLDSDGSFFLYSKLTVTKEEWQQGFVFSCSVM | LALAPS mutations |
| | HEALHNHYTQKSLSLSPGK | and portion of |
| | | human IgG1 hinge |
| | | sequence |
| 38 | DKTHTCPPCPAPEAAGGPSVFLFPPKPKDTLMISRTPEVTCVVVDVSHEDPE | Clone |
| | VKFNWYVDGVEVHNAKTKPREEQYNSTYRVVSVLTVLHQDWLNGKEYKCKVS | CH3C.35.23.2 |
| | NKALSAPIEKTISKAKGQPREPQVYTLPPSRDELTKNQVSLWCLVKGFYPSD | with knob and |
| | IAVEWESYGTEWANYKTTPPVLDSDGSFFLYSKLTVTKEEWQQGFVFSCSVM | LALAPS mutations |
| | HEALHNHYTQKSLSLSPG | and portion of |
| | | human IgG1 hinge |
| | | sequence |
| 39 | MRPLRPRAALLALLASLLAAPPVAPAEAPHLVX₁VDAARALWPLRRFWRS | Full-length |
| | TGFCPPLPHSQADQYVLSWDQQLNLAYVGAVPHRGIKQVRTHWLLELVTT | human alpha-L- |
| | RGSTGRGLSYNFTHLDGYLDLLRENQLLPGFELMGSASGHFTDFEDKQQV | iduronidase |
| | FEWKDLVSSLARRYIGRYGLAHVSKWNFETWNEPDHHDFDNVSMTMQGFL | polypeptide |
| | NYYDACSEGLRAASPALRLGGPGDSFHTPPRSPLSWGLLRHCHDGTNFFT | sequence, |
| | GEAGVRLDYISLHRKGARSSISILEQEKVVAQQIRQLFPKFADTPIYNDE | wherein X₁ is H |
| | ADPLVGWSLPQPWRADVTYAAMVVKVIAQHQNLLLANTTSAFPYALLSND | or Q; and X₂ is A |
| | NAFLSYHPHPFAQRTLTARFQVNNTRPPHVQLLRKPVLTAMGLLALLDEE | or T. |
| | QLWAEVSQAGTVLDSNHTVGVLASAHRPQGPADAWRAAVLIYASDDTRAH | |
| | PNRSVAVTLRLRGVPPGPGLVYVTRYLDNGLCSPDGEWRRLGRPVFPTAE | |
| | QFRRMRAAEDPVAAAPRPLPAGGRLTLRPALRLPSLLLVHVCARPEKPPG | |
| | QVTRLRALPLTQGQLVLVWSDEHVGSKCLWTYEIQFSQDGKAYTPVSRKP | |
| | STFNLFVFSPDTGAVSGSYRVRX₂LDYWARPGPFSDPVPYLEVPVPRGPP | |
| | SPGNP | |
| 40 | EAPHLVX₁VDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGFLNYYDACSEGLRAASPALRLGGPGDSFHTPPRSPLS | polypeptide, |
| | WGLLRHCHDGTNFFTGEAGVRLDYISLHRKGARSSISILEQEKVVAQQIRQL | wherein X₁ is H |
| | FPKFADTPIYNDEADPLVGWSLPQPWRADVTYAAMVVKVIAQHQNLLLANTT | or Q; and X₂ is A |
| | SAFPYALLSNDNAFLSYHPHPFAQRTLTARFQVNNTRPPHVQLLRKPVLTAM | or T. |
| | GLLALLDEEQLWAEVSQAGTVLDSNHTVGVLASAHRPQGPADAWRAAVLIYA | |
| | SDDTRAHPNRSVAVTLRLRGVPPGPGLVYVTRYLDNGLCSPDGEWRRLGRPV | |
| | FPTAEQFRRMRAAEDPVAAAPRPLPAGGRLTLRPALRLPSLLLVHVCARPEK | |
| | PPGQVTRLRALPLTQGQLVLVWSDEHVGSKCLWTYEIQFSQDGKAYTPVSRK | |
| | PSTFNLFVFSPDTGAVSGSYRVRX₂LDYWARPGPFSDPVPYLEVPVPRGPPSP | |
| | GNP | |
| 41 | EAPHLVQVDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGELNYYDACSEGLRAASPALRLGGPGDSFHTPPRSPLS | polypeptide |
| | WGLLRHCHDGTNFFTGEAGVRLDYISLHRKGARSSISILEQEKVVAQQIRQL | sequence with |
| | FPKFADTPIYNDEADPLVGWSLPQPWRADVTYAAMVVKVIAQHQNLLLANTT | Q33 and T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | according to EU |
| | SDDTRAHPNRSVAVTLRLRGVPPGPGLVYVTRYLDNGLCSPDGEWRRLGRPV | numbering |
| | FPTAEQFRRMRAAEDPVAAAPRPLPAGGRLTLRPALRLPSLLLVHVCARPEK | |
| | PPGQVTRLRALPLTQGQLVLVWSDEHVGSKCLWTYEIQFSQDGKAYTPVSRK | |
| | PSTFNLFVFSPDTGAVSGSYRVRTLDYWARPGPFSDPVPYLEVPVPRGPPSP | |
| | GNP | |
| 42 | EAPHLVQVDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGELNYYDACSEGLRAASPALRLGGPGDSFHTPPRSPLS | polypeptide |
| | WGLLRHCHDGTNFFTGEAGVRLDYISLHRKGARSSISILEQEKVVAQQIRQL | sequence with |
| | FPKFADTPIYNDEADPLVGWSLPQPWRADVTYAAMVVKVIAQHQNLLLANTT | Q33 and A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | according to EU |
| | SDDTRAHPNRSVAVTLRLRGVPPGPGLVYVTRYLDNGLCSPDGEWRRLGRPV | numbering |
| | FPTAEQFRRMRAAEDPVAAAPRPLPAGGRLTLRPALRLPSLLLVHVCARPEK | |
| | PPGQVTRLRALPLTQGQLVLVWSDEHVGSKCLWTYEIQFSQDGKAYTPVSRK | |
| | PSTFNLFVFSPDTGAVSGSYRVRALDYWARPGPFSDPVPYLEVPVPRGPPSP | |
| | GNP | |
| 43 | EAPHLVHVDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGFLNYYDACSEGLRAASPALRLGGPGDSFHTPPRSPLS | polypeptide |
| | WGLLRHCHDGTNFFTGEAGVRLDYISLHRKGARSSISILEQEKVVAQQIRQL | sequence with |
| | FPKFADTPIYNDEADPLVGWSLPQPWRADVTYAAMVVKVIAQHQNLLLANTT | H33 and T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | according to EU |
| | SDDTRAHPNRSVAVTLRLRGVPPGPGLVYVTRYLDNGLCSPDGEWRRLGRPV | numbering |
| | FPTAEQFRRMRAAEDPVAAAPRPLPAGGRLTLRPALRLPSLLLVHVCARPEK | |
| | PPGQVTRLRALPLTQGQLVLVWSDEHVGSKCLWTYEIQFSQDGKAYTPVSRK | |
| | PSTFNLFVFSPDTGAVSGSYRVRTLDYWARPGPFSDPVPYLEVPVPRGPPSP | |
| | GNP | |
| 44 | EAPHLVHVDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGELNYYDACSEGLRAASPALRLGGPGDSFHTPPRSPLS | polypeptide |
| | WGLLRHCHDGTNFFTGEAGVRLDYISLHRKGARSSISILEQEKVVAQQIRQL | sequence with |
| | FPKFADTPIYNDEADPLVGWSLPQPWRADVTYAAMVVKVIAQHQNLLLANTT | H33 and A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | according to EU |
| | SDDTRAHPNRSVAVTLRLRGVPPGPGLVYVTRYLDNGLCSPDGEWRRLGRPV | numbering |
| | FPTAEQFRRMRAAEDPVAAAPRPLPAGGRLTLRPALRLPSLLLVHVCARPEK | |
| | PPGQVTRLRALPLTQGQLVLVWSDEHVGSKCLWTYEIQFSQDGKAYTPVSRK | |
| | PSTFNLFVFSPDTGAVSGSYRVRALDYWARPGPFSDPVPYLEVPVPRGPPSP | |
| | GNP | |
| 45 | EAPHLVX₁VDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence, |
| | SAFPYALLSNDNAFLSYHPHPFAQRTLTARFQVNNTRPPHVQLLRKPVLTAM | wherein X₁ is H |
| | GLLALLDEEQLWAEVSQAGTVLDSNHTVGVLASAHRPQGPADAWRAAVLIYA | or Q; and X₂ is A |
| | SDDTRAHPNRSVAVTLRLRGVPPGPGLVYVTRYLDNGLCSPDGEWRRLGRPV | or T. |
| | FPTAEQFRRMRAAEDPVAAAPRPLPAGGRLTLRPALRLPSLLLVHVCARPEK | |
| | PPGQVTRLRALPLTQGQLVLVWSDEHVGSKCLWTYEIQFSQDGKAYTPVSRK | |
| | PSTFNLFVFSPDTGAVSGSYRVRX₂LDYWARPGPFSDPVPYLEVPV | |
| 46 | EAPHLVQVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | Q33 and T622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRTLDYWARPGPFSDPVPYLEVPV | |
| 47 | EAPHLVQVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | Q33 and A622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRALDYWARPGPFSDPVPYLEVPV | |
| 48 | EAPHLVHVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGFLNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | H33 and T622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRTLDYWARPGPFSDPVPYLEVPV | |
| 49 | EAPHLVHVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | H33 and A622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRALDYWARPGPFSDPVPYLEVPV | |
| 50 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVESPDTGAVSGSYRVRTLDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALA |
| | DVSHEDPEVKFNWYVDGVEVHNAKTKPREEQYNSTYRVVSVLTVLHQDWING | mutations |
| | KEYKCKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPGK | |
| 51 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVESPDTGAVSGSYRVRTLDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALA |
| | DVSHEDPEVKFNWYVDGVEVHNAKTKPREEQYNSTYRVVSVLTVLHQDWING | mutations |
| | KEYKCKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPG | |
| 52 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALA |
| | DVSHEDPEVKFNWYVDGVEVHNAKTKPREEQYNSTYRVVSVLTVLHQDWING | mutations |
| | KEYKCKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPGK | |
| 53 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALA |
| | DVSHEDPEVKFNWYVDGVEVHNAKTKPREEQYNSTYRVVSVLTVLHQDWING | mutations |
| | KEYKCKVSNKALPAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPG | |
| 54 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALA |
| | FNWYVDGVEVHNAKTKPREEQYNSTYRVVSVLTVLHQDWLNGKEYKCKVSNK | mutations |
| | ALPAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPGK | |
| 55 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALA |
| | FNWYVDGVEVHNAKTKPREEQYNSTYRVVSVLTVLHQDWLNGKEYKCKVSNK | mutations |
| | ALPAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPG | |
| 56 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALA |
| | FNWYVDGVEVHNAKTKPREEQYNSTYRVVSVLTVLHQDWLNGKEYKCKVSNK | mutations |
| | ALPAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPGK | |
| 57 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALA |
| | FNWYVDGVEVHNAKTKPREEQYNSTYRVVSVLTVLHQDWLNGKEYKCKVSNK | mutations |
| | ALPAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPG | |
| 58 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALAPS |
| | DVSHEDPEVKFNWYVDGVEVHNAKTKPREEQYNSTYRVVSVLTVLHQDWING | mutations |
| | KEYKCKVSNKALSAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPGK | |
| 59 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALAPS |
| | DVSHEDPEVKFNWYVDGVEVHNAKTKPREEQYNSTYRVVSVLTVLHQDWING | mutations |
| | KEYKCKVSNKALSAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPG | |
| 60 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALAPS |
| | DVSHEDPEVKFNWYVDGVEVHNAKTKPREEQYNSTYRVVSVLTVLHQDWLNG | mutations |
| | KEYKCKVSNKALSAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPGK | |
| 61 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGPPSP | Fc sequence with |
| | GNPGGGGSDKTHTCPPCPAPEAAGGPSVELFPPKPKDTLMISRTPEVTCVVV | hole and LALAPS |
| | DVSHEDPEVKFNWYVDGVEVHNAKTKPREEQYNSTYRVVSVLTVLHQDWING | mutations |
| | KEYKCKVSNKALSAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPG | |
| 62 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALAPS |
| | FNWYVDGVEVHNAKTKPREEQYNSTYRVVSVLTVLHQDWLNGKEYKCKVSNK | mutations |
| | ALSAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPGK | |
| 63 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALAPS |
| | FNWYVDGVEVHNAKTKPREEQYNSTYRVVSVLTVLHQDWLNGKEYKCKVSNK | mutations |
| | ALSAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPG | |
| 64 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALAPS |
| | FNWYVDGVEVHNAKTKPREEQYNSTYRVVSVLTVLHQDWLNGKEYKCKVSNK | mutations |
| | ALSAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPGK | |
| 65 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVGGGGSDK | Fc sequence with |
| | THTCPPCPAPEAAGGPSVFLFPPKPKDTLMISRTPEVTCVVVDVSHEDPEVK | hole and LALAPS |
| | FNWYVDGVEVHNAKTKPREEQYNSTYRVVSVLTVLHQDWLNGKEYKCKVSNK | mutations |
| | ALSAPIEKTISKAKGQPREPQVYTLPPSRDELTKNQVSLSCAVKGFYPSDIA | |
| | VEWESNGQPENNYKTTPPVLDSDGSFFLVSKLTVDKSRWQQGNVFSCSVMHE | |
| | ALHNHYTQKSLSLSPG | |
| 66 | DKTHTCPPCPAPEAAGGPSVFLFPPKPKDTLMISRTPEVTCVVVDVSHEDPE | Fc-IDUA fusion |
| | VKFNWYVDGVEVHNAKTKPREEQYNSTYRVVSVLTVLHQDWLNGKEYKCKVS | polypeptide with |
| | NKALPAPIEKTISKAKGQPREPQVYTLPPSRDELTKNQVSLSCAVKGFYPSD | mature human |
| | IAVEWESNGQPENNYKTTPPVLDSDGSFFLVSKLTVDKSRWQQGNVFSCSVM | IDUA sequence |
| | HEALHNHYTQKSLSLSPGKGGGGSEAPHLVQVDAARALWPLRRFWRSTGFCP | with Q33 and |
| | PLPHSQADQYVLSWDQQLNLAYVGAVPHRGIKQVRTHWLLELVTTRGSTGRG | A622 |
| | LSYNFTHLDGYLDLLRENQLLPGFELMGSASGHFTDFEDKQQVFEWKDLVSS | (underlined) |
| | LARRYIGRYGLAHVSKWNFETWNEPDHHDFDNVSMTMQGFLNYYDACSEGLR | with G₄S linker |
| | AASPALRLGGPGDSFHTPPRSPLSWGLLRHCHDGTNFFTGEAGVRLDYISLH | (SEQ ID NO: 72) |
| | RKGARSSISILEQEKVVAQQIRQLFPKFADTPIYNDEADPLVGWSLPQPWRA | fused to the C- |
| | DVTYAAMVVKVIAQHQNLLLANTTSAFPYALLSNDNAFLSYHPHPFAQRTLT | terminus of an |
| | ARFQVNNTRPPHVQLLRKPVLTAMGLLALLDEEQLWAEVSQAGTVLDSNHTV | Fc sequence with |
| | GVLASAHRPQGPADAWRAAVLIYASDDTRAHPNRSVAVTLRLRGVPPGPGLV | hole and LALA |
| | YVTRYLDNGLCSPDGEWRRLGRPVEPTAEQFRRMRAAEDPVAAAPRPLPAGG | mutations |
| | RLTLRPALRLPSLLLVHVCARPEKPPGQVTRLRALPLTQGQLVLVWSDEHVG | |
| | SKCLWTYEIQFSQDGKAYTPVSRKPSTFNLFVFSPDTGAVSGSYRVRALDYW | |
| | ARPGPFSDPVPYLEVPVPRGPPSPGNP | |
| 67 | DKTHTCPPCPAPEAAGGPSVFLFPPKPKDTLMISRTPEVTCVVVDVSHEDPE | Fc-IDUA fusion |
| | VKFNWYVDGVEVHNAKTKPREEQYNSTYRVVSVLTVLHQDWLNGKEYKCKVS | polypeptide with |
| | NKALPAPIEKTISKAKGQPREPQVYTLPPSRDELTKNQVSLSCAVKGFYPSD | mature human |
| | IAVEWESNGQPENNYKTTPPVLDSDGSFFLVSKLTVDKSRWQQGNVFSCSVM | IDUA sequence |
| | HEALHNHYTQKSLSLSPGKGGGGSEAPHLVQVDAARALWPLRRFWRSTGFCP | with Q33 and |
| | PLPHSQADQYVLSWDQQLNLAYVGAVPHRGIKQVRTHWLLELVTTRGSTGRG | T622 |
| | LSYNFTHLDGYLDLLRENQLLPGFELMGSASGHFTDFEDKQQVFEWKDLVSS | (underlined) |
| | LARRYIGRYGLAHVSKWNFETWNEPDHHDEDNVSMTMQGELNYYDACSEGLR | with G₄S linker |
| | AASPALRLGGPGDSFHTPPRSPLSWGLLRHCHDGTNFFTGEAGVRLDYISLH | (SEQ ID NO: 72) |
| | RKGARSSISILEQEKVVAQQIRQLFPKFADTPIYNDEADPLVGWSLPQPWRA | fused to the C- |
| | DVTYAAMVVKVIAQHQNLLLANTTSAFPYALLSNDNAFLSYHPHPFAQRTLT | terminus of an |
| | ARFQVNNTRPPHVQLLRKPVLTAMGLLALLDEEQLWAEVSQAGTVLDSNHTV | Fc sequence with |
| | GVLASAHRPQGPADAWRAAVLIYASDDTRAHPNRSVAVTLRLRGVPPGPGLV | hole and LALA |
| | YVTRYLDNGLCSPDGEWRRLGRPVFPTAEQFRRMRAAEDPVAAAPRPLPAGG | mutations |
| | RLTLRPALRLPSLLLVHVCARPEKPPGQVTRLRALPLTQGQLVLVWSDEHVG | |
| | SKCLWTYEIQFSQDGKAYTPVSRKPSTFNLFVFSPDTGAVSGSYRVRTLDYW | |
| | ARPGPFSDPVPYLEVPVPRGPPSPGNP | |
| 68 | DKTHTCPPCPAPEAAGGPSVFLFPPKPKDTLMISRTPEVTCVVVDVSHEDPE | Fc-IDUA fusion |
| | VKFNWYVDGVEVHNAKTKPREEQYNSTYRVVSVLTVLHQDWLNGKEYKCKVS | polypeptide with |
| | NKALSAPIEKTISKAKGQPREPQVYTLPPSRDELTKNQVSLSCAVKGFYPSD | mature human |
| | IAVEWESNGQPENNYKTTPPVLDSDGSFFLVSKLTVDKSRWQQGNVFSCSVM | IDUA sequence |
| | HEALHNHYTQKSLSLSPGKGGGGSEAPHLVQVDAARALWPLRRFWRSTGFCP | with Q33 and |
| | PLPHSQADQYVLSWDQQLNLAYVGAVPHRGIKQVRTHWLLELVTTRGSTGRG | A622 |
| | LSYNFTHLDGYLDLLRENQLLPGFELMGSASGHFTDFEDKQQVFEWKDLVSS | (underlined) |
| | LARRYIGRYGLAHVSKWNFETWNEPDHHDFDNVSMTMQGELNYYDACSEGLR | with G₄S linker |
| | AASPALRLGGPGDSFHTPPRSPLSWGLLRHCHDGTNFFTGEAGVRLDYISLH | (SEQ ID NO: 72) |
| | RKGARSSISILEQEKVVAQQIRQLFPKFADTPIYNDEADPLVGWSLPQPWRA | fused to the C- |
| | DVTYAAMVVKVIAQHQNLLLANTTSAFPYALLSNDNAFLSYHPHPFAQRTLT | terminus of an |
| | ARFQVNNTRPPHVQLLRKPVLTAMGLLALLDEEQLWAEVSQAGTVLDSNHTV | Fc sequence with |
| | GVLASAHRPQGPADAWRAAVLIYASDDTRAHPNRSVAVTLRLRGVPPGPGLV | hole and LALAPS |
| | YVTRYLDNGLCSPDGEWRRLGRPVFPTAEQFRRMRAAEDPVAAAPRPLPAGG | mutations |
| | RLTLRPALRLPSLLLVHVCARPEKPPGQVTRLRALPLTQGQLVLVWSDEHVG | |
| | SKCLWTYEIQFSQDGKAYTPVSRKPSTFNLFVFSPDTGAVSGSYRVRALDYW | |
| | ARPGPFSDPVPYLEVPVPRGPPSPGNP | |
| 69 | DKTHTCPPCPAPEAAGGPSVFLFPPKPKDTLMISRTPEVTCVVVDVSHEDPE | Fc-IDUA fusion |
| | VKFNWYVDGVEVHNAKTKPREEQYNSTYRVVSVLTVLHQDWLNGKEYKCKVS | polypeptide with |
| | NKALSAPIEKTISKAKGQPREPQVYTLPPSRDELTKNQVSLSCAVKGFYPSD | mature human |
| | IAVEWESNGQPENNYKTTPPVLDSDGSFFLVSKLTVDKSRWQQGNVFSCSVM | IDUA sequence |
| | HEALHNHYTQKSLSLSPGKGGGGSEAPHLVQVDAARALWPLRRFWRSTGFCP | with Q33 and |
| | PLPHSQADQYVLSWDQQLNLAYVGAVPHRGIKQVRTHWLLELVTTRGSTGRG | T622 |
| | LSYNFTHLDGYLDLLRENQLLPGFELMGSASGHFTDFEDKQQVFEWKDLVSS | (underlined) |
| | LARRYIGRYGLAHVSKWNFETWNEPDHHDFDNVSMTMQGELNYYDACSEGLR | with G₄S linker |
| | AASPALRLGGPGDSFHTPPRSPLSWGLLRHCHDGTNFFTGEAGVRLDYISLH | (SEQ ID NO: 72) |
| | RKGARSSISILEQEKVVAQQIRQLFPKFADTPIYNDEADPLVGWSLPQPWRA | fused to the C- |
| | DVTYAAMVVKVIAQHQNLLLANTTSAFPYALLSNDNAFLSYHPHPFAQRTLT | terminus of an |
| | ARFQVNNTRPPHVQLLRKPVLTAMGLLALLDEEQLWAEVSQAGTVLDSNHTV | Fc sequence with |
| | GVLASAHRPQGPADAWRAAVLIYASDDTRAHPNRSVAVTLRLRGVPPGPGLV | hole and LALAPS |
| | YVTRYLDNGLCSPDGEWRRLGRPVFPTAEQFRRMRAAEDPVAAAPRPLPAGG | mutations |
| | RLTLRPALRLPSLLLVHVCARPEKPPGQVTRLRALPLTQGQLVLVWSDEHVG | |
| | SKCLWTYEIQFSQDGKAYTPVSRKPSTFNLFVFSPDTGAVSGSYRVRTLDYW | |
| | ARPGPFSDPVPYLEVPVPRGPPSPGNP | |
| 70 | MGWSCIILFLVATATGAYA | Secretion signal |
| | | peptide |
| | GS | GS linker |
| 72 | GGGGS | Glycine-rich |
| | | linker |
| 73 | GGGGSGGGGS | Glycine-rich |
| | | linker |
| 74 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | knob and LALA |
| | TISKAKGQPREPQVYTLPPSRDELTKNQVSLWCLVKGFYPSDIAVEWESNGQ | mutations |
| | PENNYKTTPPVLDSDGSFFLYSKLTVDKSRWQQGNVFSCSVMHEALHNHYTQ | |
| | KSLSLSPGK | |
| 75 | APEAAGGPSVFLFPPKPKDTLMISRTPEVTCVVVDVSHEDPEVKFNWYVDGV | Fc sequence with |
| | EVHNAKTKPREEQYNSTYRVVSVLTVLHQDWLNGKEYKCKVSNKALPAPIEK | knob and LALA |
| | TISKAKGQPREPQVYTLPPSRDELTKNQVSLWCLVKGFYPSDIAVEWESNGQ | mutations |
| | PENNYKTTPPVLDSDGSFFLYSKLTVDKSRWQQGNVFSCSVMHEALHNHYTQ | |
| | KSLSLSPG | |
| 76 | DKTHTCPPCPAPEAAGGPSVFLFPPKPKDTLMISRTPEVTCVVVDVSHEDPE | Fc sequence with |
| | VKFNWYVDGVEVHNAKTKPREEQYNSTYRVVSVLTVLHQDWLNGKEYKCKVS | knob and LALA |
| | NKALPAPIEKTISKAKGQPREPQVYTLPPSRDELTKNQVSLWCLVKGFYPSD | mutations and |
| | IAVEWESNGQPENNYKTTPPVLDSDGSFFLYSKLTVDKSRWQQGNVFSCSVM | portion of human |
| | HEALHNHYTQKSLSLSPGK | IgG1 hinge |
| | | sequence |
| 77 | DKTHTCPPCPAPEAAGGPSVFLFPPKPKDTLMISRTPEVTCVVVDVSHEDPE | Fc sequence with |
| | VKFNWYVDGVEVHNAKTKPREEQYNSTYRVVSVLTVLHQDWLNGKEYKCKVS | knob and LALA |
| | NKALPAPIEKTISKAKGQPREPQVYTLPPSRDELTKNQVSLWCLVKGFYPSD | mutations and |
| | IAVEWESNGQPENNYKTTPPVLDSDGSFFLYSKLTVDKSRWQQGNVFSCSVM | portion of human |
| | HEALHNHYTQKSLSLSPG | IgG1 hinge |
| | | sequence |
| 78 | EAPHLVX₁VDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence, |
| | SAFPYALLSNDNAFLSYHPHPFAQRTLTARFQVNNTRPPHVQLLRKPVLTAM | wherein X₁ is H |
| | GLLALLDEEQLWAEVSQAGTVLDSNHTVGVLASAHRPQGPADAWRAAVLIYA | or Q; and X₂ is A |
| | SDDTRAHPNRSVAVTLRLRGVPPGPGLVYVTRYLDNGLCSPDGEWRRLGRPV | or T. |
| | FPTAEQFRRMRAAEDPVAAAPRPLPAGGRLTLRPALRLPSLLLVHVCARPEK | |
| | PPGQVTRLRALPLTQGQLVLVWSDEHVGSKCLWTYEIQFSQDGKAYTPVSRK | |
| | PSTFNLFVFSPDTGAVSGSYRVRX₂LDYWARPGPFSDPVPYLEVPVPRG | |
| 79 | EAPHLVQVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | Q33 and T622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRTLDYWARPGPFSDPVPYLEVPVPRG | |
| 80 | EAPHLVQVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | Q33 and A622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRALDYWARPGPFSDPVPYLEVPVPRG | |
| 81 | EAPHLVHVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGFLNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | H33 and T622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRTLDYWARPGPFSDPVPYLEVPVPRG | |
| 82 | EAPHLVHVDAARALWPLRRFWRSTGFCPPLPHSQADQYVLSWDQQLNLAYVG | Embodiment of a |
| | AVPHRGIKQVRTHWLLELVTTRGSTGRGLSYNFTHLDGYLDLLRENQLLPGF | truncated human |
| | ELMGSASGHFTDFEDKQQVFEWKDLVSSLARRYIGRYGLAHVSKWNFETWNE | alpha-L- |
| | PDHHDFDNVSMTMQGELNYYDACSEGLRAASPALRLGGPGDSFHTPPRSPLS | iduronidase |
| | WGLLRHCHDGTNFFTGEAGVRLDYISLHRKGARSSISILEQEKVVAQQIRQL | polypeptide |
| | FPKFADTPIYNDEADPLVGWSLPQPWRADVTYAAMVVKVIAQHQNLLLANTT | sequence with |
| | SAFPYALLSNDNAFLSYHPHPFAQRTLTARFQVNNTRPPHVQLLRKPVLTAM | H33 and A622 |
| | GLLALLDEEQLWAEVSQAGTVLDSNHTVGVLASAHRPQGPADAWRAAVLIYA | (underlined), |
| | SDDTRAHPNRSVAVTLRLRGVPPGPGLVYVTRYLDNGLCSPDGEWRRLGRPV | according to EU |
| | FPTAEQFRRMRAAEDPVAAAPRPLPAGGRLTLRPALRLPSLLLVHVCARPEK | numbering |
| | PPGQVTRLRALPLTQGQLVLVWSDEHVGSKCLWTYEIQFSQDGKAYTPVSRK | |
| | PSTFNLFVFSPDTGAVSGSYRVRALDYWARPGPFSDPVPYLEVPVPRG | |
| 83 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALA |
| | EVKFNWYVDGVEVHNAKTKPREEQYNSTYRVVSVLTVLHQDWLNGKEYKCKV | mutations |
| | SNKALPAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPGK | |
| 84 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALA |
| | EVKFNWYVDGVEVHNAKTKPREEQYNSTYRVVSVLTVLHQDWLNGKEYKCKV | mutations |
| | SNKALPAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPG | |
| 85 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALA |
| | EVKFNWYVDGVEVHNAKTKPREEQYNSTYRVVSVLTVLHQDWLNGKEYKCKV | mutations |
| | SNKALPAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPGK | |
| 86 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALA |
| | EVKFNWYVDGVEVHNAKTKPREEQYNSTYRVVSVLTVLHQDWLNGKEYKCKV | mutations |
| | SNKALPAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPG | |
| 87 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALAPS |
| | EVKFNWYVDGVEVHNAKTKPREEQYNSTYRVVSVLTVLHQDWLNGKEYKCKV | mutations |
| | SNKALSAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPGK | |
| 88 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G4S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALAPS |
| | EVKFNWYVDGVEVHNAKTKPREEQYNSTYRVVSVLTVLHQDWLNGKEYKCKV | mutations |
| | SNKALSAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPG | |
| 89 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALAPS |
| | EVKFNWYVDGVEVHNAKTKPREEQYNSTYRVVSVLTVLHQDWLNGKEYKCKV | mutations |
| | SNKALSAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPGK | |
| 90 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | truncated human |
| | PDHHDEDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGGGGG | Fc sequence with |
| | SDKTHTCPPCPAPEAAGGPSVFLFPPKPKDTLMISRTPEVTCVVVDVSHEDP | hole and LALAPS |
| | EVKFNWYVDGVEVHNAKTKPREEQYNSTYRVVSVLTVLHQDWLNGKEYKCKV | mutations |
| | SNKALSAPIEKTISKAKGQPREPQVYTLPPSRDELTKNQVSLSCAVKGFYPS | |
| | DIAVEWESNGQPENNYKTTPPVLDSDGSFFLVSKLTVDKSRWQQGNVFSCSV | |
| | MHEALHNHYTQKSLSLSPG | |
| 91 | EAPHLVHVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with H33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALAPS |
| | DVSHEDPEVKFNWYVDGVEVHNAKTKPREEQYNSTYRVVSVLTVLHQDWING | mutations |
| | KEYKCKVSNKALSAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPGK | |
| 92 | EAPHLVHVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with H33 and |
| | FPKFADTPIYNDEADPLVGWSLPQPWRADVTYAAMVVKVIAQHQNLLLANTT | A622 |
| | SAFPYALLSNDNAFLSYHPHPFAQRTLTARFQVNNTRPPHVQLLRKPVLTAM | (underlined), |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRALDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | hole and LALAPS |
| | DVSHEDPEVKFNWYVDGVEVHNAKTKPREEQYNSTYRVVSVLTVLHQDWING | mutations |
| | KEYKCKVSNKALSAPIEKTISKAKGQPREPQVYTLPPSRDELTKNQVSLSCA | |
| | VKGFYPSDIAVEWESNGQPENNYKTTPPVLDSDGSFFLVSKLTVDKSRWQQG | |
| | NVFSCSVMHEALHNHYTQKSLSLSPG | |
| 93 | EAPHLVQVDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGFLNYYDACSEGLRAASPALRLGGPGDSFHTPPRSPLS | polypeptide |
| | WGLLRHCHDGTNFFTGEAGVRLDYISLHRKGARSSISILEQEKVVAQQIRQL | sequence with |
| | FPKFADTPIYNDEADPLVGWSLPQPWRADVTYAAMVVKVIAQHQNLLLANTT | Q33 and A622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | fused to a 6x |
| | SDDTRAHPNRSVAVTLRLRGVPPGPGLVYVTRYLDNGLCSPDGEWRRLGRPV | His tag (SEQ ID |
| | FPTAEQFRRMRAAEDPVAAAPRPLPAGGRLTLRPALRLPSLLLVHVCARPEK | NO: 96) |
| | PPGQVTRLRALPLTQGQLVLVWSDEHVGSKCLWTYEIQFSQDGKAYTPVSRK | |
| | PSTFNLFVESPDTGAVSGSYRVRALDYWARPGPFSDPVPYLEVPVPRGPPSP | |
| | GNPGSHHHHHH | |
| 94 | EAPHLVQVDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGELNYYDACSEGLRAASPALRLGGPGDSFHTPPRSPLS | polypeptide |
| | WGLLRHCHDGTNFFTGEAGVRLDYISLHRKGARSSISILEQEKVVAQQIRQL | sequence with |
| | FPKFADTPIYNDEADPLVGWSLPQPWRADVTYAAMVVKVIAQHQNLLLANTT | Q33 and T622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | fused to a 6x |
| | SDDTRAHPNRSVAVTLRLRGVPPGPGLVYVTRYLDNGLCSPDGEWRRLGRPV | His tag (SEQ ID |
| | FPTAEQFRRMRAAEDPVAAAPRPLPAGGRLTLRPALRLPSLLLVHVCARPEK | NO: 96) |
| | PPGQVTRLRALPLTQGQLVLVWSDEHVGSKCLWTYEIQFSQDGKAYTPVSRK | |
| | PSTFNLFVESPDTGAVSGSYRVRTLDYWARPGPFSDPVPYLEVPVPRGPPSP | |
| | GNPGSHHHHHH | |
| 95 | X₃APHLVHVDAARALWPLRRFWRSTGFCPPLPHSQADQYVLSWDQQLNLAYVG | Mature human |
| | AVPHRGIKQVRTHWLLELVTTRGSTGRGLSYNFTHLDGYLDLLRENQLLPGF | alpha-L- |
| | ELMGSASGHFTDFEDKQQVFEWKDLVSSLARRYIGRYGLAHVSKWNFETWNE | iduronidase |
| | PDHHDFDNVSMTMQGFLNYYDACSEGLRAASPALRLGGPGDSFHTPPRSPLS | polypeptide |
| | WGLLRHCHDGTNFFTGEAGVRLDYISLHRKGARSSISILEQEKVVAQQIRQL | sequence with |
| | FPKFADTPIYNDEADPLVGWSLPQPWRADVTYAAMVVKVIAQHQNLLLANTT | H33 and A622, |
| | SAFPYALLSNDNAFLSYHPHPFAQRTLTARFQVNNTRPPHVQLLRKPVLTAM | wherein X₃ is E |
| | GLLALLDEEQLWAEVSQAGTVLDSNHTVGVLASAHRPQGPADAWRAAVLIYA | or absent |
| | SDDTRAHPNRSVAVTLRLRGVPPGPGLVYVTRYLDNGLCSPDGEWRRLGRPV | (underlined) |
| | FPTAEQFRRMRAAEDPVAAAPRPLPAGGRLTLRPALRLPSLLLVHVCARPEK | fused to a 6x |
| | PPGQVTRLRALPLTQGQLVLVWSDEHVGSKCLWTYEIQFSQDGKAYTPVSRK | His tag (SEQ ID |
| | PSTFNLFVFSPDTGAVSGSYRVRALDYWARPGPFSDPVPYLEVPVPRGPPSP | NO: 96) |
| | GNPGSHHHHHH | |
| 96 | HHHHHH | 6x His tag |
| 97 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLSCAVKGFYPSDIAVEWESYGT | with hole and |
| | EWANYKTTPPVLDSDGSFFLVSKLTVTKEEWQQGFVFSCSVMHEALHNHYTQ | LALA mutations |
| | KSLSLSPGK | |
| 98 | APEAAGGPSVFLFPPKPKDTLMISRTPEVTCVVVDVSHEDPEVKFNWYVDGV | Clone |
| | EVHNAKTKPREEQYNSTYRVVSVLTVLHQDWLNGKEYKCKVSNKALPAPIEK | CH3C.35.23.2 |
| | TISKAKGQPREPQVYTLPPSRDELTKNQVSLSCAVKGFYPSDIAVEWESYGT | with hole and |
| | EWANYKTTPPVLDSDGSFFLVSKLTVTKEEWQQGFVFSCSVMHEALHNHYTQ | LALA mutations |
| | KSLSLSPG | |
| 99 | X₃APHLVX₁VDAARALWPLRRFWRSTGFCPPLPHSQADQYVLSWDQQLNLAYV | Mature human |
| | GAVPHRGIKQVRTHWLLELVTTRGSTGRGLSYNFTHLDGYLDLLRENQLLPG | alpha-L- |
| | FELMGSASGHFTDFEDKQQVFEWKDLVSSLARRYIGRYGLAHVSKWNFETWN | iduronidase |
| | EPDHHDFDNVSMTMQGELNYYDACSEGLRAASPALRLGGPGDSFHTPPRSPL | polypeptide, |
| | SWGLLRHCHDGTNFFTGEAGVRLDYISLHRKGARSSISILEQEKVVAQQIRQ | wherein X₁ is H |
| | LFPKFADTPIYNDEADPLVGWSLPQPWRADVTYAAMVVKVIAQHQNLLLANT | or Q; X₂ is A or |
| | TSAFPYALLSNDNAFLSYHPHPFAQRTLTARFQVNNTRPPHVQLLRKPVLTA | T; and X₃ is E or |
| | MGLLALLDEEQLWAEVSQAGTVLDSNHTVGVLASAHRPQGPADAWRAAVLIY | absent. |
| | ASDDTRAHPNRSVAVTLRLRGVPPGPGLVYVTRYLDNGLCSPDGEWRRLGRP | |
| | VEPTAEQFRRMRAAEDPVAAAPRPLPAGGRLTLRPALRLPSLLLVHVCARPE | |
| | KPPGQVTRLRALPLTQGQLVLVWSDEHVGSKCLWTYEIQFSQDGKAYTPVSR | |
| | KPSTFNLFVFSPDTGAVSGSYRVRX₂LDYWARPGPFSDPVPYLEVPVPRGPPS | |
| | PGNP | |
| 100 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | knob and LALA |
| | DVSHEDPEVKFNWYVDGVEVHNAKTKPREEQYNSTYRVVSVLTVLHQDWING | mutations |
| | KEYKCKVSNKALPAPIEKTISKAKGQPREPQVYTLPPSRDELTKNQVSLWCL | |
| | VKGFYPSDIAVEWESNGQPENNYKTTPPVLDSDGSFFLYSKLTVDKSRWQQG | |
| | NVFSCSVMHEALHNHYTQKSLSLSPG | |
| 101 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of |
| | PSTFNLFVFSPDTGAVSGSYRVRTLDYWARPGPFSDPVPYLEVPVPRGPPSP | clone |
| | GNPGGGGSDKTHTCPPCPAPEAAGGPSVFLFPPKPKDTLMISRTPEVTCVVV | CH3C.35.23.2 |
| | DVSHEDPEVKFNWYVDGVEVHNAKTKPREEQYNSTYRVVSVLTVLHQDWING | with knob and |
| | KEYKCKVSNKALPAPIEKTISKAKGQPREPQVYTLPPSRDELTKNQVSLWCL | LALA mutations |
| | VKGFYPSDIAVEWESYGTEWANYKTTPPVLDSDGSFFLYSKLTVTKEEWQQG | |
| | FVFSCSVMHEALHNHYTQKSLSLSPGK | |
| 102 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGELNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of |
| | PSTFNLFVESPDTGAVSGSYRVRTLDYWARPGPFSDPVPYLEVPVPRGPPSP | clone |
| | GNPGGGGSDKTHTCPPCPAPEAAGGPSVFLFPPKPKDTLMISRTPEVTCVVV | CH3C.35.23.2 |
| | DVSHEDPEVKFNWYVDGVEVHNAKTKPREEQYNSTYRVVSVLTVLHQDWING | with knob and |
| | KEYKCKVSNKALPAPIEKTISKAKGQPREPQVYTLPPSRDELTKNQVSLWCL | LALA mutations |
| | VKGFYPSDIAVEWESYGTEWANYKTTPPVLDSDGSFFLYSKLTVTKEEWQQG | |
| | FVFSCSVMHEALHNHYTQKSLSLSPG | |
| 103 | DKTHTCPPCPAPEAAGGPSVFLFPPKPKDTLMISRTPEVTCVVVDVSHEDPE | Fc-IDUA fusion |
| | VKFNWYVDGVEVHNAKTKPREEQYNSTYRVVSVLTVLHQDWLNGKEYKCKVS | polypeptide with |
| | NKALPAPIEKTISKAKGQPREPQVYTLPPSRDELTKNQVSLWCLVKGFYPSD | mature human |
| | IAVEWESYGTEWANYKTTPPVLDSDGSFFLYSKLTVTKEEWQQGFVFSCSVM | IDUA sequence |
| | HEALHNHYTQKSLSLSPGKGGGGSEAPHLVQVDAARALWPLRRFWRSTGFCP | with Q33 and |
| | PLPHSQADQYVLSWDQQLNLAYVGAVPHRGIKQVRTHWLLELVTTRGSTGRG | T622 |
| | LSYNFTHLDGYLDLLRENQLLPGFELMGSASGHFTDFEDKQQVFEWKDLVSS | (underlined) |
| | LARRYIGRYGLAHVSKWNFETWNEPDHHDEDNVSMTMQGELNYYDACSEGLR | with G₄S linker |
| | AASPALRLGGPGDSFHTPPRSPLSWGLLRHCHDGTNFFTGEAGVRLDYISLH | (SEQ ID NO: 72) |
| | RKGARSSISILEQEKVVAQQIRQLFPKFADTPIYNDEADPLVGWSLPQPWRA | fused to the C- |
| | DVTYAAMVVKVIAQHQNLLLANTTSAFPYALLSNDNAFLSYHPHPFAQRTLT | terminus of |
| | ARFQVNNTRPPHVQLLRKPVLTAMGLLALLDEEQLWAEVSQAGTVLDSNHTV | clone |
| | GVLASAHRPQGPADAWRAAVLIYASDDTRAHPNRSVAVTLRLRGVPPGPGLV | CH3C.35.23.2 |
| | YVTRYLDNGLCSPDGEWRRLGRPVFPTAEQFRRMRAAEDPVAAAPRPLPAGG | with knob and |
| | RLTLRPALRLPSLLLVHVCARPEKPPGQVTRLRALPLTQGQLVLVWSDEHVG | LALA mutations |
| | SKCLWTYEIQFSQDGKAYTPVSRKPSTFNLFVFSPDTGAVSGSYRVRTLDYW | |
| | ARPGPFSDPVPYLEVPVPRGPPSPGNP | |
| 104 | EAPHLVQVDAARALWPLRRFWRSTGFCPPLPHSQADQYVLSWDQQLNLAYVG | IDUA-Fc fusion |
| | AVPHRGIKQVRTHWLLELVTTRGSTGRGLSYNFTHLDGYLDLLRENQLLPGF | polypeptide with |
| | ELMGSASGHFTDFEDKQQVFEWKDLVSSLARRYIGRYGLAHVSKWNFETWNE | mature human |
| | PDHHDFDNVSMTMQGFLNYYDACSEGLRAASPALRLGGPGDSFHTPPRSPLS | IDUA sequence |
| | WGLLRHCHDGTNFFTGEAGVRLDYISLHRKGARSSISILEQEKVVAQQIRQL | with Q33 and |
| | FPKFADTPIYNDEADPLVGWSLPQPWRADVTYAAMVVKVIAQHQNLLLANTT | T622 |
| | SAFPYALLSNDNAFLSYHPHPFAQRTLTARFQVNNTRPPHVQLLRKPVLTAM | (underlined) |
| | GLLALLDEEQLWAEVSQAGTVLDSNHTVGVLASAHRPQGPADAWRAAVLIYA | with G₄S linker |
| | SDDTRAHPNRSVAVTLRLRGVPPGPGLVYVTRYLDNGLCSPDGEWRRLGRPV | (SEQ ID NO: 72) |
| | FPTAEQFRRMRAAEDPVAAAPRPLPAGGRLTLRPALRLPSLLLVHVCARPEK | fused to the N- |
| | PPGQVTRLRALPLTQGQLVLVWSDEHVGSKCLWTYEIQFSQDGKAYTPVSRK | terminus of an |
| | PSTFNLFVFSPDTGAVSGSYRVRTLDYWARPGPFSDPVPYLEVPVPRGPPSP | Fc sequence with |
| | GNPGGGGSDKTHTCPPCPAPEAAGGPSVFLFPPKPKDTLMISRTPEVTCVVV | knob and LALA |
| | DVSHEDPEVKFNWYVDGVEVHNAKTKPREEQYNSTYRVVSVLTVLHQDWING | mutations |
| | KEYKCKVSNKALPAPIEKTISKAKGQPREPQVYTLPPSRDELTKNQVSLWCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |

All publications, patents, and patent documents are incorporated by reference herein, as though individually incorporated by reference. The present disclosure has been described with reference to various specific and preferred embodiments and techniques. However, it should be understood that many variations and modifications may be made while remaining within the spirit and scope of the invention.

1. A protein comprising:
a. a first Fc polypeptide linked to an alpha-L-iduronidase (IDUA) amino acid sequence, an IDUA variant amino acid sequence, or a catalytically active fragment thereof; and
b. a second Fc polypeptide that comprises a sequence having at least 90% identity to SEQ ID NO: 28 and that is capable of specifically binding to a transferrin receptor (TfR).

2-3. (canceled)

4. The protein of claim 1, wherein the second Fc polypeptide comprises at the following positions, according to EU numbering:
i. Glu at position 380;
ii. Tyr at position 384;
iii. Thr at position 386;
iv. Glu at position 387;
v. Trp at position 388;
vi. Ala at position 389;
vii. Asn at position 390;
viii. Thr at position 413;
ix. Glu at position 415;
x. Glu at position 416; and
xi. Phe at position 421.

5-9. (canceled)

10. The protein of claim 1, wherein the IDUA amino acid sequence comprises an amino acid sequence having at least 80%, 85%, 90%, or 95% identity to any one of SEQ ID NOS: 39, 40, 45, 78, and 99.

11-12. (canceled)

13. The protein of claim 1, wherein the IDUA amino acid sequence comprises the amino acid sequence of any one of SEQ ID NOS: 41-44, 46-49 and 79-82.

14-17. (canceled)

18. The protein of claim 1, wherein the first Fc polypeptide is linked to the IDUA amino acid sequence, IDUA variant amino acid sequence, or a catalytically active fragment thereof, by a peptide bond or by a polypeptide linker.

19-21. (canceled)

22. The protein of claim 1, wherein the N-terminus or the C-terminus of the first Fc polypeptide is linked to the IDUA amino acid sequence, IDUA variant amino acid sequence, or a catalytically active fragment thereof.

23-27. (canceled)

28. The protein of claim 1, wherein the first Fc polypeptide contains T366S, L368A, and Y407V substitutions and the second Fc polypeptide contains a T366W substitution.

29. The protein of claim 28, wherein the first Fc polypeptide comprises an amino acid sequence having at least 95% or 100% identity to any one of SEQ ID NOS: 9-16 and 19-22; and the second Fc polypeptide comprises an amino acid sequence having at least 95% or 100% identity to any one of SEQ ID NOS: 25-32 and 35-38.

30-32. (canceled)

33. The protein of claim 1, wherein the first Fc polypeptide and/or the second Fc polypeptide includes a modification that reduces effector function, and wherein the modification that reduces effector function is the substitutions of Ala at position 234 and Ala at position 235; Ala at position 234, Ala at position 235 and Gly at position 329; or Ala at position 234, Ala at position 235 and Ser at position 329, according to EU numbering.

34. The protein of claim 33, wherein the first Fc polypeptide comprises an amino acid sequence having at least 95% or 100% identity to any one of SEQ ID NOS: 11-16, and 19-22.

35-36. (canceled)

37. The protein of claim 33, wherein the first Fc polypeptide linked to the IDUA amino acid sequence comprises an amino acid sequence having at least 95% or 100% identity to any one of SEQ ID NOS: 50-69 and 83-92.

38-45. (canceled)

46. The protein of claim 33, wherein the second Fc polypeptide comprises an amino acid sequence having at least 95% or 100% identity to any one of SEQ ID NOS: 27-32 and 35-38.

47-48. (canceled)

49. The protein of claim 1, wherein the first Fc polypeptide linked to the IDUA amino acid sequence comprises the amino acid sequence of any one of SEQ ID NOS: 50-65 and 83-92; and wherein the second Fc polypeptide comprises the amino acid sequence of any one of SEQ ID NOS: 35-38.

50. The protein of claim 49, wherein;
1) the first Fc polypeptide linked to the IDUA amino acid sequence comprises the amino acid sequence of any one of SEQ ID NOS: 50-57 and 83-86; and wherein the second Fc polypeptide comprises the amino acid sequence of any one of SEQ ID NOS: 35-36; or
2) the first Fc polypeptide linked to the IDUA amino acid sequence comprises the amino acid sequence of any one of SEQ ID NOS: 58-65 and 87-92; and wherein the second Fc polypeptide comprises the amino acid sequence of any one of SEQ ID NOS: 37-38.

51. (canceled)

52. The protein of claim 1, wherein the first Fc polypeptide linked to the IDUA amino acid sequence comprises the amino acid sequence of any one of SEQ ID NOS: 66-69; and wherein the second Fc polypeptide comprises the amino acid sequence of any one of SEQ ID NOS: 35-38.

53. The protein of claim 52, wherein:
1) the first Fc polypeptide linked to the IDUA amino acid sequence comprises the amino acid sequence of any one of SEQ ID NOS: 66-67; and wherein the second Fc polypeptide comprises the amino acid sequence of any one of SEQ ID NOS: 35-36; or
2) the first Fc polypeptide linked to the IDUA amino acid sequence comprises the amino acid sequence of any one of SEQ ID NOS: 68-69; and wherein the second Fc polypeptide comprises the amino acid sequence of any one of SEQ ID NOS: 37-38.

54-56. (canceled)

57. The protein of claim 1, wherein the protein does not include an immunoglobulin heavy and/or light chain variable region sequence or an antigen-binding portion thereof.

58. A polypeptide comprising an Fc polypeptide that is linked to an alpha-L-iduronidase (IDUA) amino acid sequence, an IDUA variant amino acid sequence, or a catalytically active fragment thereof, wherein the Fc polypeptide comprises a sequence having at least 90% identity to SEQ ID NO: 12 and contains one or more modifications that promote its heterodimerization to another Fc polypeptide.

59-64. (canceled)

65. A protein comprising the polypeptide and the other Fc polypeptide of claim 58.

66. A pharmaceutical composition comprising the protein of claim 1 and a pharmaceutically acceptable excipient.

67. A polynucleotide comprising a nucleic acid sequence encoding the polypeptide of claim 58.

68. A vector comprising the polynucleotide of claim 67.

69. A host cell comprising the polynucleotide of claim 67.

70. (canceled)

71. A method for producing a polypeptide comprising an Fc polypeptide that is linked to an IDUA amino acid sequence, IDUA variant amino acid sequence, or catalytically active fragment thereof, comprising culturing a host cell under conditions in which the polypeptide encoded by the polynucleotide of claim 67 is expressed.

72. A pair of polynucleotides comprising a first nucleic acid sequence encoding the first Fc polypeptide linked to an IDUA amino acid sequence, IDUA variant amino acid sequence, or catalytically active fragment thereof; and a second nucleic acid sequence encoding the second Fc polypeptide, as recited in claim 1.

73. One or more vectors comprising the pair of polynucleotides of claim 72.

74. A host cell comprising the pair of polynucleotides of claim 72.

75. A method for producing a protein comprising a first Fc polypeptide linked to an IDUA amino acid sequence, IDUA variant amino acid sequence, or catalytically active fragment thereof, and a second Fc polypeptide, comprising culturing a host cell under conditions in which the pair of polynucleotides of claim 72 are expressed.

76. A method of treating MPS I, the method comprising administering the protein of claim 1 to a patient in need thereof.

77-78. (canceled)

79. A method of decreasing the accumulation of a toxic metabolic product in a patient having MPS I, the method comprising administering the protein of claim 1 to the patient.

80-82. (canceled)
